(19) United States
(12) Patent Application Publication
(10) Pub. No.: US 2025/0270519 A1
(43) Pub. Date: Aug. 28, 2025

(54) CHIMAERIC PROTEINS AND THERAPEUTIC AGENTS

(71) Applicant: Oxford University Innovation Limited, Oxford (GB)

(72) Inventors: Terrence RABBITTS, London (GB); Nicolas BERY, Dierre (FR)

(21) Appl. No.: 17/924,695

(22) PCT Filed: May 12, 2021
(86) PCT No.: PCT/GB2021/051138

§ 371 Date: Nov. 11, 2022

(30) Foreign Application Priority Data
May 12, 2020 (GB) 2006974.6

(51) Int. Cl. C12N 9/10; A61K 38/00; A61P 35/00; C07K 16/40

(52) U.S. Cl. CPC C12N 9/104; A61P 35/00; C07K 16/40; A61K 38/00; C07K 2318/00; C07K 2319/00

(57) ABSTRACT

Provided is a chimaeric protein comprising a ubiquitin ligase domain and a RAS-specific endogenous targeting portion. The ubiquitin ligase domain may be a VHL E3 ligase domain, or a fragment or variant thereof having ubiquitin ligase activity; or a UBOX domain of CHIP, or a fragment or variant thereof having ubiquitin ligase activity. The RAS-specific endogenous targeting portion may be a RAS-specific DARPin; or a RAS-specific intracellular antibody. In particular, the RAS-specific endogenous targeting portion may be a KRAS-specific endogenous targeting portion (for example a KRAS-specific DARPin, or a KRAS-specific intracellular antibody). The chimaeric proteins may be used in the prevention and/or treatment of a RAS-associated disorder, such as a RAS-associated cancer or a RASopathy. Further provided is a chimaeric protein comprising a ubiquitin ligase domain and an LMO2-specific endogenous targeting portion.

### FIELD OF THE INVENTION

The present invention relates to chimaeric proteins, and to nucleic acids encoding such chimaeric proteins. The invention also relates to pharmaceutical compositions comprising the chimaeric proteins of nucleic acids of the invention. Further, the invention relates to medical uses and methods of treatment employing such pharmaceutical compositions, chimaeric proteins, or nucleic acids.

### BACKGROUND

Mutations of the KRAS oncogene represent more than 85% of all RAS family mutations and individual mutations occur at various codons giving rise to many forms of mutant KRAS protein. Recently, several macromolecules and compounds have been developed that influence the function of RAS family members. Nevertheless, only the G12C mutation of KRAS has been specifically targeted by small molecules by virtue of covalent binding of the compounds to the mutant cysteine. Several small molecules are now in clinical trials. However, only a small portion (around 12%) of mutant KRAS tumours expresses a KRAS^{G12C} protein, and are thus able to be targeted by these inhibitors. Furthermore, upon treatment with these inhibitors, a rapid adaptation has been recently described in the KRAS^{G12C} tumour population such that some cells become drug-insensitive. Therefore, new strategies are needed to specifically target the larger number of other tumours expressing mutant KRAS. Reagents that could potentially be applied to this objective are DARPins K13 and K19 that interfere specifically with KRAS. While these DARPins do not discriminate mutant from wild-type KRAS (KRAS^{WT}), they do not bind to NRAS and HRAS so that any phenotype engendered using these DARPins within cells would spare the expression, and function, of these two family members.

Previous studies involving expression of a pan-RAS-binding intracellular single domain antibody (also referred to herein as iDAb RAS) in human cell line xenografts demonstrated that tumour growth was inhibited for the duration of expressing the intracellular antibody fragment and resumed when the antibody was removed. A potential complementary direction in this context could be the addition of warheads to these macromolecules, such as E3 ligases engineered on intracellular single domains called macrodrug degraders, that have been shown to invoke proteolysis of targets. Macromolecule degraders induce the depletion of their target via the ubiquitin-proteasome system. They consist of a binder targeting a protein of interest (e.g. intracellular single domain), a linker and an E3 ligase domain. A similar protein target degradation strategy has been developed in which small molecules that bind proteins are linked to E3 ligase-binding ligands called Proteolysis Targeting Chimeras (PROTACs) or small molecule degraders.

The main advantage of the proteolysis strategy is that only a binder is required, and the binder does not need to inhibit the function of the protein. Indeed, unlike classical protein-protein inhibitors or other occupancy-driven inhibitors, the degraders rely on an event-driven mode of action and are consequently often more potent than the parental entity. Most of the current degraders target BET or kinase families and only a few target “undruggable” proteins such as transcription factors. The only PROTACs thus far applied to RAS are compounds that bind KRAS^{G12C} but these only degrade exogenous GFP-KRAS^{G12C} fusion protein and do not target endogenous KRAS^{G12C} In addition, no macromolecule or small molecule-based degrader has been shown to be specific to KRAS in the RAS family of oncogenic targets.

We report here the engineering of the KRAS-specific DARPin K19 (also referred to herein as DP KRAS) into a KRAS-specific degrader and compare the efficacy and tumour-specificity with an engineered pan-RAS degrader, made from the previously described pan-RAS intracellular single domain antibody. We show that the KRAS degrader efficiently induces endogenous KRAS degradation in vitro and in vivo and specifically inhibits mutant KRAS tumours without affecting cells with only KRAS^{WT} whereas the pan-RAS degraders inhibit all type of cells, regardless of the RAS isoform mutation. Therefore, we have exploited this KRAS-specific macrodrug to demonstrate that KRAS ablation can be an attractive way to target any mutant KRAS-expressing tumour.

It is an aim of certain aspects and embodiments of the present invention to address at least some of the shortcomings associated with the therapeutic agents known in the prior art. It is an aim of certain aspects and embodiments of the present invention to provide therapeutic agents that are suitable for use in the prevention and/or treatment of KRAS-associated cancers or RASopathies.

### SUMMARY OF THE INVENTION

In a first aspect, the invention provides a chimaeric protein comprising a ubiquitin ligase domain and an exogenous RAS-specific endogenous targeting portion.

In a second aspect, the invention provides a chimaeric protein comprising a ubiquitin ligase domain and an exogenous LMO2-specific endogenous targeting portion.

In a third aspect, the invention provides a nucleic acid molecule comprising a nucleic acid sequence encoding a chimaeric protein in accordance with the first or second aspects of the invention.

In a fourth aspect, the invention provides a pharmaceutical composition comprising a chimaeric protein of the first or second aspect of the invention and/or a nucleic acid molecule of the third aspect of the invention, and a pharmaceutically acceptable carrier.

In a fifth aspect the invention provides a method of preventing or treating a RAS-associated disorder, the method comprising providing a therapeutically effective amount of a chimaeric protein according to the first aspect of the invention to a subject in need thereof. The RAS-associated disorder may be selected from the group consisting of: a RAS-associated cancer; a RAS-associated psychiatric disorder; and a RASopathy. The chimaeric protein may be provided by administration of the protein itself, or by administration of a nucleic acid molecule encoding the protein. Either a protein or nucleic acid molecule may be provided by administration of an appropriate pharmaceutical composition of the invention.

The fifth aspect of the invention also provides the medical uses of the chimaeric proteins of the first aspect of the invention, of nucleic acid molecules of the invention encoding such chimaeric proteins, or of pharmaceutical compositions of the invention comprising such chimaeric proteins or nucleic acids. The medical use may be for preventing or treating a RAS-associated disorder. The RAS-associated disorder may be selected from the group consisting of: a RAS-associated cancer; a RAS-associated psychiatric disorder; and a RASopathy. The RAS-associated cancer may be selected from: RAS-associated lung cancer; RAS-associated pancreatic cancer; and RAS-associated colorectal cancer. Suitable examples of RASopathies and further examples of RAS-associated cancers are discussed elsewhere in the specification.

A sixth aspect of the invention provides a method of preventing or treating a condition associated with expression of LMO2, the method comprising providing a therapeutically effective amount of a chimaeric protein according to the second aspect of the invention to a subject in need thereof. The condition associated with expression of LMO2 may be selected from the group consisting of: T-cell acute lymphoblastic leukaemia (T-ALL); LMO2⁺ breast cancer; LMO2⁺ prostate cancer; and LMO2⁺ diffuse large B cell lymphoma. The chimaeric protein may be provided by administration of the protein itself, or by administration of a nucleic acid molecule encoding the protein. Either a protein or nucleic acid molecule may be provided by administration of an appropriate pharmaceutical composition of the invention.

The sixth aspect of the invention also provides the medical uses of the chimaeric proteins of the second aspect of the invention, of nucleic acid molecules of the invention encoding such chimaeric proteins, or of pharmaceutical compositions of the invention comprising such chimaeric proteins or nucleic acids. The medical use may be for preventing or treating a condition associated with expression of LMO2. The condition associated with LMO2 may be selected from the group consisting of: T-ALL; LMO2⁺ breast cancer; LMO2⁺ prostate cancer; and LMO2⁺ diffuse large B cell lymphoma. Suitably the medical use is for preventing or treating T-ALL.

The chimaeric proteins of the invention, as well as nucleic acids encoding these proteins, represent effective agents for the treatment of diseases associated with RAS or with LMO2.

Chimaeric proteins of the invention provide effective agents that achieve their biological activity by specifically inducing the transfer of their target, either RAS or LMO2, to the proteasome. Here the target protein undergoes proteolysis, and so intracellular RAS or LMO2 levels are reduced in treated cells.

Since elevated levels of RAS or LMO2, or mutant forms of RAS such as KRAS, are associated with a large number of diseases, the reduction in intracellular levels of these proteins has a beneficial therapeutic effect. This may be observed in respect of a number of different disorders associated with RAS and LMO2.

It is known that LMO2 and RAS (and particularly mutant forms of RAS) drive the progression of associated cancers (which are sometimes described as “addicted” to the proteins in question). In this case reducing the presence of the proteins prevents further cancer progression. The chimaeric proteins of the invention, and nucleic acids encoding them, represent useful agents for the treatment of RAS-associated disorders, and LMO2-associated disorders such as T-ALL.

In particular, the chimaeric proteins of the invention have been shown to be effective in the treatment of cancers associated with KRAS mutations. Specifically, the chimaeric proteins of the invention have been shown to induce regression of KRAS-associated tumours in an animal model. Without wishing to be bound by any hypothesis, the data presented in the present specification indicate that the chimaeric proteins of the invention induce apoptosis of cancerous cells expressing mutant forms of KRAS.

Indeed, the inventors have found that chimaeric proteins of the invention are able to effectively bring about tumour regression, through their induction of cancer cell apoptosis. Surprisingly they can achieve this without needing to be able to recognise specific, or indeed any, mutations in the protein (e.g. KRAS) that they target.

This is a significant advantage with respect to previous agents, many of which rely upon binding to specific mutations (such as the G12C mutation of KRAS), in order to achieve their effect. However, it is well known that there are many different RAS mutations associated with RAS-based pathologies such as cancer, and even multiple KRAS mutations responsible for different KRAS-based cancers. Since an agent that specifically targets one particular mutation will not be effective in respect of a different mutation, successful use of such agents requires the identification of the mutation present. Furthermore, not all mutations have corresponding targeting agent that are able to bind specifically to them.

The chimaeric proteins of the invention do not suffer from this disadvantage, as they can be used as “broad spectrum” agents for the treatment of RAS-associated disorders, such as RAS-associated cancers or RASopathies. This avoids the need to identify the particular mutation, or mutations, that is responsible for a patient's disease, or to develop novel targeting portions capable of binding to newly identified mutations.

Furthermore, the inventors have unexpectedly found that chimaeric proteins of the invention, are able to selectively kill cancer cells expressing mutant forms of RAS, without killing cells expressing wild-type RAS, even in embodiments where the RAS-specific endogenous targeting portion (for example the K19 DARPin) does not distinguish between mutant and wild-type forms and both wild-type and mutant forms of RAS are depleted.

As discussed in more detail elsewhere in the specification, the inventors have surprisingly found that the orientation of the respective parts of the chimaeric protein has a dramatic and unexpected impact on the ability of a chimaeric proteins of the invention to bring about the clearance of its cellular target. In an advantageous embodiment, the ubiquitin ligase domain is attached to the N-terminal region of the endogenous targeting portion. In contrast, chimaeric proteins comprising a ubiquitin ligase domain and a endogenous targeting portion wherein the endogenous targeting portion is attached to the N-terminal region of the ubiquitin ligase domain are less effective in clearing targets such as KRAS or LMO2.

As discussed further below, the ubiquitin ligase domain used in a chimaeric protein of the invention may comprise a Von Hippel-Lindau (VHL) E3 ligase as the ubiquitin ligase domain. The RAS-specific endogenous targeting portion of a chimaeric protein of the first aspect of the invention may be selected with a view to the desired target protein. For example, in the case that it is desired to reduce intracellular levels of a number of different RAS isotypes, a pan RAS-specific endogenous targeting portion of a chimaeric protein of the invention may comprise a pan-RAS intracellular antibody. When it is desired to particularly reduce intracellular levels of KRAS, a chimaeric protein of the invention may comprise a KRAS-specific DARPin as a KRAS-specific endogenous targeting portion. When it is desired to reduce intracellular levels of LMO2, an anti-LMO2 scFv may be used as an LMO2-specific endogenous targeting portion of a chimaeric protein of the second aspect of the invention.

In a suitable embodiment a chimaeric protein of the invention may comprise both a VHL ubiquitin ligase domain and one of: an anti-pan RAS intracellular antibody, an anti-KRAS DARPin, or an anti-LMO2 intracellular antibody. The inventors have found that in such embodiments it is particularly advantageous that the VHL E3 ligase domain is attached to the N-terminal region of the DARPin KRAS-specific endogenous targeting portion.

Further details of these various aspects of the invention, and of suitable embodiments, are set out below.

### BRIEF DESCRIPTION OF THE DRAWINGS

The invention will now be further described with reference in the accompanying drawings.

FIG. 1: Engineering KRAS and pan-RAS-targeted protein degradation with single domains.

(a) Effect of the fusion single domain-UBOX on RAS proteolysis assessed by Western blots. The UBOX domain was fused either on the N- or C-terminal end of the pan-RAS iDAb (iDAb RAS) or iDAb Control (Ctl), the KRAS-specific DARPin K19 (DP KRAS) or DARPin Control (DP Ctl). 24 hours after transfection of the plasmids in HCT116 cells, a Western blot was performed to determine RAS protein levels and the expression from each construct with a FLAG antibody. β-actin is the loading control. The black arrowhead shows the specific band corresponding to NRAS protein in the observed doublet. (b) The histograms display the quantification of each RAS isoform from two independent experiments (grey dots) normalised to untransfected cells. (c) Effect of the fusion single domain-VHL on RAS proteolysis assessed by Western blots as in (a). (d) Quantification of two independent experiments (grey dots) normalised to the untransfected cells as in (b). In (a, b) and (c, d), the best pan-RAS and KRAS degraders have been highlighted in orange and blue respectively. (e) HCT116 transfected cells with the indicated constructs were treated with DMSO (−) or 0.8 μM of the proteasome inhibitor epoxomicin (+) for 18 hours. KRAS degradation was evaluated by western blots. (f) KRAS degradation quantification of two independent experiment normalised to the untransfected cells. Each experiment was performed in duplicate. Error bars in b, d and f are mean±SEM from two biological repeats. (g) Scheme recapitulating the effect of the two RAS degraders: the pan-RAS degrader (iDAb RAS-UBOX) degrades all RAS isoforms through the proteasome machinery while the KRAS degrader (VHL-DP KRAS) only induces the degradation of KRAS without affecting NRAS or HRAS.

FIG. 2: Characterisation of pan-RAS and KRAS degraders in H358 cancer cells.

Western blot of RAS and effectors protein levels following induction of the macrodrug degraders for dose response and time course analysis. (a, b) Dose response experiment with the indicated doxycycline (dox) concentrations incubated for 18 hours in H358 cells expressing either iDAb Ctl-UBOX or iDAb RAS-UBOX (a), VHL-DP Ctl or VHL-DP KRAS (b). KRAS, NRAS and HRAS protein levels were assessed by western blot. FLAG antibody shows the expression of the constructs and α-tubulin is the loading control. The black arrowhead shows the specific band corresponding to NRAS protein in the observed doublet. (c, d) Time response experiment of H358 cells expressing the same degraders as in (a, b). Effect of the pan-RAS (c) and KRAS degraders (d) on KRAS, NRAS and HRAS protein levels was determined using β-actin as the loading control. (e, f) Effect of the pan-RAS (e) and KRAS degraders (f) on RAS dependent pathways was assessed and the β-actin loading control is the same as in panels (c, d). Each experiment was performed twice.

FIG. 3: Efficacy of pan-RAS and KRAS degraders in cancer cells.

Western blot of RAS family protein levels following induction of the macrodrug degraders. (a, b) Efficacy of the pan-RAS (a) and KRAS degraders (b) to deplete their target(s) in various human cell lines with the indicated variety of RAS mutations. KRAS, NRAS and HRAS protein levels were judged by Western blots after 72 hours of doxycycline treatment of the cells at 0.2 μg·mL⁻¹ (+) or not induced (−). The lines used were mutant KRAS cell lines H358, MIA PaCa2 and A549, mutant NRAS H1299, HT1080 and mutant HRAS T24 cell lines and RAS^{WT} HCC827 and MRC5 cells. Expression of the degraders is shown with the FLAG antibody. β-actin is the loading control. Each experiment in (a-b) was performed twice.

FIG. 4: The pan-RAS degrader inhibits RAS signalling pathways in all cell lines.

Effect of the pan-RAS degrader on RAS downstream signalling pathways of various cell lines was examined by Western blot analysis: (a) H358 (KRAS^{G12C}), (b) MIA PaCa-2 (KRAS^{G12C}) (c) A549 (KRAS^{G12S}), (d) H1299 (NRAS^{Q61K}), (e) HT1080 (NRAS^{Q61K}), (f) T24 (HRAS^{G12V}) (g) HCC827 (RAS^{WT}) and (h) MRC5 (RAS^{WT}). All the cell lines stably express dox inducible iDAb RAS-UBOX or its negative control iDAb Ctl-UBOX. FLAG antibody is used to determine iDAbs expression when induced with 0.2 μg·mL⁻¹ of doxycycline for 72 hours (+) or not induced (−). β-actin is the loading control. Each experiment was performed at least three times.

FIG. 5: KRAS degrader only inhibits RAS signalling pathways in cancer cell lines expressing mutant KRAS.

The effect of KRAS degrader on RAS downstream signalling pathways of various cell lines was examined by Western blot analysis: (a) H358 (KRAS^{G12C}), (b) MIA PaCa-2 (KRAS^{G12C}) (c) A549 (KRAS^{G12S}), (d) H1299 (NRAS^{Q61K}), (e) HT1080 (NRAS^{Q61K}), (f) T24 (HRAS^{G12V}) (g) HCC827 (RAS^{WT}) and (h) MRC5 (RAS^{WT}). All the cell lines stably express dox inducible VHL-DP KRAS or its negative control VHL-DP Ctl. FLAG antibody is used to determine DARPins (DP) expression when induced with 0.2 μg·mL⁻¹ of doxycycline for 72 hours (+) or not induced (−). β-actin is the loading control. (i) Quantifications of pAKT^{S473}/AKT and pERK/ERK signals from FIGS. 4 & 5. The signals were normalised to the no dox (−) condition. Each experiment in (a-h) was performed at least three times. Error bars in (i) are mean±SEM from at least three biological repeats.

FIG. 6: The KRAS degrader specifically inhibits the proliferation of cells expressing mutant KRAS. Assessment of the effect of pan-RAS and KRAS degraders on 2D-adherent and 3D spheroids proliferation of various cell lines. Mutant KRAS lines (a) H358, (b) MIA PaCa2 and (c) A549 stable cell lines. (d-e) Mutant NRAS lines: (d) H1299 and (e) HT1080. (f) Mutant HRAS T24 cell line. (g-h) Wild type RAS cell lines: (g) HCC827 and (h) MRC5. Note that MRC5 cells do not grow as spheroids in 3D low attachment plates, since as untransformed cells, they need anchorage to grow. All proliferation assays (2D and 3D) were normalised to the no dox condition for each cell line. The dotted lines represent the dox-treated cells while the plain lines show the no dox conditions. Each experiment in (a-h) was performed at least three times. Error bars in (a-h) are mean±SD from at least three biological repeats.

FIG. 7: KRAS protein depletion by the KRAS degrader leads to apoptosis of mutant KRAS dependent cells. (a) Western blot analysis of the apoptosis indicators, viz. cleaved PARP (cPARP) and cleaved caspase 3 (cCASP3) induced after expression of the degraders in H358 cells in a time response experiment. (b, c) Western blot analysis of the two apoptosis markers after expression of the degraders in 2D-adherent cultures of all 8 cell lines tested in this study after 72 hours of doxycycline treatment at 0.2 μg·mL⁻¹ (+) or not induced (−). α-tubulin is the loading control. The two arrows indicate the cleaved caspase 3 fragments at 17/19 kDa. Each experiment in (a-c) was performed twice.

FIG. 8: The KRAS degrader induces regression of mutant KRAS H358 tumours. 4.5×10⁶ H358 cells expressing either FLuc/iDAb RAS-UBOX or FLuc/VHL-DP KRAS were injected subcutaneously into CD-1 nude mice. After tumours reached 3-4 mm diameter, animals were separated into groups of 5 mice and treated or not with doxycycline (dox) in drinking water and food. (a) Tumour volumes were measured using digital calipers and normalised to 1 at the starting day of dox treatment (day 1) and monitored for 20 days (four to five mice per group, mean±SD). (b) Waterfall plot representing the percentage of change in tumour volume of individual tumours after 20 days of −/+ dox treatment. The percentage of change in tumour volume was calculated as follow: V_{final}−Vᵢₙᵢₜᵢₐₗ/Vᵢₙᵢₜᵢₐₗ×100. (c, d) Tumour burden from H358-FLuc/iDAb RAS-UBOX (c) and H358-FLuc/VHL-DP KRAS (d) was assessed by bioluminescence imaging at the end of the experiment (day 20). Photon flux (i.e. luminescence signal) was quantified for each group at day 20 (mean±SEM). (e, f) Western blot analysis of H358 tumour lysates from H358-FLuc/iDAb RAS-UBOX tumours (e) or from H358-FLuc/VHL-DP KRAS tumours (f) after 48 hours of dox treatment compared to non-treated H358 mice tumours taken at the end point of the experiment (20 days). The black arrowhead indicates the specific band corresponding to FLAG-tagged VHL-DP KRAS protein.

FIG. 9: Characterisation of DARPin control and the degraders. (a) BRET donor saturation assay between DARPin KRAS (DP KRAS) or DARPin Control (DP Ctl) as acceptor and KRAS^{WT}, KRAS^{G12D}, NRAS^{Q61H} or HRAS^{G12V} as donors. (b) Co-immunoprecipitation of 3×FLAG-KRAS^{WT} and 3×FLAG-KRAS^{G12D} with the DARPin-GFP² fusions in 10% foetal bovine serum. IP: Immunoprecipitation, WCE: whole-cell extract. DARPin E3.5 is a non-relevant DARPin. (c) BRET competition by the indicated DARPins of interaction between KRAS^{G12D} and CRAF^{FL} or (d) KRAS^{G12D} dimerization. (−) means no competitor was used. Each experiment was performed twice (a, b) or four times (c, d). Error bars are mean±SD of biological repeats (a, c, d). (e-h) DNA and protein sequences of iDAb RAS-UBOX (pan-RAS degrader), iDAb Ctl-UBOX, VHL-DP KRAS (KRAS degrader) and VHL-DP Ctl.

FIG. 10: RAS degraders induce their endogenous target degradation through the proteasome machinery. (a) H358 cells expressing iDAb Ctl-UBOX, iDAb RAS-UBOX, VHL-DP Ctl or VHL-DP KRAS were either untreated (−), treated with dox only (0.5 μg·mL⁻¹) or treated with dox and epoxomicin (0.8 M) for 18 hours. RAS protein level was determined by Western blot using a pan-RAS antibody. α-tubulin is the loading control. (b) Quantitative real-time PCR was performed after 24 hours of treatment with 0.2 μg·mL⁻¹ of dox (+) or untreated (−) in H358 stable cell lines. No dox conditions were standardised to a value of 1.0 and DUSP6 mRNA abundancy is represented as a fold-change relative to that value. Data are based on biological duplicates and are normalised to GAPDH. Error bars denote SEM.

FIG. 11: Effect of the parental iDAb macrodrug on RAS signalling pathways of various cell lines. Effect of iDAb RAS parental single domain (non-degrader) on RAS signalling pathways of various cell lines: (a) H358 (KRAS^{G12C}), (b) MIA PaCa-2 (KRAS^{G12C}), (c) A549 (KRAS^{G12S}), (d) H1299 (NRAS^{Q61K}), (e) HT1080 (NRAS^{Q61K}), (f) T24 (HRAS^{G12V}), (g) HCC827 (RAS^{WT}) and (h) MRC5 (RAS^{WT}). All the cells stably express dox-inducible iDAb RAS-GFP² and its negative control iDAb Ctl-GFP². FLAG antibody is used to show iDAb expression when induced with 0.2 μg·mL⁻¹ of doxycycline for 72 hours (+) or not induced (−). β-actin is the loading control. Each experiment in (a-h) was performed at least three times.

FIG. 12: Effect of the parental DARPin macrodrug on RAS signalling pathways of various cell lines. The effect of DP KRAS parental single domain (non-degrader) on RAS signalling pathways of various cell lines: (a) H358 (KRAS^{G12C}), (b) MIA PaCa-2 (KRAS^{G12C}) (c) A549 (KRAS^{G12S}), (d) H1299 (NRAS^{Q61K}), (e) HT1080 (NRAS^{Q61K}), (f) T24 (HRAS^{G12V}) (g) HCC827 (RAS^{WT}) and (h) MRC5 (RAS^{WT}). All the cells stably express dox-inducible DP KRAS-GFP² and its negative control DP Ctl-GFP². FLAG antibody is used to show DARPins (DP) expression when induced with 0.2 μg·mL⁻¹ of doxycycline for 72 hours (+) or not induced (−). β-actin is the loading control. (i) Comparative quantifications of pAKT^{S473}/AKT and pERK/ERK signals affected by iDAb RAS and DP KRAS fused to GFP² from Figures S3 & S4. The signals were normalised to the no dox (−) condition. Each experiment in (a-h) was performed at least three times. Error bars in (i) are mean±SEM from at least three biological repeats.

FIG. 13: Effect of the parental iDAb and DARPin macrodrugs on 2D-adherent proliferation assays of various cell lines.

Cells were grown as adherent cultures and macrodrugs induced by doxycycline treatment for assessment of the effect of iDAbs and DPs fused to GFP² on proliferation of the mutant KRAS cell lines: (a) H358, (b) MIA PaCa2 and (c) A549. (d-e) Effect of the single domains on 2D-adherent proliferation of NRAS mutant cell lines: (d) H1299 and (e) HT1080. (f) Effect of iDAb-GFP² and DP-GFP² fusions on 2D-adherent proliferation of mutant HRAS T24 cell lines. (g-h) Effect of the parental single domains on 2D-adherent proliferation of the RAS' cell lines: (g) HCC827 and (h) MRC5. All proliferation assays were normalised to the no dox condition for each cell line. The plain lines represent the dox-treated cells while the dotted lines show the no dox conditions. Each experiment in (a-h) was performed at least three times. Error bars in (a-h) are mean±SD from at least three biological repeats.

FIG. 14: Characterisation of H358-FLuc and H1299-FLuc clones and effect of the degraders on H1299 tumours xenografts. (a) Western blot analysis of the RAS downstream signalling pathways RAS/RAF/MEK/ERK and PI3K/AKT in H1299-FLuc and H358-FLuc cloned cells. FLAG antibody is used to show expression of iDAbs and DARPins when induced with 0.2 μg·mL⁻¹ of doxycycline for 72 hours ((+) or not induced (−)). KRAS, NRAS and HRAS protein levels were also assessed by Western blot to confirm the proteolysis of the degraders target(s). β-actin is the loading control. The black arrowheads indicate the specific band corresponding to pAKT^{S413} and NRAS proteins. (b, c) Cell growth assay of each clone for H358 (b) and H1299 (c). Cells were grown with or without 0.2 μg·mL⁻¹ of dox and counted every two days for 6 days to determine their growth. Each experiment (b, c) was performed twice. Error bars in (b, c) are mean±SD from two biological repeats. (d, e) 5×10⁶ H1299 cells inducibly expressing either FLuc/iDAb RAS-UBOX (d) or FLuc/VHL-DP KRAS (e) were injected subcutaneously into CD-1 nude mice. After tumours reached 2-3 mm diameter, animals were separated into groups of 3-5 mice and treated or not with doxycycline (+/− dox) in drink and food. Tumour burden was assessed by bioluminescence imaging at the end of the experiment (day 20). Photon flux (i.e. luminescence signal) was quantified for each group at day 20 (mean±SEM). *P<0.05 and ns: non-significant. (f, g) Western blot analysis of H1299 tumour lysates after 20 days of −/+ doxycycline treatment in H1299-FLuc/iDAb RAS-UBOX (f) and H1299-FLuc/VHL-DP KRAS (g). The black arrowhead indicates the specific band corresponding to FLAG-tagged VHL-DP KRAS protein.

FIG. 15: Chimaeric proteins in accordance with the second aspect of the invention are able to reduce intracellular LMO2 in vitro. FIG. 15 shows the results of studies in which a HEK293 cell line expression LMO2 was transfected with one of a number of test plasmids incorporating VH576, an intracellular single domain antibody to LMO2. VH576 was fused with a VHL E3 ligase domain to produce a chimaeric protein in accordance with the second aspect of the invention (referred to as “iDAb LMO2-VHL” in the Figure, and VH576-VHL elsewhere in the specification), and with Green Fluorescent Protein (GFP) or Cerebron E3 ligase (CRBN) to produce suitable control chimaeric proteins. An anti-RAS VHY6 fusion protein with CRBN was used as a negative control for the endogenous targeting portion. After 24 hours, protein extracts were prepared, separated by SDS-PAGE and Western blotted using an anti-LMO2 antibody and β-actin as loading control. Panel A: shows the results of Western blot analysis; Panel B: shows quantification of LMO2 in lanes from cells treated with the chimaeric proteins of the invention or controls (as compared to untransfected signal); and Panel C: shows expanded quantification of LMO2 in cells treated with VHL-iDAb LMO2 (VHL-VH576 a chimaeric protein in accordance with the second aspect of the invention) and iDAb LMO2-VHL (VH576-VHL), demonstrating the importance of the orientation of the ubiquitin ligase domain and LMO2-specific endogenous targeting portion in embodiments of this second aspect of the invention.

### DETAILED DESCRIPTION OF THE INVENTION

The invention will now be further explained with reference to the following paragraphs, and the definitions provided therein.

### “Chimaeric Proteins” of the Invention

The invention relates to chimaeric proteins comprising a ubiquitin ligase domain and either a RAS-specific endogenous targeting portion (chimaeric proteins of the first aspect of the invention) or an LMO2-specific endogenous targeting portion (chimaeric proteins of the second aspect of the invention). Chimaeric proteins are made up of sequences from at least two proteins. These may be artificial proteins (such as DARPins or intracellular antibodies), o naturally occurring proteins, such as ubiquitin ligases. It will be appreciated that the chimaeric proteins are not naturally occurring themselves. Suitably the ubiquitin ligase domain and the RAS-specific or LMO2-specific endogenous targeting portion are each derived from separate proteins.

The proteins from which the requisite domains or portions are derived may be referred to as “parent” proteins in the context of the present disclosure. Parent proteins may be used for the generation of fragments or variants that can be used in the chimaeric proteins of the invention.

In order to be suitable for incorporation in a chimaeric protein of the invention a fragment or variant of a parent protein should retain some or all of the biological activity of the parent protein (for example ubiquitin ligase activity or the ability to specifically bind to LMO2 or a RAS, such as KRAS).

A fragment derived from a parent protein will share 100% identity with a corresponding portion of the parent protein, though it will not comprise 100% of the full-length parent protein's sequence.

Suitably, a fragment of a parent protein may comprise at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% of the full-length sequence of the parent protein. By way of example, a variant of a parent protein that may be incorporated in a chimaeric protein of the invention may share at least at least 91%, at least 92%, at least 93%, at least 94%, at least 95%, at least 96%, at least 97%, at least 98%, or at least 99% of the full-length sequence of the parent protein.

Considered in another way, a fragment may comprise 10 or more contiguous amino acid residues, for example 20 or more, 30 or more, or 40 or more contiguous amino acid residues from the parent protein sequence.

In contrast with such fragments, a variant of a parent protein incorporates one or more changes as compared to the sequence of the parent protein from which it is derived. Suitably a variant of a parent protein that may be incorporated in a chimaeric protein of the invention may share at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% identity with a corresponding portion of the parent protein. By way of example, a variant of a parent protein that may be incorporated in a chimaeric protein of the invention may share at least at least 91%, at least 92%, at least 93%, at least 94%, at least 95%, at least 96%, at least 97%, at least 98%, or at least 99% identity with a corresponding portion of the parent protein.

Suitably, a variant of a parent protein that may be incorporated in a chimaeric protein of the invention may share up to 99%, up to 98%, up to 97%, up to 96%, up to 95%, up to 94%, up to 93%, up to 92%, up to 91%, or up to 90% identity with a corresponding portion of the parent protein.

A variant in the present context comprises at least one modification as compared to the parent protein sequence. “Modification” as used herein refers to any change made to an amino acid sequence such that its sequence is not the same as that of the parent protein. Suitably a variant of a parent protein may comprise at least 2, 3, 4, 5, 10, or 15 amino acid modifications as compared to the parent protein sequence. Suitable modifications may include substitution or deletion of the amino acid residue present in the parent protein, or addition of amino acid residues not present in the parent protein sequence.

### “Endogenous Targeting Portions”

A chimaeric protein of the invention comprises an endogenous targeting portion in combination with a ubiquitin ligase domain. The endogenous targeting portion, whether specific for RAS, or a particular RAS isoform, or specific for LMO2, is a polypeptide component of a chimaeric protein of the invention that confers on the chimaeric protein the ability to bind specifically to its corresponding target.

An endogenous targeting portion suitable for use in a chimaeric protein of the invention may be any endogenous polypeptide sequence capable of binding specifically to the chosen target. The skilled person will be aware of many suitable assays by which the ability to bind specifically to targets (such as pan RAS, KRAS, HRAS, NRAS, or LMO2) may be assessed.

Endogenous targeting portions, for the purpose of the present invention, should be understood to be targeting portions that do not naturally arise in the intracellular environment. For example, an endogenous RAS targeting portion suitable for use in the chimaeric proteins of the invention would not encompass a naturally occurring intracellular RAS-binding protein, or a fragment of such a naturally occurring intracellular RAS-binding protein.

By way of example, suitable endogenous targeting portions may be selected from the group consisting of: DARPins (or fragments or variants of DARPins); and intracellular antibodies (or fragments or variants of intracellular antibodies).

DARPins are suitable for use as endogenous targeting portions in accordance with the present disclosure as they are not naturally occurring molecules. Thus, in a suitable embodiment, the endogenous targeting portion is a DARPin. Affinity maturation used during the production of DARPins means that these agents are able to achieve levels of affinity for their targets that are much higher affinity than naturally occurring agents.

Intracellular antibodies are also suitable for use as endogenous targeting portions in accordance with the present disclosure as they are also not naturally occurring proteins (antibodies having to be modified in order to be suitable for intracellular use). Thus, in a suitable embodiment, the endogenous targeting portion is an intracellular antibody.

The ability of an endogenous targeting portion, such as a DARPin or intracellular antibody, to bind to its target may be determined by an appropriate binding assay. In the case of a pan RAS-specific endogenous targeting portion this may be an appropriate pan-RAS binding assay. In the case of a KRAS-specific endogenous targeting portion this may be an appropriate KRAS binding assay. In the case of an HRAS-specific endogenous targeting portion this may be an appropriate HRAS binding assay. In the case of an NRAS-specific endogenous targeting portion this may be an appropriate NRAS binding assay. In the case of an LMO2-specific endogenous targeting portion this may be an appropriate LMO2 binding assay.

Endogenous targeting portions, and particularly antibody or DARPin endogenous targeting portions, may serve as parent proteins from which fragments or variants suitable for use in embodiments of the invention, may be generated, in keeping with the considerations set out elsewhere in the specification.

The ability of fragments or variants of endogenous targeting portions, such as DARPins or intracellular antibodies, to bind to their target may be determined by an appropriate binding assay. In the case of a fragment or variant of a pan RAS-specific DARPin or intracellular antibody this may be an appropriate pan-RAS binding assay. In the case of a fragment or variant of a KRAS-specific DARPin or intracellular antibody this may be an appropriate KRAS binding assay. In the case of a fragment or variant of an HRAS-specific DARPin or intracellular antibody this may be an appropriate HRAS binding assay. In the case of a fragment or variant of an NRAS-specific DARPin or intracellular antibody this may be an appropriate NRAS binding assay. In the case of a fragment or variant of an LMO2-specific DARPin or intracellular antibody this may be an appropriate LMO2 binding assay.

In the context of the present disclosure, references to “an endogenous targeting portion” that do not specify the target should be taken, except for where the context requires otherwise, as being applicable to all chimaeric proteins of the invention (including chimaeric proteins in accordance with the first aspect of the invention, and also chimaeric proteins in accordance with the second aspect of the invention).

### RAS

The RAS family of proteins comprises three isoforms: KRAS, HRAS and NRAS. The various isoforms share common structural elements, and each acts as an intracellular signalling agent.

### Pan RAS

For the purposes of the present specification, the term “pan RAS” should be taken as encompassing any RAS family isoform. This includes, but is not limited to, KRAS, HRAS and NRAS.

### KRAS

KRAS is an intracellular protein, and part of the RAS/MAPK pathway. The amino acid sequence of human wild-type KRAS is set out in SEQ ID NO: 1.

For the purposes of the present disclosure, references to “wild-type” KRAS should be taken as referring to a form of KRAS that does not comprise any mutations as compared to the amino acid sequence of SEQ ID NO: 1.

“Mutant” forms of KRAS will comprise at least one modification as compared to the sequence of SEQ ID NO: 1. Mutant forms of KRAS are responsible for KRAS-associated cancers including, but not limited to: lung adenocarcinoma; mucinous carcinoma; ductal carcinoma of the pancreas; and colorectal cancer. Specific mutations may be referred to with reference to the particular modifications that they incorporate, such as KRAS^{G12C}.

Unless the context requires otherwise, references to “KRAS” in the present document will be taken as encompassing both wild-type and mutant forms of KRAS.

Cellular levels of KRAS may be reduced by use of chimaeric proteins of the first aspect of the invention that comprise either a pan-RAS specific endogenous targeting portion, or a KRAS-specific endogenous targeting portion.

HRAS

The intracellular protein HRAS is also known as “transforming protein 21”. The amino acid sequence of human wild-type HRAS is set out in SEQ ID NO: 2.

For the purposes of the present disclosure, references to “wild-type” HRAS should be taken as referring to a form of HRAS that does not comprise any mutations as compared to the amino acid sequence of SEQ ID NO: 2.

“Mutant” forms of HRAS will comprise at least one modification as compared to the sequence of SEQ ID NO: 2. Mutant forms of HRAS are responsible for HRAS-associated cancers, including, but not limited to: bladder cancer; thyroid cancer; salivary duct carcinoma; epithelial-myoepithelial carcinoma; and kidney cancers. Specific mutations may be referred to with reference to the particular modifications that they incorporate.

Unless the context requires otherwise, references to “HRAS” in the present document will be taken as encompassing both wild-type and mutant forms of HRAS.

Cellular levels of HRAS may be reduced by use of chimaeric proteins of the first aspect of the invention that comprise either a pan-RAS specific endogenous targeting portion, or an HRAS-specific endogenous targeting portion.

### NRAS

NRAS is so called because of its identification in the context of neuroblastoma cells. The amino acid sequence of human wild-type NRAS is set out in SEQ ID NO: 3.

For the purposes of the present disclosure, references to “wild-type” NRAS should be taken as referring to a form of NRAS that does not comprise any mutations as compared to the amino acid sequence of SEQ ID NO: 3.

“Mutant” forms of HRAS will comprise at least one modification as compared to the sequence of SEQ ID NO: 3. Mutant forms of NRAS are responsible for NRAS-associated cancers, including, but not limited to: melanoma. Specific mutations may be referred to with reference to the particular modifications that they incorporate.

Unless the context requires otherwise, references to “NRAS” in the present document will be taken as encompassing both wild-type and mutant forms of NRAS.

Cellular levels of NRAS may be reduced by use of chimaeric proteins of the first aspect of the invention that comprise either a pan-RAS specific endogenous targeting portion, or an NRAS-specific endogenous targeting portion.

### “A RAS-Specific Endogenous Targeting Portion”

A RAS-specific endogenous targeting portion is a polypeptide component of a chimaeric protein of the invention that confers on the chimaeric protein the ability to bind specifically to RAS.

A RAS-specific endogenous targeting portion suitable for use in a chimaeric protein of the invention may be any endogenous polypeptide sequence capable of binding specifically to RAS. The skilled person will be aware of many suitable assays by which the ability to bind specifically to RAS may be assessed.

In a suitable embodiment, the RAS-specific endogenous targeting portion is selected from the group consisting of: a RAS-specific DARPin; and a RAS-specific intracellular antibody.

### “A Pan RAS-Specific Endogenous Targeting Portion”

A pan RAS-specific endogenous targeting portion is a polypeptide component of a chimaeric protein of the invention that confers on the chimaeric protein specific pan RAS binding ability.

In the context of the present disclosure, this should be taken as meaning the ability to specifically bind a plurality of RAS isoforms (such as those selected from the group consisting of KRAS, HRAS and NRAS), without significant binding to other non-RAS intracellular proteins. Suitably a pan RAS-specific endogenous targeting portion may be able to bind to all three of the RAS isoforms KRAS, HRAS and NRAS.

A pan RAS-specific endogenous targeting portion suitable for use in a chimaeric protein of the invention may be any endogenous polypeptide sequence capable of specific pan RAS binding.

The skilled person will be aware of many suitable assays by which the ability to bind specifically to a plurality of RAS isoforms may be assessed.

In a suitable embodiment, the pan RAS-specific endogenous targeting portion is selected from the group consisting of: a pan RAS-specific intracellular antibody; and a pan RAS-specific DARPin. Such pan RAS-specific intracellular antibodies or DARPins may constitute suitable parent proteins for the generation of pan RAS specific fragments or variants of such antibodies or DARPins.

An example of a pan RAS intracellular antibody suitable for use as a pan RAS-specific endogenous targeting portion in accordance with the present invention is set out in SEQ ID NO: 4. This intracellular antibody is referred to as “iDAb” (intracellular antibody single domain fragment) on occasion within this specification.

The pan RAS intracellular antibody of SEQ ID NO: 4 represents a suitable parent protein from which fragments or variants may be generated, as discussed elsewhere in the specification. In a suitable embodiment, a pan RAS-specific endogenous targeting portion comprises the amino acid sequence set out in SEQ ID NO: 4; a pan RAS-binding variant of SEQ ID NO:4; or a pan RAS-binding fragment of SEQ ID NO: 4 or its variants.

A pan RAS-specific endogenous targeting portion suitable for use in a chimaeric protein of the invention may share at least 85% identity with the amino acid sequence of SEQ ID NO: 4.

In a suitable embodiment, the pan RAS-specific endogenous targeting portion comprises the amino acid sequence set out in SEQ ID NO: 4; or a pan RAS-binding fragment thereof.

In a suitable embodiment, the pan RAS-specific endogenous targeting portion consists of the amino acid sequence set out in SEQ ID NO: 4.

As discussed elsewhere in the specification, chimaeric proteins of the invention comprising the pan RAS-specific endogenous targeting portion of SEQ ID NO: 4 may comprise this sequence (or its fragments or variants) in combination with a ubiquitin ligase domain comprising the UBOX domain of CHIP.

The exemplary chimaeric protein of the invention “UBOX-iDAb” (SEQ ID NO: 5) represents an example of a chimaeric protein comprising pan RAS-specific endogenous targeting portion based upon SEQ ID NO: 4 and a ubiquitin ligase domain comprising the UBOX domain of CHIP.

### “A KRAS-Specific Endogenous Targeting Portion”

A KRAS-specific endogenous targeting portion is a polypeptide component of a chimaeric protein of the invention that confers on the chimaeric protein the ability to bind specifically to KRAS.

A KRAS-specific endogenous targeting portion suitable for use in a chimaeric protein of the invention may be any endogenous polypeptide sequence capable of binding specifically to KRAS. The skilled person will be aware of many suitable assays by which the ability to bind specifically to KRAS may be assessed.

In a suitable embodiment, the KRAS-specific endogenous targeting portion is selected from the group consisting of: a KRAS-specific DARPin; and a KRAS-specific intracellular antibody.

In a suitable embodiment, the KRAS-specific endogenous targeting portion is a DARPin. Affinity maturation used during the production of DARPins means that these agents are able to achieve very high affinity for their targets, in this case KRAS, and much higher affinity than naturally occurring agents.

An exemplary DARPin that may be used as a KRAS-specific endogenous targeting portion in a chimaeric protein of the invention is designated K19 by the inventors. The amino acid sequence of K19 is set out in SEQ ID NO: 6, and a DNA sequence encoding K19 is set out in SEQ ID NO: 7.

An alternative DARPin that may be used as a KRAS-specific endogenous targeting portion in a chimaeric protein of the invention is designated K13 by the inventors. The amino acid sequence of K13 is set out in SEQ ID NO: 8, and a DNA sequence encoding K13 is set out in SEQ ID NO: 9.

The data set out in the Examples illustrate that chimaeric proteins of the invention comprising K19 and chimaeric proteins of the invention comprising K13 are both able to bring about an effective reduction in levels of cellular KRAS. Chimaeric proteins of the invention comprising K13 as a KRAS-specific endogenous targeting portion induce KRAS degradation but to a lesser extent than those in which K19 is used as a KRAS-specific endogenous targeting portion. The inventors believe that this difference is possibly due to K13 having a lower affinity toward KRAS than does K19 (˜30 nM versus 10 nM respectively).

The DARPins K19 and K13 represent suitable parent proteins from which fragments or variants may be generated, as discussed elsewhere in the specification. In a suitable embodiment, the KRAS-specific endogenous targeting portion comprises an amino acid sequence selected from the group consisting of: SEQ ID NO: 6; a KRAS-binding variant of SEQ ID NO:6; a KRAS-binding fragment of SEQ ID NO: 6 or its variants; SEQ ID NO: 8; a KRAS-binding variant of SEQ ID NO:8; and a KRAS-binding fragment of SEQ ID NO: 8 or its variants.

A KRAS-specific endogenous targeting portion suitable for use in a chimaeric protein of the invention may share at least 85% identity with the amino acid sequence of SEQ ID NO: 6.

In a suitable embodiment, the KRAS-specific endogenous targeting portion comprises the amino acid sequence set out in SEQ ID NO: 6; or a KRAS-binding fragment thereof.

In a suitable embodiment, the KRAS-specific endogenous targeting portion consists of the amino acid sequence set out in SEQ ID NO: 6.

A fragment or variant of the sequence set out in SEQ ID NO: 6 may retain a sufficient number of the KRAS-binding tryptophan repeats present in SEQ ID NO: 6 to retain effective specific binding of KRAS. For the avoidance of doubt, the KRAS-binding tryptophan repeats in SEQ ID NO: 6 are residues 35, 37, 45 and 46 of this sequence. Suitably a fragment or variant of the KRAS-specific endogenous targeting portion set out in SEQ ID NO: 6 may comprise at least 3 of the KRAS-binding tryptophan repeats of SEQ ID NO: 6. Suitably a fragment or variant of the KRAS-specific endogenous targeting portion set out in SEQ ID NO: 6 may comprise all 4 of the KRAS-binding tryptophan repeats of SEQ ID NO: 4.

A KRAS-specific endogenous targeting portion suitable for use in a chimaeric protein of the invention may share at least 85% identity with the amino acid sequence of SEQ ID NO: 8.

In a suitable embodiment, the KRAS-specific endogenous targeting portion comprises the amino acid sequence set out in SEQ ID NO: 8; or a KRAS-binding fragment thereof.

In a suitable embodiment, the KRAS-specific endogenous targeting portion consists of the amino acid sequence set out in SEQ ID NO: 8.

In a suitable embodiment, the KRAS-specific endogenous targeting portion is a KRAS-specific intracellular antibody.

The amino acid sequences of suitable KRAS-specific intracellular antibodies that may be used as KRAS-specific endogenous targeting portions in the chimaeric proteins of the invention are set out in SEQ ID NOs: 10 and 11. These novel KRAS-specific intracellular antibodies have respectively been named P2-E2 and P2-F3 by the inventors.

In a suitable embodiment, a KRAS-specific endogenous targeting portion of a chimaeric protein of the invention comprises the amino acid sequence set out in SEQ ID NO: 10, or a KRAS-binding fragment of the amino acid sequence set out in SEQ ID NO: 10, or a KRAS-binding variant of the amino acid sequence set out in SEQ ID NO: 10 or a fragment thereof.

A KRAS-specific endogenous targeting portion suitable for use in a chimaeric protein of the invention may share at least 85% identity with the amino acid sequence of SEQ ID NO: 10.

In a suitable embodiment, the KRAS-specific endogenous targeting portion comprises the amino acid sequence set out in SEQ ID NO: 10; or a KRAS-binding fragment thereof.

In a suitable embodiment, the KRAS-specific endogenous targeting portion consists of the amino acid sequence set out in SEQ ID NO: 10.

In a suitable embodiment, a KRAS-specific endogenous targeting portion of a chimaeric protein of the invention comprises the amino acid sequence set out in SEQ ID NO: 11, or a KRAS-binding fragment of the amino acid sequence set out in SEQ ID NO: 11, or a KRAS-binding variant of the amino acid sequence set out in SEQ ID NO: 11 or a fragment thereof.

A KRAS-specific endogenous targeting portion suitable for use in a chimaeric protein of the invention may share at least 85% identity with the amino acid sequence of SEQ ID NO: 11.

In a suitable embodiment, the KRAS-specific endogenous targeting portion comprises the amino acid sequence set out in SEQ ID NO: 11; or a KRAS-binding fragment thereof.

In a suitable embodiment, the KRAS-specific endogenous targeting portion consists of the amino acid sequence set out in SEQ ID NO: 11.

So useful are the scFvs of SEQ ID NO: 10 and SEQ ID NO: 11 that they give rise to further aspects of the invention. Thus, in a seventh aspect, the invention provides a KRAS-specific binding agent comprising SEQ ID NO: 10 or an antigen-binding fragment or variant thereof. In an eighth aspect, the invention provides a KRAS-specific binding agent comprising SEQ ID NO: 11 or an antigen-binding fragment or variant thereof.

The ability of fragments or variants of the scFvs of SEQ ID NO: 10 or SEQ ID NO: 11 to bind to antigens may be determined by an appropriate KRAS binding assay, as discussed elsewhere in the present specification.

An antigen-binding fragment of SEQ ID NO: 10 may comprise up to 300 contiguous amino acid residues of the parent protein. For example, a suitable antigen-binding fragment of SEQ ID NO: 10 may comprise up to 299, up to 298, up to 297, up to 296, up to 295, up to 294, up to 293, up to 292, up to 291, or up to 290 contiguous amino acid residues of the amino acid sequence set out in SEQ ID NO: 10. A suitable antigen-binding fragment of SEQ ID NO: WW may comprise up to 285, up to 280, up to 275, up to 270, or up to 265 contiguous amino acid residues of the amino acid sequence set out in SEQ ID NO: 10.

An antigen-binding fragment of SEQ ID NO: 10 may comprise up to 99% of the amino acid sequence of the parent protein. For example, a suitable antigen-binding fragment of SEQ ID NO: 10 may comprise up to 98%, up to 97%, up to 96%, up to 95%, up to 94%, up to 92%, up to 91%, or up to 90% of the amino acid sequence set out in SEQ ID NO: 10.

An antigen-binding variant of SEQ ID NO: 10 may share at least 85% identity with the parent protein. For example, a suitable antigen-binding fragment of SEQ ID NO: 10 may share at least 90% identity, at least 91% identity, at least 92% identity, at least 93% identity, at least 94% identity, at least 95% identity, at least 96% identity, at least 97% identity, at least 98% identity, or at least 99% identity with the amino acid sequence set out in SEQ ID NO: 10.

An antigen-binding fragment of SEQ ID NO: 11 may comprise up to 308 contiguous amino acid residues of the parent protein. For example, a suitable antigen-binding fragment of SEQ ID NO: 11 may comprise up to 307, up to 306, up to 305, up to 304, up to 303, up to 302, up to 301, up to 300, up to 299, up to 298, up to 297, up to 296, up to 295, up to 294, up to 293, up to 292, up to 291, or up to 290 contiguous amino acid residues of the amino acid sequence set out in SEQ ID NO: 11. A suitable antigen-binding fragment of SEQ ID NO: 11 may comprise up to 285, up to 280, up to 275, up to 270, or up to 265 contiguous amino acid residues of the amino acid sequence set out in SEQ ID NO: 11.

An antigen-binding fragment of SEQ ID NO: 11 may comprise up to 99% of the amino acid sequence of the parent protein. For example, a suitable antigen-binding fragment of SEQ ID NO: 11 may comprise up to 98%, up to 97%, up to 96%, up to 95%, up to 94%, up to 92%, up to 91%, or up to 90% of the amino acid sequence set out in SEQ ID NO: 11.

An antigen-binding variant of SEQ ID NO: 11 may share at least 85% identity with the parent protein. For example, a suitable antigen-binding fragment of SEQ ID NO: 11 may share at least 90% identity, at least 91% identity, at least 92% identity, at least 93% identity, at least 94% identity, at least 95% identity, at least 96% identity, at least 97% identity, at least 98% identity, or at least 99% identity with the amino acid sequence set out in SEQ ID NO: 11.

In a suitable embodiment, the KRAS-specific endogenous targeting portion is capable of binding to both mutant KRAS and wild-type KRAS. The DARPins K19 (SEQ ID NO: 6) and K13 (SEQ ID NO: 8) and anti-KRAS scFvs of SEQ ID NOs: 10 and 11 are each examples of such KRAS-specific endogenous targeting portions.

The inventors have found that chimaeric proteins of the invention that incorporate a KRAS-specific endogenous targeting portion that binds to both wild-type KRAS and mutant KRAS are able to induce proteolysis of both forms of KRAS, but that surprisingly they selectively inhibit the proliferation of cells, such as cancer cells, that express mutant KRAS in vitro or in vivo, without inhibiting proliferation of cells expressing wild-type KRAS.

The KRAS bound may be KRAS expressed by a subject requiring prevention or treatment of a condition using a chimaeric protein in accordance with the invention.

Typically, the KRAS-specific endogenous targeting portion will be specific for human KRAS.

### “An NRAS-Specific Endogenous Targeting Portion”

An NRAS-specific endogenous targeting portion is a polypeptide component of a chimaeric protein of the invention that confers on the chimaeric protein the ability to bind specifically to NRAS.

An NRAS-specific endogenous targeting portion suitable for use in a chimaeric protein of the invention may be any endogenous polypeptide sequence capable of binding specifically to NRAS. The skilled person will be aware of many suitable assays by which the ability to bind specifically to NRAS may be assessed.

In a suitable embodiment, the NRAS-specific endogenous targeting portion is selected from the group consisting of: a NRAS-specific intracellular antibody; and an NRAS-specific DARPin.

In a suitable embodiment the NRAS-specific endogenous targeting portion is an NRAS-specific intracellular antibody. In a suitable embodiment the NRAS-specific endogenous targeting portion is an NRAS-specific scFV.

### “An HRAS-Specific Endogenous Targeting Portion”

An HRAS-specific endogenous targeting portion is a polypeptide component of a chimaeric protein of the invention that confers on the chimaeric protein the ability to bind specifically to HRAS.

An HRAS-specific endogenous targeting portion suitable for use in a chimaeric protein of the invention may be any endogenous polypeptide sequence capable of binding specifically to HRAS. The skilled person will be aware of many suitable assays by which the ability to bind specifically to HRAS may be assessed.

In a suitable embodiment, the HRAS-specific endogenous targeting portion is selected from the group consisting of: a HRAS-specific intracellular antibody; and an HRAS-specific DARPin.

In a suitable embodiment the HRAS-specific endogenous targeting portion is an HRAS-specific intracellular antibody. In a suitable embodiment the NRAS-specific endogenous targeting portion is an HRAS-specific scFV.

### LMO2

LMO2 is a protein also known as LIM domain only 2, RBTNL1, RBTN2, RHOM2, LIM Domain Only Protein 2, TTG2 and T-cell Translocation Protein 2. LMO2 is activated by chromosomal translocations t(11;14)(p13;q11) and t(7;11)(q35;p13) in T-ALL, and by changes in control regions for transcription. Furthermore, LMO2 is overexpressed in more than 50% of T-ALL cases, and is not expressed in normal T cells, thus it is considered a specific marker of lymphoblasts associated with T-ALL, and a suitable target protein to be used in the prevention or treatment of T-ALL.

Expression of LMO2 is also associate with a number of other conditions that may require prevention or treatment, including LMO2⁺ breast cancer; LMO2⁺ prostate cancer; and LMO2⁺ diffuse large B cell lymphoma.

The amino acid sequence of human wild-type LMO2 is set out in SEQ ID NO: 12.

### “An LMO2-Specific Endogenous Targeting Portion”

An LMO2-specific endogenous targeting portion is a polypeptide component of a chimaeric protein of the invention that confers on the chimaeric protein the ability to bind specifically to LMO2.

An LMO2-specific endogenous targeting portion suitable for use in a chimaeric protein of the invention may be any endogenous polypeptide sequence capable of binding specifically to LMO2. The skilled person will be aware of many suitable assays by which the ability to bind specifically to LMO2 may be assessed.

In a suitable embodiment, the LMO2-specific endogenous targeting portion is selected from the group consisting of: a LMO2-specific intracellular antibody; and an LMO2-specific DARPin.

An example of an LMO2-specific intracellular antibody suitable for use as an LMO2-specific endogenous targeting portion in accordance with the present invention is set out in SEQ ID NO: 13. This intracellular antibody is also referred to as VH576 elsewhere within this specification.

The LMO2 specific intracellular antibody of SEQ ID NO: 13 represents a suitable parent protein from which fragments or variants may be generated, as discussed elsewhere in the specification. In a suitable embodiment, an LMO2-specific endogenous targeting portion comprises the amino acid sequence set out in SEQ ID NO: 13; an LMO2-binding variant of SEQ ID NO:13; or an LMO2-binding fragment of SEQ ID NO: 13 or its variants.

An LMO2-specific endogenous targeting portion suitable for use in a chimaeric protein of the invention may share at least 85% identity with the amino acid sequence of SEQ ID NO: 13.

In a suitable embodiment, the LMO2-specific endogenous targeting portion comprises the amino acid sequence set out in SEQ ID NO: 13; or an LMO2-binding fragment thereof.

In a suitable embodiment, the LMO2-specific endogenous targeting portion consists of the amino acid sequence set out in SEQ ID NO: 13.

As discussed elsewhere in the specification, chimaeric proteins of the invention comprising the LMO2-specific endogenous targeting portion of SEQ ID NO: 13 may comprise this sequence (or its fragments or variants) in combination with a ubiquitin ligase domain comprising a VHL E3 ligase domain.

The exemplary chimaeric protein of the invention “VHL-VH576” (SEQ ID NO: 14) represents an example of a chimaeric protein comprising an LMO2-specific endogenous targeting portion based upon SEQ ID NO: 13 and a ubiquitin ligase domain comprising a VHL E3 ligase domain.

### “A Ubiquitin Ligase Domain”

The ubiquitin ligase domain is a polypeptide component of a chimaeric protein of the invention that has ubiquitin ligase activity. The presence of this domain within the chimaeric protein, along with the KRAS-specific endogenous targeting portion enables the ubiquitin ligase activity to be directed specifically to KRAS, for example cellular KRAS. In turn, this increases targeting of KRAS to the proteasome.

There are a very large number of ubiquitin ligase domains known to the skilled person. These include the VHL E3 ligase domain described above, and the UBOX domain of carboxyl-terminus of Hsc70 interacting protein (CHIP) E3 ligase. Merely by way of example the ubiquitin ligase domain of a chimaeric protein of the invention may be selected from the group consisting of: a VHL E3 ligase domain, or a fragment or variant thereof having ubiquitin ligase activity; and a UBOX domain of CHIP, or a fragment or variant thereof having ubiquitin ligase activity. The skilled person will be aware of many suitable assays by which ubiquitin ligase activity may be assessed.

The inventors have found that it is particularly suitable to use the VHL E3 ligase domain, or a fragment or variant thereof having ubiquitin ligase activity, as a ubiquitin ligase domain in the chimaeric proteins of the invention. As described further in the Examples below, chimaeric proteins of the invention incorporating a VHL E3 ligase domain have proven to be more effective than those comprising other comparator ubiquitin ligase domains.

The amino acid sequence of the VHL E3 ligase is set out in SEQ ID NO: 15, and DNA encoding the VHL E3 ligase is set out in SEQ ID NO: 16. The VHL E3 ligase domain represents a suitable parent protein from which fragments or variants may be generated, as discussed elsewhere in the specification.

It is known that residue C162 of the VHL E3 domain is important for its binding to Elongin B/C ubiquitin ligase complex and for VHL ubiquitin ligase activity in vitro. Accordingly, a suitable fragment of the VHL E3 domain for use as a ubiquitin ligase domain in a chimaeric protein of the invention may comprise residue C162. Similarly, a suitable variant of the VHL E3 domain for use as a ubiquitin ligase domain in a chimaeric protein of the invention may retain residue C162 unsubstituted.

A ubiquitin ligase domain suitable for use in a chimaeric protein in accordance with the invention may share at least 85% identity with SEQ ID NO: 15.

In a suitable embodiment, the ubiquitin ligase domain comprises the amino acid sequence set out in SEQ ID NO: 15; or a fragment thereof having ubiquitin ligase activity.

In a suitable embodiment, the ubiquitin ligase domain consists of the amino acid sequence set out in SEQ ID NO: 15.

In a suitable embodiment the ubiquitin ligase domain of a chimaeric protein of the invention comprises the UBOX domain of CHIP, or a fragment or variant thereof.

The amino acid sequence of the UBOX domain of CHIP is set out in SEQ ID NO: 17, and DNA encoding the UBOX domain of CHIP is set out in SEQ ID NO: 18. The UBOX domain of CHIP represents a suitable parent protein from which fragments or variants may be generated for use in embodiments of the invention.

A ubiquitin ligase domain suitable for use in a chimaeric protein in accordance with the invention may share at least 85% identity with SEQ ID NO: 17.

In a suitable embodiment, the ubiquitin ligase domain comprises the amino acid sequence set out in SEQ ID NO: 17; or a fragment thereof having ubiquitin ligase activity.

In a suitable embodiment, the ubiquitin ligase domain consists of the amino acid sequence set out in SEQ ID NO: 17.

As referred to above, the skilled person is well aware of methods by which the ligase activity of a ubiquitin ligase domain suitable for use in a chimaeric protein of the invention may be assessed and quantified. Merely by way of example, this may be achieved using commercially available kits, such as those sold by Abcam.

A fragment or variant of ubiquitin ligase domain, such as a VHL E3 ligase domain or UBOX domain of CHIP, suitable for use in the proteins of the invention may possess at least 75% of the ligase activity of the parent protein as measured by a suitable ligase activity assay. For example, a suitable fragment or variant may possess at least 80%, at least 85%, at least 90%, or at least 95% of the ligase activity of the parent protein. A suitable fragment or variant may possess at least 96%, at least 97%, at least 98%, or at least 99% of the ligase activity of the parent protein. A suitable variant may even possess greater ligase activity than the parent ubiquitin ligase domain from which it is derived.

### Structure of the Chimaeric Proteins of the Invention

As mentioned above, the inventors have unexpectedly found that the orientation of the constituents within chimaeric proteins of the invention can have a dramatic impact upon their effectiveness. Surprisingly, those chimaeric proteins of the invention in which the ubiquitin ligase domain is attached to the N-terminal region of the endogenous targeting portion show greater efficacy in bringing about the clearance of their cellular target (such as KRAS, NRAS, or LMO2). This is demonstrated in the results set out in the Examples.

The inventors have found that chimaeric proteins of the invention in which the ubiquitin ligase domain is attached to the N-terminal region of the endogenous targeting portion are more effective than proteins with the reversed orientation in embodiments using the VHL E3 ligase domain and in embodiments using the UBOX domain of CHIP. This difference in effectiveness is particularly pronounced in the case of chimaeric proteins of the invention comprising the VHL E3 ligase domain.

Accordingly, it is a preferred embodiment of the invention that, when the ubiquitin ligase domain is a VHL E3 ligase domain, or a fragment or variant thereof, that the ubiquitin ligase domain is attached to the N-terminal region of the endogenous targeting portion within a chimaeric protein of the invention.

Without wishing to be bound by any hypothesis, the inventors believe that this orientation of constituents within the chimaeric proteins of the invention advantageously improves accessibility to free lysine, and thereby increases the biological activity of the chimaeric protein of the invention. Furthermore, this arrangement may reduce steric hindrance between the endogenous targeting portion, the ligase domain and the target protein. This may be particularly important in the case of proteins of the invention targeting RAS, as this protein is membrane bound within the cell.

The increased effectiveness of chimaeric proteins of the first aspect of the invention having this structure is particularly pronounced in respect of the reduction of intracellular KRAS—whether this is achieved by a chimaeric protein incorporating a KRAS-specific endogenous targeting portion or a chimaeric protein incorporating a pan RAS-specific endogenous targeting portion. Accordingly, it is a preferred embodiment of the invention that a chimaeric protein comprises a VHL E3 ligase domain attached to the N-terminal region of a KRAS-specific endogenous targeting portion.

It is another preferred embodiment of the invention that a chimaeric protein comprises a VHL E3 ligase domain attached to the N-terminal region of a pan RAS-specific endogenous targeting portion. Such chimaeric proteins of the invention are particular effective in the reduction of intracellular levels of NRAS and/or KRAS.

In the case of chimaeric proteins of the second aspect of the invention, the results set out in the Examples indicate that the orientation of elements of the protein is particularly important in respect of chimaeric proteins comprising an anti-LMO2 intracellular antibody of SEQ ID NO: 13 and a VHL E3 domain. In connection with this, it can be seen that the chimaeric protein of the invention “VHL-VH576” (SEQ ID NO: 14) is effective in causing degradation of intracellular LMO2, while the protein VH576-VHL, comprising the same constituents in the reverse orientation, is not effective.

The ubiquitin ligase domain and the endogenous targeting portion may be indirectly attached to one another by a linker sequence. For example, the C-terminal region of the ubiquitin ligase domain may be attached to a linker sequence, which in turn is attached to the N-terminal region of the endogenous targeting portion. This arrangement appears particularly beneficial in chimaeric proteins of the invention comprising a KRAS-specific endogenous targeting portion.

A suitable linker sequence may comprise a plurality of glycine residues. Merely by way of example, a suitable linker sequence may comprise between three and seven glycine residues, for example four glycine residues. A suitable linker sequence may comprise a combination of glycine and serine residues. For example, a suitable linker sequence may comprise a plurality of glycine residues and a single serine residue, such as GGGGS (SEQ ID NO: 19).

An Example of such a linker sequence is found in the exemplary chimaeric protein of the invention set out in SEQ ID NO: 20. Here the linker sequence used has the amino acid sequence of SEQ ID NO: 19, and constitutes amino acid residues 216 to 220 of the exemplary chimaeric protein of SEQ ID NO: 20.

Alternatively, the ubiquitin ligase domain and endogenous targeting portion may be directly fused to one another. For example, the C-terminal region of the ubiquitin ligase domain may be fused directly to the N-terminal region of the endogenous targeting portion.

Suitably the ubiquitin ligase domain comprises only a single domain. Both the VHL E3 ligase domain and the UBOX domain of CHIP may be utilised in such embodiments (as may their single domain fragments or variants).

Suitably the endogenous targeting portion comprises only a single domain. DARPins and intracellular antibodies both constitute examples of endogenous targeting portions with single domains that may be used in such embodiments.

Suitably both the ubiquitin ligase domain and the endogenous targeting portion each comprise only a single domain. The exemplary chimaeric protein of the invention set out in SEQ ID NO: 2 represents an example of a chimaeric protein of the invention in accordance with this embodiment.

Chimaeric proteins in accordance with these embodiments of the invention, in which either the ubiquitin ligase domain, the endogenous targeting portion, or both of these components each comprise only a single domain offer a number of advantages. Examples of such advantages include the relative ease with which such chimaeric proteins can be expressed at relatively high levels, the high efficiency with which such chimaeric proteins can be isolated once expressed, and the beneficial solubility of the proteins. It will be appreciated that these benefits are particularly applicable to embodiments of the chimaeric proteins of the invention in which both the ubiquitin ligase domain and the endogenous targeting portion each comprise only a single domain.

### Exemplary Chimaeric Proteins of the Invention

Many chimaeric proteins of the invention, whether in accordance with the first aspect of the invention or the second aspect of the invention, are discussed in this specification.

The amino acid sequence of an exemplary chimaeric protein of the first aspect of the invention referred to herein as “VHL-DP KRAS” is set out in SEQ ID NO: 20. This protein comprises a VHL E3 ubiquitin ligase domain connected via a linker sequence to the N-terminal region of a K19 DARPin KRAS-specific endogenous targeting portion. A DNA sequence encoding the chimaeric protein of SEQ ID NO: 20 is set out in SEQ ID NO: 21.

The amino acid sequence of an exemplary chimaeric protein of the first aspect of the invention referred to herein as “VHL-K13” is set out in SEQ ID NO: 22. This protein comprises a VHL E3 ubiquitin ligase domain connected via a linker sequence to the N-terminal region of a K13 DARPin KRAS-specific endogenous targeting portion. A DNA sequence encoding the chimaeric protein of SEQ ID NO: 22 is set out in SEQ ID NO: 23.

The amino acid sequence of an exemplary chimaeric protein of the first aspect of the invention referred to herein as “UBOX-DP KRAS” is set out in SEQ ID NO: 24. This protein comprises a UBOX domain of CHIP connected via a linker sequence to the N-terminal region of a K19 DARPin KRAS-specific endogenous targeting portion. A DNA sequence encoding the chimaeric protein of SEQ ID NO: 24 is set out in SEQ ID NO: 25.

The amino acid sequence of an exemplary chimaeric protein of the first aspect of the invention referred to herein as “VHL-P2-E2” is set out in SEQ ID NO: 26. This protein comprises a VHL E3 ubiquitin ligase domain connected via a linker sequence to the N-terminal region of an anti-KRAS P2-E2 intracellular scFv as a KRAS-specific endogenous targeting portion.

The amino acid sequence of an exemplary chimaeric protein of the first aspect of the invention referred to herein as “VHL-P2-F3” is set out in SEQ ID NO: 27. This protein comprises a VHL E3 ubiquitin ligase domain connected via a linker sequence to the N-terminal region of an anti-KRAS P2-F3 intracellular scFv as a KRAS-specific endogenous targeting portion.

The amino acid sequence of an exemplary chimaeric protein of the first aspect of the invention referred to herein as “UBOX-P2-E2” is set out in SEQ ID NO: 28. This protein comprises a UBOX domain of CHIP connected via a linker sequence to the N-terminal region of an anti-KRAS P2-E2 intracellular scFv as a KRAS-specific endogenous targeting portion.

The amino acid sequence of an exemplary chimaeric protein of the first aspect of the invention referred to herein as “UBOX-P2-F3” is set out in SEQ ID NO: 29. This protein comprises a UBOX domain of CHIP connected via a linker sequence to the N-terminal region of an anti-KRAS P2-F3 intracellular scFv as a KRAS-specific endogenous targeting portion.

The amino acid sequence of an exemplary chimaeric protein of the first aspect of the invention referred to herein as “P2-E2-VHL” is set out in SEQ ID NO: 30. This protein comprises a VHL E3 ubiquitin ligase domain connected via a linker sequence to the C-terminal region of an anti-KRAS P2-E2 intracellular scFv as a KRAS-specific endogenous targeting portion.

The amino acid sequence of an exemplary chimaeric protein of the first aspect of the invention referred to herein as “P2-F3-VHL” is set out in SEQ ID NO: 31. This protein comprises a VHL E3 ubiquitin ligase domain connected via a linker sequence to the C-terminal region of an anti-KRAS P2-F3 intracellular scFv as a KRAS-specific endogenous targeting portion.

The amino acid sequence of an exemplary chimaeric protein of the first aspect of the invention referred to herein as “P2-E2-UBOX” is set out in SEQ ID NO: 32. This protein comprises a UBOX domain of CHIP connected via a linker sequence to the C-terminal region of an anti-KRAS P2-E2 intracellular scFv as a KRAS-specific endogenous targeting portion.

The amino acid sequence of an exemplary chimaeric protein of the first aspect of the invention referred to herein as “P2-F3-UBOX” is set out in SEQ ID NO: 33. This protein comprises a UBOX domain of CHIP connected via a linker sequence to the C-terminal region of an anti-KRAS P2-F3 intracellular scFv as a KRAS-specific endogenous targeting portion.

The amino acid sequence of an exemplary chimaeric protein of the first aspect of the invention referred to herein as “UBOX-iDAb RAS” is set out in SEQ ID NO: 5. This protein comprises a UBOX domain connected via a linker sequence to the N-terminal region of an anti-pan RAS intracellular single domain antibody as a pan RAS-specific endogenous targeting portion.

The amino acid sequence of an exemplary chimaeric protein of the first aspect of the invention referred to herein as “iDAb RAS-UBOX” is set out in SEQ ID NO: 34. This protein comprises a UBOX domain connected via a linker sequence to the C-terminal region of an anti-pan RAS intracellular single domain antibody as a pan RAS-specific endogenous targeting portion. As demonstrated in the Examples, this chimaeric protein of the invention is actually more effective in reducing intracellular levels of RAS (whether KRAS, HRAS or NRAS) than the UBOX-iDAb RAS protein referred to above.

The amino acid sequence of an exemplary chimaeric protein of the first aspect of the invention referred to herein as “VHL-iDAb RAS” is set out in SEQ ID NO: 35. This protein comprises a VHL E3 ubiquitin ligase domain connected via a linker sequence to the N-terminal region of an anti-pan RAS intracellular single domain antibody, as a pan RAS-specific endogenous targeting portion.

The amino acid sequence of an exemplary chimaeric protein of the second aspect of the invention referred to herein as “VHL-VH576” is set out in SEQ ID NO: 14. This protein comprises a VHL E3 ubiquitin ligase domain connected via a linker sequence to the N-terminal region of the anti-LMO2 scFv VH576, as an anti-LMO2 endogenous targeting portion.

In a suitable embodiment a chimaeric protein of the invention may share at least 80% or at least 85% identity with amino acid sequences of the exemplary chimaeric proteins set out in any of SEQ ID NOs: 20; 4; 5; 14; 22; 24; 26; 27; 28; 29; 30; 31; 32; 33; 34; or 35. Suitably such a chimaeric protein of the invention may share at least 86%, at least 87%, at least 88%, at least 89%, at least 90%, at least 91%, at least 92%, at least 93%, at least 94%, at least 95%, at least 96%, at least 97%, at least 98%, or at least 99% identity with the amino acid sequences of the exemplary chimaeric proteins set out in any of SEQ ID NOs: 20; 4; 5; 14; 22; 24; 26; 27; 28; 29; 30; 31; 32; 33; 34; or 35.

A chimaeric protein of the invention may comprise the amino acid sequence set out in any of SEQ ID NOs: 20; 4; 5; 14; 22; 24; 26; 27; 28; 29; 30; 31; 32; 33; 34; or 35.

A chimaeric protein of the invention may consist of the amino acid sequence set out in any of SEQ ID NOs: 20; 4; 5; 14; 22; 24; 26; 27; 28; 29; 30; 31; 32; 33; 34; or 35.

It will be noted that the amino acid sequences provided in respect of the exemplary chimaeric proteins of the invention of SEQ ID NOs: 20; 22; and 24 incorporate a sequence “VDGGS” (SEQ ID NO: 40). Furthermore, the amino acid sequences provided in respect of the exemplary chimaeric proteins of the invention of SEQ ID NOs: 20; 5; 22; 24 and 35, and the anti-KRAS scFvs of the invention of SEQ ID NOs: 10 and 11, incorporate a sequence “DYKDDDDK” (SEQ ID NO: 41). SEQ ID NO: 40 is generated as a result of restriction enzymes used in the preparation of the chimaeric proteins of the invention, and SEQ ID NO: 41 is a FLAG tag used for detection of the chimaeric proteins. It will be appreciated that a fragment or variant of a chimaeric protein or scFv of the invention may lack one or both of SEQ ID NO: 40 and SEQ ID NO: 41, without any substantial impact upon its ability to reduce intracellular levels of RAS (in the case of a chimaeric protein of the first aspect of the invention) or bind to KRAS (in the case of an scFV of the invention). In particular, a fragment or variant of any of the exemplary proteins set out in SEQ ID NOs: 20; 5; 10; 11; 22; 24; or 35 may lack one or both of SEQ ID NO: 40 and SEQ ID NO: 41.

### A Nucleic Acid Molecule of the Invention

The third aspect of the invention provides a nucleic acid molecule comprising a nucleic acid sequence encoding a chimaeric protein of the invention. This aspect also provides a cell comprising such a nucleic acid molecule. The protein encoded may be a protein in accordance with the first aspect of the invention, or a protein in accordance with the second aspect of the invention, and may be in accordance with any of the embodiments of these aspects described herein.

Suitably a nucleic acid molecule of the invention may comprise DNA. A DNA sequence encoding the exemplary chimaeric protein of the first aspect of the invention set out in SEQ ID NO: 20 is provide in SEQ ID NO: 21.

Suitably a nucleic acid molecule of the invention may comprise RNA.

A nucleic acid molecule of the invention may be provided in the form of a vector comprising the nucleic acid molecule. Such a vector may be expressed by a cell to produce a chimaeric protein of the invention.

Merely by way of example, a vector in accordance with such an embodiment may be a lentivirus vector, as considered further in the Examples.

A cell comprising a nucleic acid in accordance with the invention may be used to produce a chimaeric protein in accordance with the invention. Accordingly, in a further aspect, the invention provides a method of producing a chimaeric protein in accordance with a first aspect of the invention, the method comprising: providing a cell comprising a nucleic in accordance with the third aspect of the invention, and maintaining the cell under conditions such that it produces a chimaeric protein in accordance with the first aspect of the invention.

As demonstrated in the Examples, nucleic acids in accordance with the third aspect of the invention represent suitable agents that may be provided to cancer cells, such as cancer cells of tumours, in order to bring about the treatment of cancer.

### Methods of Treatment and Medical Uses

The fifth aspect of the invention provides a method of preventing or treating a RAS-associated disorder, the method comprising providing a therapeutically effective amount of a chimaeric protein of the first aspect of the invention to a subject in need thereof.

The RAS-associated disorder may be selected from the group consisting of: a RAS-associated cancer; a RAS-associated psychiatric disorder; and a RASopathy.

A RAS-associated cancer may be associated with a mutation in a RAS isoform. In such an embodiment, an appropriate chimaeric protein of the invention for use in such treatment may be selected by virtue of the specificity of the endogenous targeting portion (for example, either using a pan RAS-specific endogenous targeting portion or an endogenous targeting portion specific for the isoform of RAS associated with the mutation).

The fifth aspect of the invention also provides a chimaeric protein, nucleic acid, or pharmaceutical composition in accordance with the first, third or fourth aspect of the invention, for use as a medicament in the prevention or treatment of a disorder. Suitably the disorder is selected from: a RAS-associated cancer and a RASopathy.

In a suitable embodiment, the RAS-associated cancer is selected from: RAS-associated lung cancer; RAS-associated pancreatic cancer; RAS-associated colorectal cancer; adrenocortical carcinoma; bladder urothelial carcinoma; breast invasive carcinoma; cervical squamous cell carcinoma or endocervical adenocarcinoma; cholangiocarcinoma; colon adenocarcinoma; lymphoid neoplasm diffuse large B-cell lymphoma; oesophageal carcinoma; glioblastoma multiforme; head and neck squamous cell carcinoma; kidney chromophobe; kidney renal clear cell carcinoma; kidney renal papillary cell carcinoma; acute myeloid leukaemia; brain lower grade glioma; liver hepatocellular carcinoma; lung adenocarcinoma; lung squamous cell carcinoma; ovarian serous cystadenocarcinoma; pancreatic adenocarcinoma; pheochromocytoma or paraganglioma; prostate adenocarcinoma; rectum adenocarcinoma; sarcoma; skin cutaneous melanoma; stomach adenocarcinoma; testicular germ cell tumours; thyroid carcinoma; thymoma; uterine corpus endometrial carcinoma; uterine carcinosarcoma; and uveal melanoma.

Suitably a RASopathy to be prevented or treated is selected from: capillary malformation-av malformation syndrome; autoimmune lymphoproliferative syndrome; cardiofaciocutaneous syndrome; hereditary gingival fibromatosis type 1; neurofibromatosis type 1; Noonan syndrome; Costello syndrome; and Legius syndrome.

The sixth aspect of the invention provides a method of preventing or treating a condition associated with expression of LMO2, the method comprising providing a therapeutically effective amount of a chimaeric protein according to the second aspect of the invention to a subject in need thereof. The condition associated with expression of LMO2 may be selected from the group consisting of: T-ALL; LMO2⁺ breast cancer; LMO2⁺ prostate cancer; and LMO2⁺ diffuse large B cell lymphoma.

The sixth aspect of the invention also provides a chimaeric protein, nucleic acid, or pharmaceutical composition in accordance with the second, third or fourth aspect of the invention, for use as a medicament in the prevention or treatment of a condition associated with expression of LMO2, such as T-ALL.

### “Providing” a Chimaeric Protein of the Invention

It will be recognised that the chimaeric proteins of the invention may be “provided” as required (for example, to a subject or to cells) by administration of the protein itself, suitably incorporated in a pharmaceutical composition of the invention.

It will be appreciated that the chimaeric proteins of the invention (whether in accordance with the first or second aspects of the invention) achieve their therapeutic effects via intracellular actions. Accordingly, in a suitable embodiment a chimaeric protein of the invention may be adapted in order to promote entry of the chimaeric protein into cells. Suitable adaptations may include, but are not limited to, those selected from the group consisting of: addition of protein transduction domains; and addition of internalising immunoglobulin g (IgG) sequences. Suitable protein transduction domains may be selected from the group consisting of: Antennapedia peptide; and HIV TAT peptide.

However, it will also be appreciated that the chimaeric proteins of the invention may also be provided to a subject by means of administration, and subsequent expression, of a nucleic acid molecule in accordance with the third aspect of the invention. Such expression may be permanent expression. Alternatively, the expression may be transient expression sufficient to provide a therapeutically effective amount of a chimaeric protein of the invention.

Suitably a nucleic acid for use to provide a chimaeric protein of the invention may be mRNA. Suitably a nucleic acid for use to provide a chimaeric protein of the invention may be provided in the form of a vector or plasmid. Suitably such a vector may be a viral vector.

### Therapeutic Agents of the Invention, and Therapeutically Effective Amounts

A “therapeutic agent” of the invention may be a chimaeric protein of the invention, a nucleic acid molecule of the invention, or a pharmaceutical composition of the invention (comprising such a chimaeric protein or nucleic acid molecule)

A “therapeutically effective amount” of a therapeutic agent of the invention is an amount sufficient to delay, inhibit, or alleviate either clinical symptoms or progression of a disorder to be treated.

A therapeutically effective amount of a therapeutic agent of the invention may be provided in a single incidence of administration. Alternatively, a therapeutically effective amount of a therapeutic agent of the invention may be provided by means of multiple incidences of administration.

A medical use or method of treatment in accordance with the fifth aspect of the invention may be initiated after diagnosis of a subject as having a RAS-associated disorder (such as a RAS-associated cancer or a RASopathy). Alternatively, a medical use or method of treatment in accordance with the fifth aspect of the invention may be initiated in respect of a subject having symptoms consistent with having a RAS-associated disorder (such as a RAS-associated cancer or a RASopathy).

In the same manner, a medical use or method of treatment in accordance with the sixth aspect of the invention may be initiated after diagnosis of a subject as having a condition associated with expression of LMO2, such as T-ALL. Alternatively, a medical use or method of treatment in accordance with the sixth aspect of the invention may be initiated in respect of a subject having symptoms consistent with having a condition associated with expression of LMO2, such as T-ALL.

### Further Uses of the Chimaeric Proteins of the Invention

In another aspect, the invention provides a method of killing cancer cells expressing a mutant form of RAS, the method comprising contacting the cancer cells with an effective amount of a chimaeric protein of the first aspect of the invention.

In a further aspect, the invention provides a method of reducing the size of a RAS-associated tumour, the method comprising contacting cells of the tumour with an effective amount of a chimaeric protein of the first aspect of the invention.

In a still further aspect, the invention provides a method of reducing intracellular RAS in a cell, the method comprising contacting the cell with an effective amount of a chimaeric protein of the first aspect of the invention.

In a still further aspect, the invention provides a method of killing cancer cells that express LMO2, the method comprising contacting the cancer cells with an effective amount of a chimaeric protein of the second aspect of the invention. The cancer cells that express LMO2 may be associated with T-ALL, with LMO2⁺ breast cancer, with LMO2⁺ prostate cancer, or with LMO2⁺ diffuse large B cell lymphoma.

The chimaeric protein of the invention may be provided by administration of the protein, or by administration of a nucleic acid molecule of the invention. Either a protein of the invention or a nucleic acid molecule of the invention may be provided by means of a pharmaceutical composition of the invention.

### “Pharmaceutical Compositions of the Invention”

The fourth aspect of the invention provides a pharmaceutical composition comprising a chimaeric protein of the invention and/or a nucleic acid encoding a chimaeric protein of the invention and a pharmaceutically acceptable carrier.

Nanoparticles represent a suitable example of a pharmaceutically acceptable carrier that may be used in the pharmaceutical compositions of the invention.

The protein may be a protein in accordance with the first aspect of the invention (which is to say the pharmaceutical composition may comprise a chimaeric protein comprising a ubiquitin ligase domain and a KRAS-specific endogenous targeting portion and/or a nucleic acid molecule comprising a nucleic acid sequence encoding a chimaeric protein comprising a ubiquitin ligase domain and a KRAS-specific endogenous targeting portion).

Alternatively, the protein may be a protein in accordance with the second aspect of the invention (which is to say the pharmaceutical composition may comprise a chimaeric protein comprising a ubiquitin ligase domain and an LMO2-specific endogenous targeting portion and/or a nucleic acid molecule comprising a nucleic acid sequence encoding a chimaeric protein comprising a ubiquitin ligase domain and an LMO2-specific endogenous targeting portion).

An appropriate chimaeric protein and/or nucleic acid molecule of the invention to be incorporated in a pharmaceutical composition may be determined with respect to the medical use to which the pharmaceutical composition is to be put (in keeping with the discussions of medical uses elsewhere in the specification).

A pharmaceutical composition of the invention may be formulated for use by any desired route of administration.

Suitably, the pharmaceutical composition may be formulated for injection. For instance, the pharmaceutical composition may be formulated for intravenous administration, such as intravenous infusion. Such a pharmaceutical composition may comprise pharmaceutically acceptable excipients, buffers, and the like, as is conventional in the art.

The pharmaceutical composition may be in lyophilized form.

The pharmaceutical composition of the present invention may optionally comprise a pharmaceutically acceptable additive, in addition to the chimaeric protein or nucleic acid molecule of the present invention or a pharmaceutically acceptable salt thereof and/or a carrier as described above. Examples of such an additive include an emulsifier aid (e.g., a fatty acid containing 6 to 22 carbon atoms or a pharmaceutically acceptable salt thereof, albumin, dextran), a stabilizing agent (e.g., cholesterol, phosphatidic acid), an isotonizing agent (e.g., sodium chloride, glucose, maltose, lactose, sucrose, trehalose), and a pH adjuster (e.g., hydrochloric acid, sulfuric acid, phosphoric acid, acetic acid, sodium hydroxide, potassium hydroxide, triethanolamine). These additives may be used either alone or in combination. The content of the additive(s) in the pharmaceutical composition of the present invention is reasonably 90% by weight or less, such as 60% by weight or less, and suitably 50% by weight or less.

The pharmaceutical composition of the present invention may be prepared by adding the compound (e.g. chimaeric protein or nucleic acid molecule) of the present invention or a pharmaceutically acceptable salt thereof to a dispersion of a carrier, followed by adequate stirring. The additive(s) may be added at any appropriate stage, either before or after adding the compound of the present invention or a pharmaceutically acceptable salt or hydrate thereof. Any aqueous solvent may be used for adding the compound of the present invention or a pharmaceutically acceptable salt or hydrate thereof as long as it is pharmaceutically acceptable, and examples include injectable water, injectable distilled water, electrolytic solutions (e.g., physiological saline), and sugar solutions (e.g., glucose solution, maltose solution). Moreover, in this case, conditions including pH and temperature may be selected as appropriate by those skilled in the art.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents and carriers (hereby incorporated by reference). Suitable dosages, formulations, administration routes, compositions, dosage forms, combinations with other therapeutic agents, pro-drug formulations are also provided in WO2007/031091.

The pharmaceutical composition of the present invention may be formulated into a solution or a lyophilized formulation thereof. Such a lyophilized formulation may be prepared in a standard manner by freeze-drying the pharmaceutical composition of the present invention in a solution form. For example, the pharmaceutical composition of the present invention in a solution form may be sterilized as appropriate (e.g. by conventional sterilization or sterile filtration techniques) and then dispensed in given amounts into vial bottles, followed by preliminary freezing under conditions of about −40° C. to −20° C. for about 2 hours, primary drying at about 0° C. to 10° C. under reduced pressure and then secondary drying at about 15° C. to 25° C. under reduced pressure. Moreover, in most cases, the vials may be purged with a nitrogen gas and then capped, thereby giving a lyophilized formulation of the pharmaceutical composition of the present invention.

Such a lyophilized formulation of the pharmaceutical composition of the present invention may generally be used after being reconstituted by addition of any appropriate solution (i.e., a reconstituting solution). Examples of such a reconstituting solution include injectable water, physiological saline, and other commonly used infusion solutions. The volume of such a reconstituting solution will vary, e.g., depending on the intended use and is not limited in any way, but it is reasonably 0.5- to 2-fold greater than the solution volume before freeze-drying, or 500 mL or less.

The pharmaceutical composition of the present invention may be administered in any pharmaceutically acceptable mode, which may be selected as appropriate for the intended therapeutic method. Suitable methods include intravenous administration, intraarterial administration, intramuscular administration, subcutaneous administration, oral administration, interstitial administration, percutaneous administration and so on. Moreover, the composition of the present invention may be in any dosage form, and examples include various types of injections, oral formulations, drops, inhalants, ointments, lotions, etc.

### EXAMPLES

### Example 1—Chimaeric Proteins in Accordance with the First Aspect of the Invention

### 1 Background

The following study was undertaken to demonstrate the effectiveness of therapeutic agents of the invention, to investigate their mode of action, and to compare their effectiveness to suitable control agents.

As will be seen, the inventors found that chimaeric proteins and nucleic acid molecules in accordance with the present invention have the ability to inhibit cancer cell proliferation in vitro, and to bring about a reduction in human tumours in vivo. The inhibitory effects of the therapeutic agents of the invention are selective for cells expressing mutant forms of RAS.

Accordingly, the chimaeric proteins and nucleic acid molecules of the invention represent promising new therapeutic agents for use in the prevention and/or treatment of RAS-associated cancers, or RASopathies.

### 2 Methods

### 2.1 Cell Culture

A549, H358, HCT116, HEK293T, HT1080, MIA PaCa2 cells were grown in DMEM medium (Life Technologies), H1299, HCC827, T24 cells in RPMI medium (Life Technologies) and MRC5 in MEMα medium (Life Technologies). All cell lines were supplemented with 10% FBS (Sigma) and 1% Penicillin/Streptomycin (Life Technologies). Cells were grown at 37° C. with 5% CO₂. Characterisation of the cell lines was achieved by cloning and sequencing of the K, N and HRAS cDNA from each line. MIA PaCa2 was authenticated by short-tandem repeat (STR) DNA profiling services (ATTC). All cell lines were tested to confirm that they were free of mycoplasma.

### 2.2 Cell Transfection

HEK293T cells were seeded in 6 well plates (650,000 cells per well) and 2 μg of plasmid DNA was transfected with Lipofectamine 2000 (Thermo-Fisher, see also BRET2 methods section below). HCT116 cells were seeded in 6 well plates (650,000 cells per well). Cells were transfected after 24 hours later with 2.5 μg of plasmid, 8.75 μL of Lipofectamine LTX and 2.5 g of PLUS™ Reagent (Thermo-Fisher) for another 24 hours before Western blot analysis.

### 2.3 Molecular Cloning

All RAS cDNAs (mutants KRAS, KRAS^{WT}, NRAS^{Q61H} and HRAS^{G12V}-CAAX) were cloned into the pEF-RLuc8-MCS, pEF-GFP²-MCS or pEF-3×FLAG-MCS plasmids, full-length CRAF^{S257L} was cloned into pEF-GFP²-MCS and DARPin were cloned into the pEF-MCS-GFP² or pEF-MCS-mCherry plasmid. The cloning details of all these plasmids are described elsewhere (Bery, N., Cruz-Migoni, A., Bataille, C. J., Quevedo, C. E., Tulmin, H., Miller, A., Russell, A., Phillips, S. E., Carr, S. B., and Rabbitts, T. H. (2018). “BRET-based RAS biosensors that show a novel small molecule is an inhibitor of RAS-effector protein-protein interactions”; Bery, N., Legg, S., Debreczeni, J., Breed, J., Embrey, K., Stubbs, C., Kolasinska-Zwierz, P., Barrett, N., Marwood, R., Watson, J., et al. (2019). “KRAS-specific inhibition using a DARPin binding to a site in the allosteric lobe”. Nat Commun 10, 2607.; Bery, N., and Rabbitts, T. H. (2019). “Bioluminescence Resonance Energy Transfer 2 (BRET2)-Based RAS Biosensors to Characterize RAS Inhibitors”. Curr Protoc Cell Biol).

Full-length VHL and UBOX domain (amino acids 128-303 from the CHIP E3 ligase) were cloned into Pmll/Xhol sites of the pEF-GFP²-MCS or into Notl/Xbal sites of the pEF-MCS-GFP² plasmids to replace the GFP² moiety. DARPins and iDAbs were inserted into pEF-VHL-MCS and pEF-UBOX-MCS using Notl/Xbal sites or into pEF-MCS-UBOX and pEF-MCS-VHL using Pmll/Notl sites. A single FLAG tag was added by PCR at the carboxy terminal end of the DARPin degrader vectors or on the amino terminal end of the iDAb degrader vectors.

VHL-DP KRAS-FLAG, VHL-DP Ctl-FLAG, FLAG-iDAb RAS-UBOX and FLAG-iDAb Ctl-UBOX sequences were cloned in the TLCV2 lentivector (Addgene plasmid #87360) by PCR using Agel/Nhel sites. Coding region DNA and protein sequences of these four constructs are shown in FIG. 9.

### 2.4 Lentivirus Production

For each virus produced, 4.5×10⁶ HEK293T cells were seeded per 100 mm dish (7×100 mm dishes per virus production) in 9 mL of complete DMEM. 24 hours later, cells were transfected with 12 μg of the TLCV2 construct of interest (i.e. VHL-DP KRAS-FLAG, VHL-DP Ctl-FLAG, FLAG-iDAb RAS-UBOX and FLAG-iDAb Ctl-UBOX), 8 μg of psPAX2, 3 μg of pMD2.G (the latter are lentiviral packaging and envelope vectors, respectively) and 46 μL of Lipofectamine 2000 (quantities for one 100 mm dish). The supernatants were collected 48 hours after transfection, centrifuged 5 minutes at 640×g, filtered (0.45 m filter) and centrifuged 2 hours at 48,000×g at 4° C. The virus from 7×100 mm dishes was resuspended in 250 μL of PBS.

### 2.5 Viral Transduction and Macrodrug Expression

Cells were transduced with the appropriate lentiviruses for 48 hours in 6 well-plate in 1 mL of medium containing 8 μg·mL⁻¹ of polybrene (Sigma, Cat #107689). Transduced cells were selected with puromycin (MP Biomedicals, Cat #194539). The puromycin concentrations used for selection of each cell line are shown below:

| Cell line | Puromycin concentration (μg · mL⁻¹) |
|---|---|
| H358 | 0.75 |
| MIA PaCa2 | 0.75 |
| A549 | 0.5 |
| H1299 | 0.8 |
| HT1080 | 0.75 |
| T24 | 1 |
| HCC827 | 0.5 |
| MRC5 | 0.8 |

Doxycycline (Sigma, Cat #D9891) was used to induce the expression of macrodrug E3-ligase or macrodrug GFP² fusions or controls from the TLCV2 lentivector. The doxycycline induction was carried out by addition of stock solution (100 μg·mL⁻¹) to culture medium and continued incubation at 37° C. For proteasome inhibition, epoxomicin (Sigma, Cat #E3652) was used at 0.8 μM for 18 hours followed by protein analysis.

### 2.6 Establishment of H358 and H1299-FLuc Stable Clones

H358 and H1299 cells expressing VHL-DP KRAS and iDAb RAS-UBOXwere transfected with pEF-FLuc using Lipofectamine LTX following the manufacturer recommendations. After 48 hours of transfection, cells were selected with 1 mg·mL⁻¹ of G418 (Sigma, Cat #A1720) and clones were picked and characterised.

### 2.7 Quantitative Real-Time PCR

H358 cells were plated at 0.8×10⁶ cells per well in a six-well plate. After 24 hours, the cells were treated or not with 0.2 μg·mL⁻¹ of doxycycline for 24 hours. Cell were lysed in 1 mL of TRIzol reagent (Life Technologies) per six well. Total RNA was extracted with the Direct-zol™ RNA miniprep (Zymo Research) following the manufacturer's protocol. RNA was eluted with 15 μL of nuclease-free H₂O. cDNA was synthesised from 1.5 μg of total RNA per condition using SuperScript II Reverse Transcriptase (Invitrogen). Real-time PCR was performed with 400 nM primers, diluted with 12.5 μL Fast SYBR Green Master Mix (Applied Biosystems) in a final volume of 25 μL. RT-PCR experiments were performed with the following protocol on a 7500 Fast (Applied Biosystems): 95° C. for 20 s, 40 cycles of 95° C. for 3 s, and 60° C. for 30 s. qRT-PCR samples were performed and analysed in duplicate, from two independent experiments. GAPDH was used for normalisation.

Primers used in this stud are as follows:

DUSP6For:
(SEQ ID NO: 36)
5′ CTCGGATCACTGGAGCCAAAAC 3′
DUSP6Rev:
(SEQ ID NO: 37)
5′ GTCACAGTGACTGAGCGGCTAA 3′
GAPDHFor:
(SEQ ID NO: 38)
5′ GTCTCCTCTGACTTCAACAGCG 3′
GAPDHRev:
(SEQ ID NO: 39)
5′ ACCACCCTGTTGCTGTAGCCAA 3′

### 2.8 BRET2 Assays and Measurements

For all BRET experiments (titration curves and competition assays) 650,000 HEK293T cells were seeded in each well of a 6-well plate. After 24 hours at 37° C., cells were transfected with a total of 1.6 μg of DNA mix (with donor+acceptor±competitor plasmids), using Lipofectamine 2000 transfection reagent (Thermo-Fisher). Cells were detached after 24 hours, washed with PBS and seeded in a white 96-well plate (clear bottom, PerkinElmer) in OptiMEM no phenol red medium complemented with 4% FBS. Cells were incubated for an additional 20-24 hours at 37° C. before the BRET assay reading. A detailed protocol for BRET assays has been published elsewhere.

BRET2 signal was determined immediately after injection of coelenterazine 400a substrate (10 μM final) to cells (Cayman Chemicals), using a CLARIOstar instrument (BMG Labtech) with a luminescence module.

### 2.9 2D and 3D Cell Proliferation Assays

Cells were seeded in white 96-well plates (clear bottom, PerkinElmer, Cat #6005181) for 2D-adherent proliferation assays or in ultra-low attachment 96-well plates (Corning, Cat #7007) for 3D spheroid assays. All cell seeding was optimised to maintain linear growth over the time of the assay. The following day, a 10× doxycycline solution was prepared (1-2 μg·mL⁻¹ for 0.1-0.2 μg·mL⁻¹ final concentration). Cells were incubated in the presence of the doxycycline for 6 days. Cell viability was analysed every two days using CellTiter-Glo (Promega, Cat #G7572) by incubation with the cells for 15 minutes. Cell viability was determined by normalising doxycycline-treated cells to non-treated cells. Cells from the ultra-low attachment plates were transferred into a white 96-well plate (Greiner, Cat #655075) before reading on a CLARIOstar instrument.

### 2.10 Cell Growth Assay of H358-FLuc and H1299-FLuc Clones

40,000 of H358-FLuc (iDAb RAS-UBOX or VHL-DP KRAS) or 60,000 of H1299-FLuc (iDAb RAS-UBOX or VHL-DP KRAS) cells were seeded per well of 6-well plate (each condition done in duplicate). After 24 hours, medium alone (−dox) or medium containing 0.2 μg·mL⁻¹ doxycycline (+ dox) was added in each well. Viable cells were counted with a haematocytometer and trypan blue every two days.

### 2.11 Immuno-Precipitation Assay

HEK293T cells were transfected for 24 hours with pEF-3×FLAG-KRAS^{WT} or pEF-3×FLAG-KRAS^{G12D} and pEF-DARPins-GFP² plasmids. Cells were washed once with PBS and lysed in the immuno-precipitation buffer (150 mM NaCl, 50 mM Tris-HCl pH 7.4, 10 mM MgCl₂, 10% glycerol and 0.5% Triton X-100) supplemented with protease inhibitors (Sigma, Cat #P8340) and phosphatase inhibitors (Thermo-Fisher, Cat #1862495) for 20 min. Lysates were centrifuged for 15 min and the supernatant incubated with protein G magnetic beads (Life Technologies, Cat #10004D) and anti-FLAG antibody (Sigma, Cat #F3165). The complexes were incubated for 4 hours at 4° C. with rotation. Beads were washed 5 times with the IP buffer, before the bound proteins were eluted with 1× loading buffer and resolved on 12.5% SDS-PAGE.

### 2.12 Western Blot Analysis

Cells were washed once with PBS and lysed in SDS-Tris buffer (STB: 1% SDS, 10 mM Tris-HCl pH 7.4) supplemented with protease inhibitors (Sigma) and phosphatase inhibitors (Thermo-Fisher). Cell lysates were sonicated with a Branson Sonifier.

Mouse tumours were lysed in the radioimmunoprecipitation assay (RIPA) buffer (150 mM NaCl, 1.0% Triton X-100, 0.5% sodium deoxycholate, 0.1% SDS, 50 mM Tris, pH 8.0) with a ratio of 200 μL of lysis buffer for 10 mg of tumour and homogenised with an electric disperser (T10 basic ULTRA-TURRAX, IKA) until the tissue was liquefied. The lysate was incubated on ice for 1 hour, followed by centrifugation at 16,100×g at 4° C. and the supernatant was collected. The protein concentrations from cell and tumour lysates were determined by using the Pierce BCA protein assay kit (Thermo-Fisher). Equal amounts of protein (20-50 g) were resolved on 10 or 12.5% SDS-PAGE and subsequently transferred onto a PVDF membrane (GE Healthcare). The membrane was blocked either with 10% non-fat milk (Sigma, Cat #70166) or 10% BSA (Sigma, Cat #A9647) in TBS-0.1% Tween20 and incubated overnight with primary antibody at 4° C. After washing, the membrane was incubated with horse radish peroxidase-conjugated secondary antibody for 1 hour at 20° C. The membrane was washed with TBS-0.1% Tween and developed using Clarity Western ECL Substrate (Bio-Rad) and CL-XPosure films (Thermo-Fisher) or the ChemiDoc XRS+ imaging system (Bio-Rad).

Primary antibodies include anti-phospho-p44/22 MAPK (pERK1/2) (1/4000, CST, Cat #9101S), anti-p44/42 MAPK (total ERK1/2) (1/1000, CST, Cat #9102S), anti-phospho-MEK1/2 (1/2000, CST, Cat #9154S), anti-MEK1/2 (1/500, CST, Cat #4694S), anti-phospho-AKT S473 (1/1000, CST, Cat #4058S), anti-AKT (1/1000, CST, Cat #9272S), anti-pan-RAS (1/200, Millipore, Cat #OP40), anti-KRAS (1/100, Santa Cruz Biotechnologies, Cat #sc-30), anti-NRAS (1/100, Santa Cruz Biotechnologies, Cat #sc-31 now discontinued and 1/3000, Abcam, Cat #ab77392), anti-HRAS (1/500, Proteintech, Cat #18295-1-AP), anti-cleaved PARP (1/1000, CST, Cat #9541), anti-cleaved caspase 3 (1/500, CST, Cat #9664), anti-GFP (1/500, Santa Cruz Biotechnologies, Cat #sc-9996), anti-FLAG (1/2000, Sigma, Cat #F3165), anti-β-actin (1/5000, Sigma, Cat #A1978) and anti-αtubulin (1/2000, Abcam, Cat #ab4074). Tertiary antibodies include anti-mouse IgG HRP-linked (CST, Cat #7076), anti-rabbit IgG HRP-linked (CST, Cat #7074) and anti-goat IgG HRP-linked (Santa Cruz Biotechnologies, Cat #2354).

### 2.13 Human Tumour Xenograft Assay in Nude Mice

4.5×10⁶ H358-FLuc expressing either iDAb RAS-UBOX clone B2 or VHL-DP KRAS clone F7 or 5×10⁶ H1299-FLuc expressing either iDAb RAS-UBOX clone E1 or VHL-DP KRAS clone E5 were injected subcutaneously into the left flank of 5-7-week-old female CD-1 athymic nude mice (Charles River). The mice were fed with normal diet and water until their subcutaneous tumour reached 3-4 mm diameter (2-3 mm for H1299 cells), approximately 18 days after injection. The mice were divided into two groups of 5 mice (3 mice for H1299-FLuc/VHL-DP KRAS), one of which was supplied with dox (Sigma) via drinking water (2 mg·mL⁻¹ in 20% black-currant juice) and doxycycline diet (200 mg·kg⁻¹, Special Diets Services). Note on the first day of doxycycline treatment, mice were injected with 100 μL of 4 mg·mL⁻¹ of doxycycline in sterile 0.9% aqueous NaCl by intraperitoneal injection. One mouse of each of the no doxycycline control groups injected with H358-FLuc/VHL-DP KRAS, H358-FLuc/iDAb RAS-UBOX and H1299-FLuc/VHL-DP KRAS was excluded from analysis due to lack of tumour development. Subcutaneous tumour growth was monitored by measuring three times weekly with digital calipers (Thermo-Fisher) and by bioluminescence as described below. Tumour volume was calculated using the formula: tumour volume=(L×W²)/2, in which L and W refer to the length and width of the tumour, respectively. Animals were culled in accordance with license restrictions. After humane sacrifice, the mice were dissected for tumours sampling.

### 2.14 In Vivo Bioluminescence Imaging (BLI)

Bioluminescence was measured in transplanted subcutaneous tumours non-invasively using the IVIS Lumina imaging system (PerkinElmer) following injection of the luciferase substrate D-luciferin. All of the images were taken after intraperitoneal injection of 150 μL of D-luciferin (stock solution at 30 mg·mL⁻¹ in DPBS, PerkinElmer) corresponding to 150 mg·kg⁻¹ body weight of D-luciferin. BLI were acquired after 5 min of D-luciferin injection. During image acquisition, mice were sedated continuously by inhalation of 3% isoflurane. Image analysis and bioluminescence quantification was performed using Living Image software (PerkinElmer).

### 2.15 Quantification and Statistical Analysis

Quantifications were performed using Image Lab (Biorad), Prism 8.0 (GraphPad Software) or Living Image (PerkinElmer). BRET titration curves and statistical analysis were performed using Prism 8.0 (GraphPad Software). The data are typically presented as mean±SD or SEM as specified in the figure legends. Statistical analyses were performed with an unpaired two-tail Student's t test, unless otherwise indicated in the figure legends. ns non-significant,*P<0.05, **P<0.01, ***P<0.001, ****P<0.0001.

### 3 Results

### 3.1 Engineering KRAS-Specific and Pan-RAS Degraders

The addition of warheads to intracellular antibodies and other macromolecular reagents is an implied strategy to increase efficacy, for example via the transfer of target proteins to the proteasome for degradation. Intracellular single domains have been functionalised with E3 ligase domains, such as the UBOX domain of the carboxyl terminus of Hsc70-interacting protein (CHIP) ligase, VHL or FBOX. However, there is nothing in the literature that allows predictions to be made as to which E3 ligase is applicable in specific cases nor how the different constituents in a protein should be engineered relative to one another (N or C terminal fusion with the E3 ligase). We have tested both the UBOX domain and VHL E3 ligase fused to the specific DP KRAS or the iDAb RAS. Controls comprised a mutant DARPin where the RAS-binding tryptophan repeats are mutated into glycine and alanine residues (herein DP Ctl) or a non-relevant iDAb (herein iDAb Ctl). All the proteins were engineered with either N or C-terminal fusions with each E3 ligase. Accordingly, DP Ctl does not bind to KRAS (mutant and WT) as shown by BRET donor saturation assays (FIG. 9a) and by co-immunoprecipitation (FIG. 9b). Furthermore, like the negative control DARPin E3.5, the DP Ctl mutant does not inhibit mutant KRAS/CRAF^{FL} interaction (FIG. 9c) or mutant KRAS dimerization in BRET competition assays (FIG. 9d).

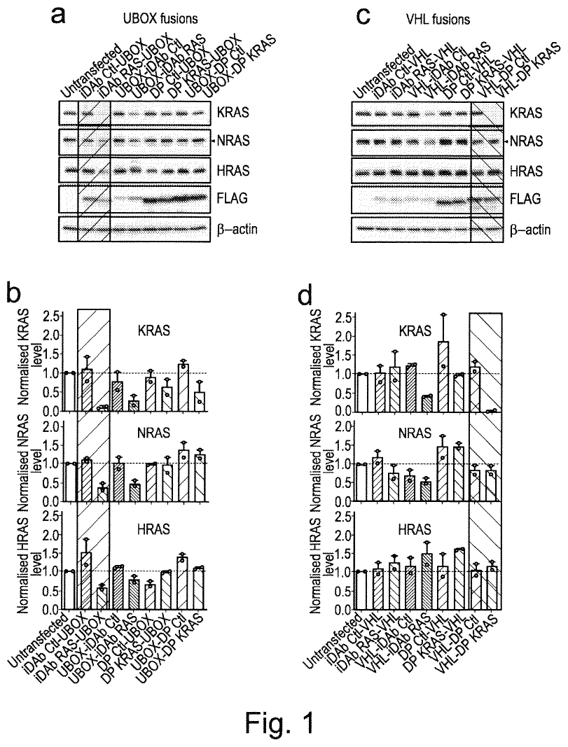

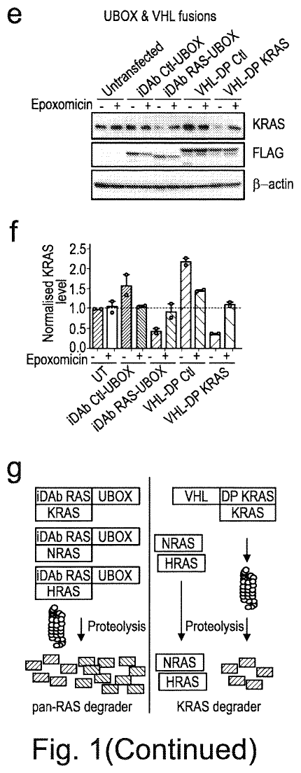

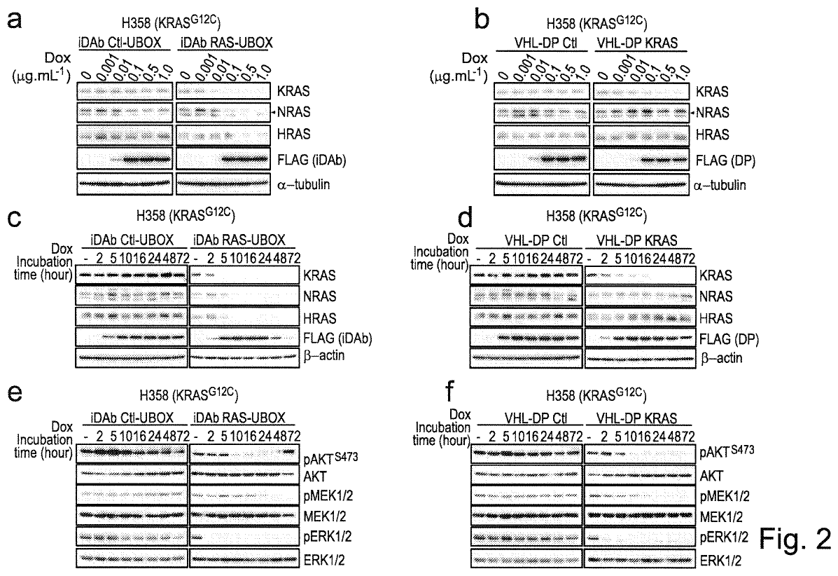

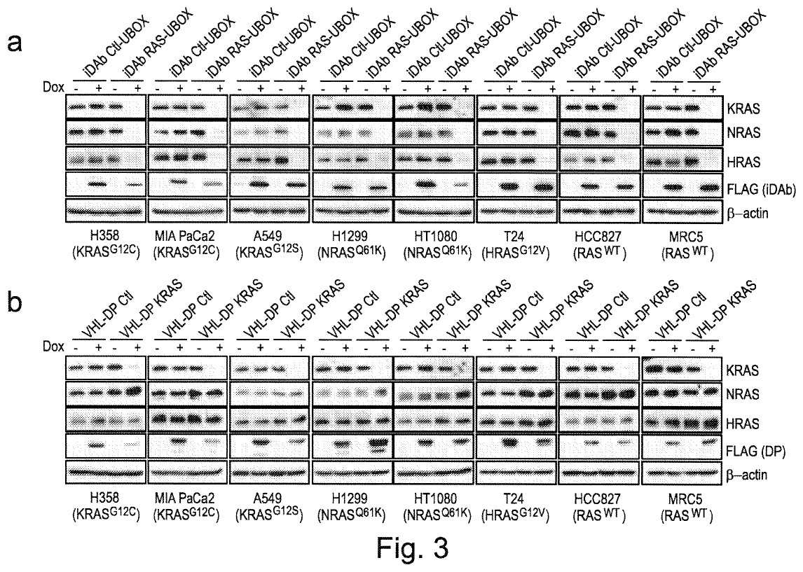

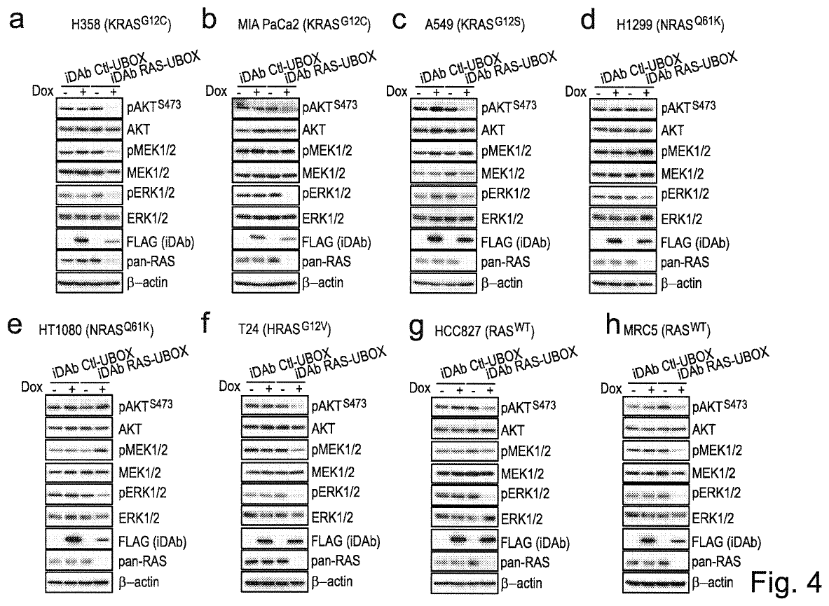

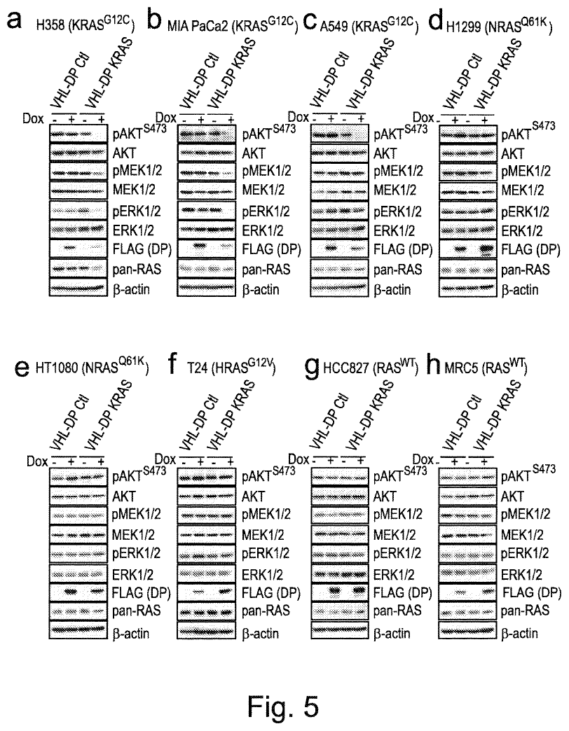

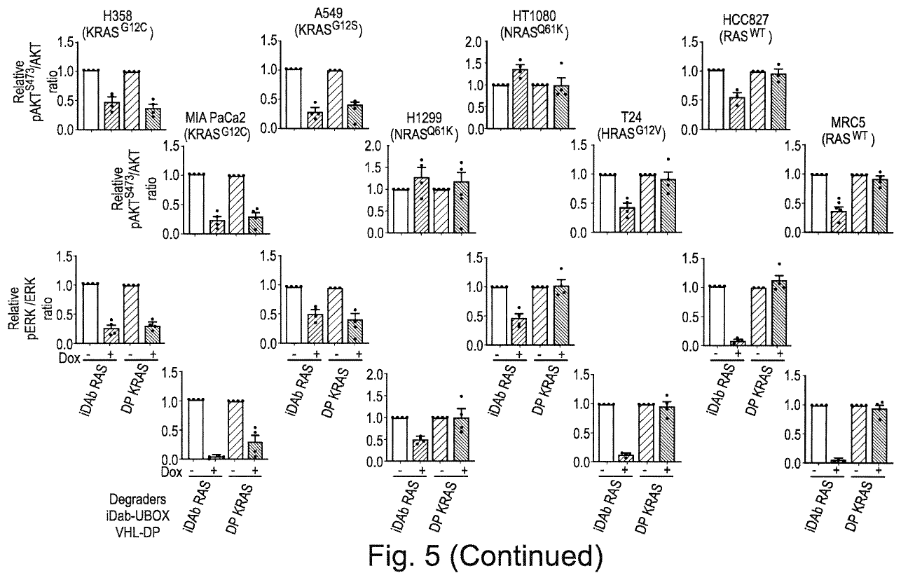

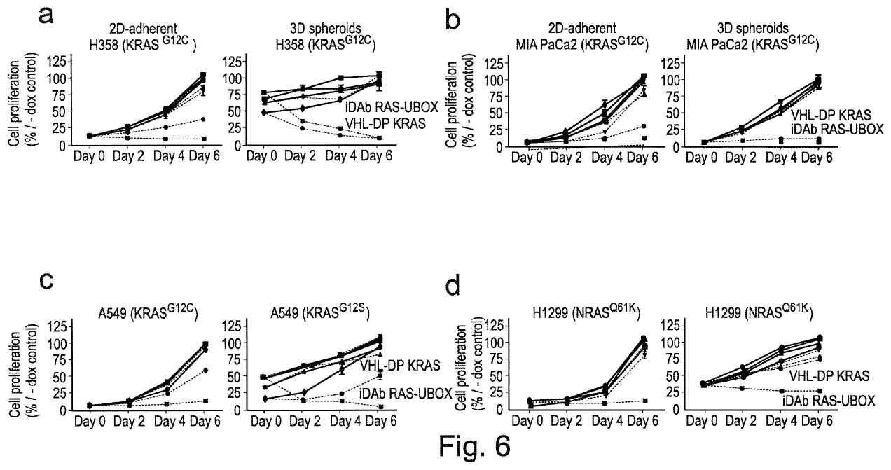

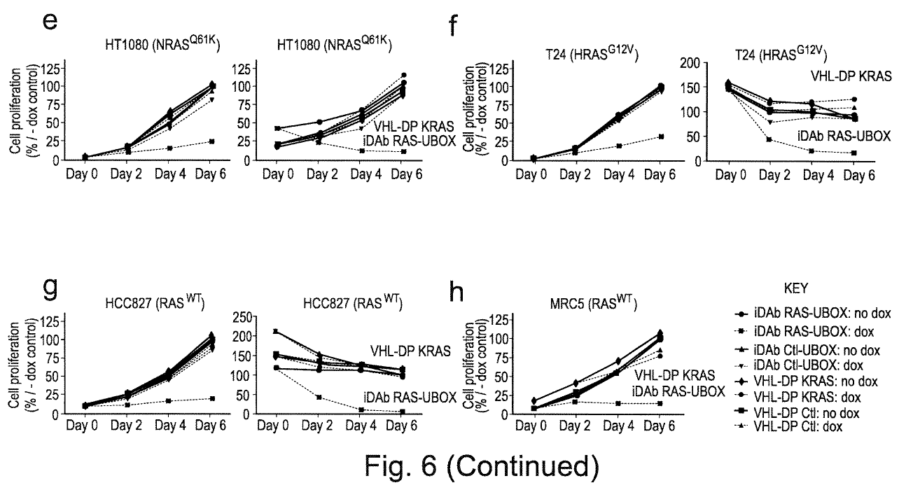

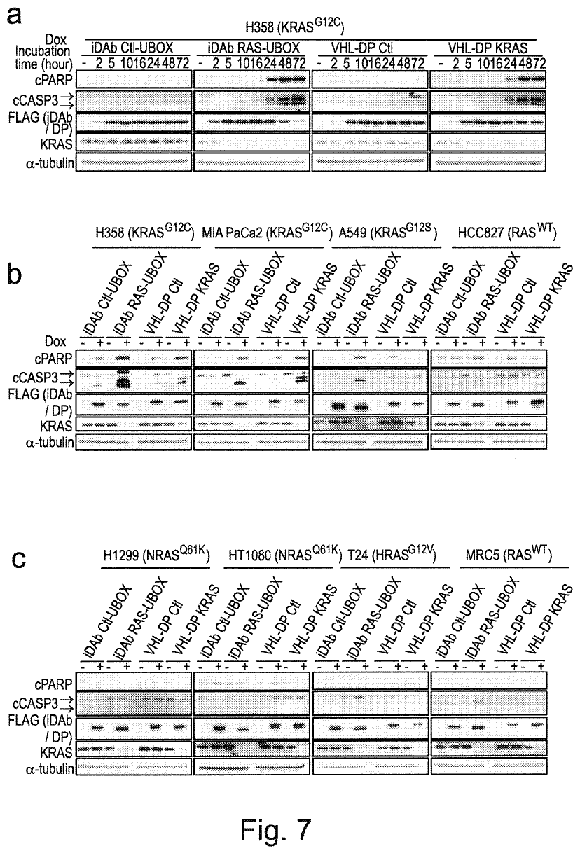

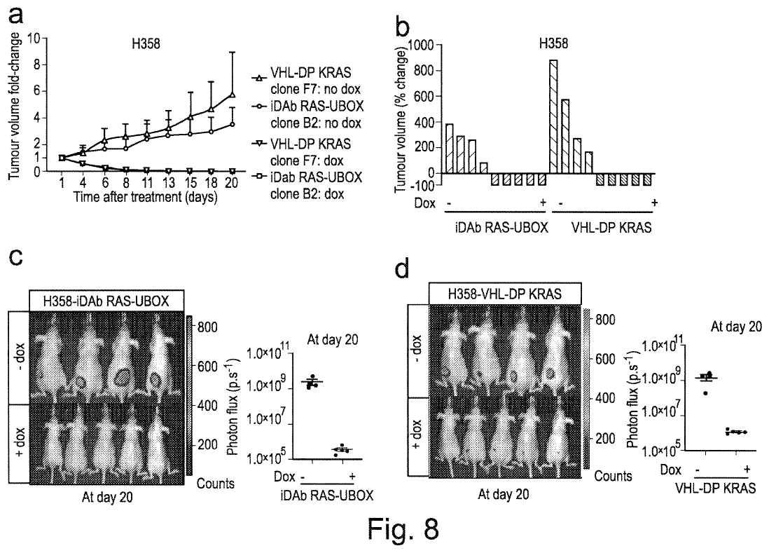

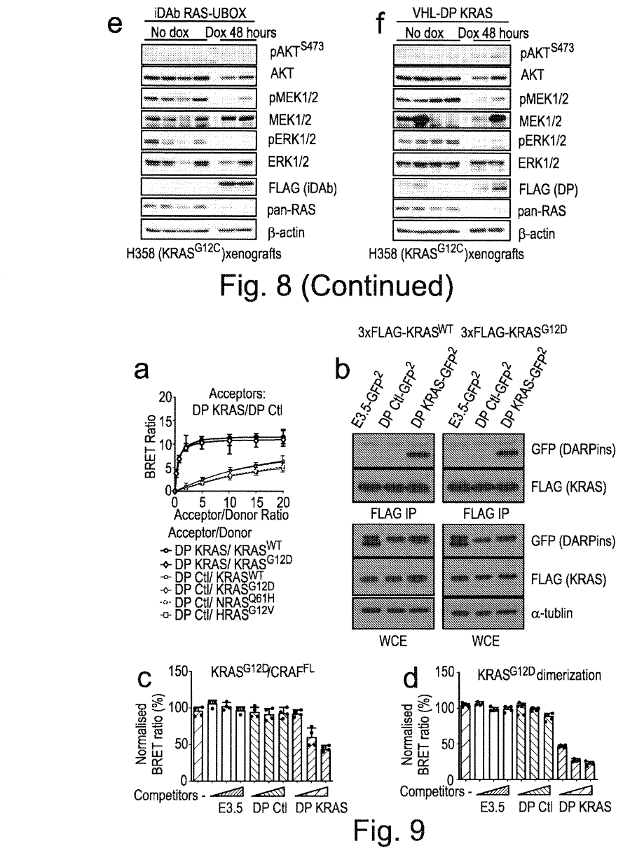

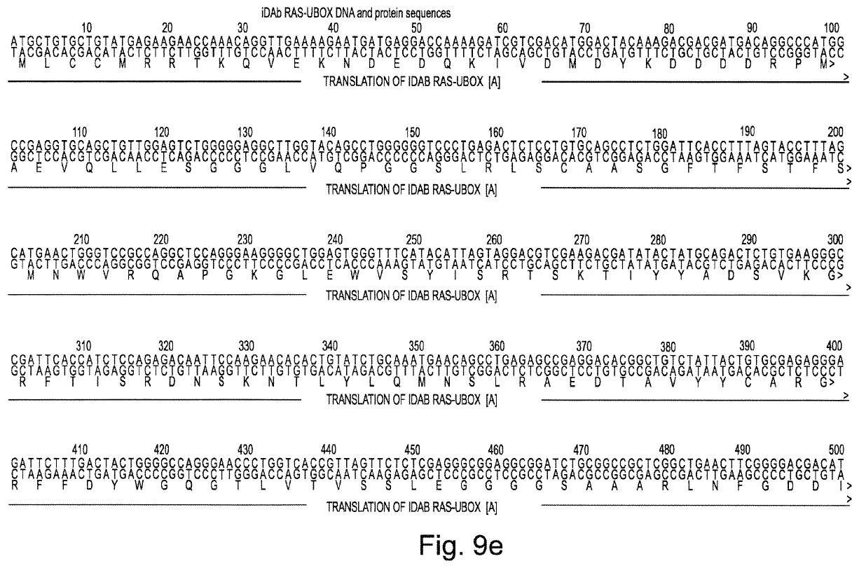

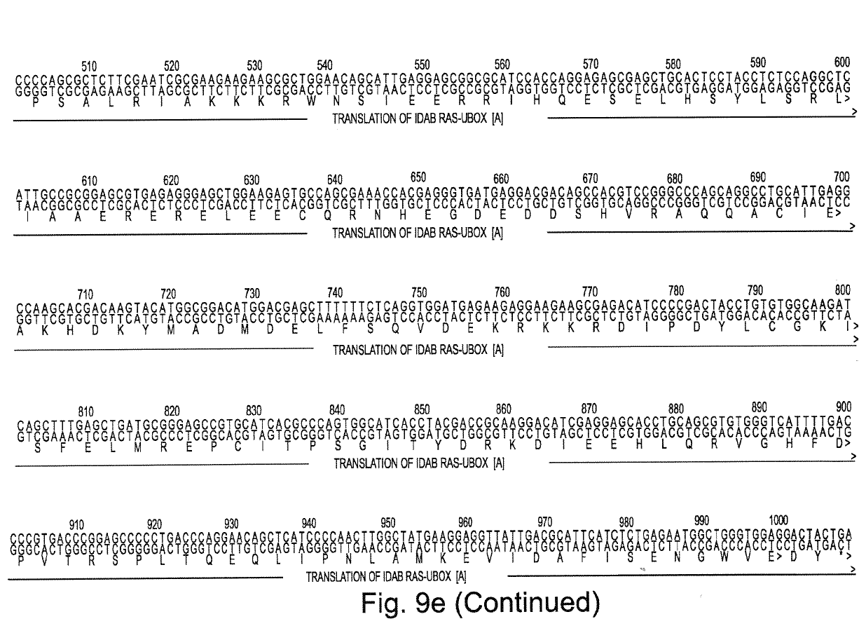

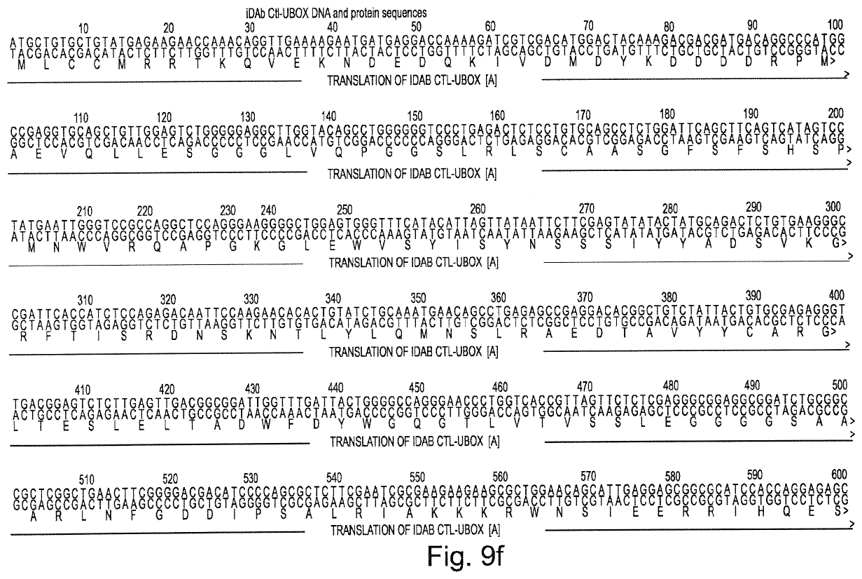

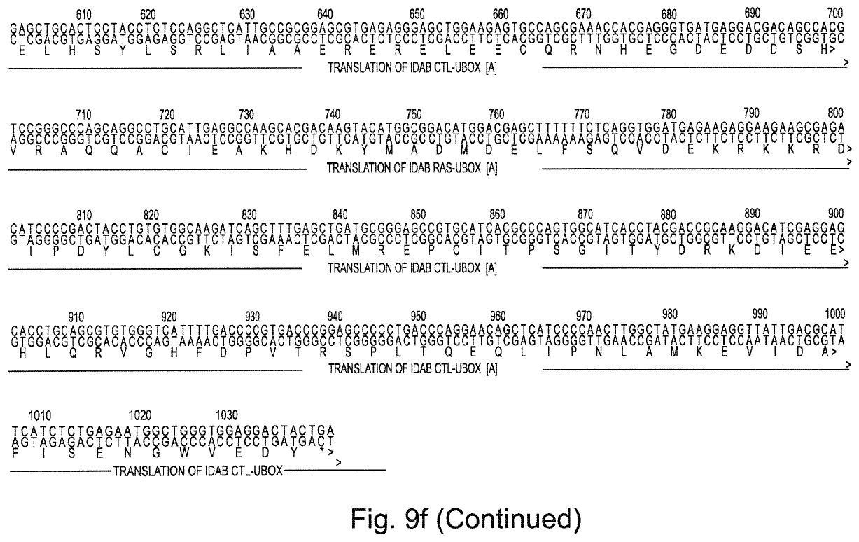

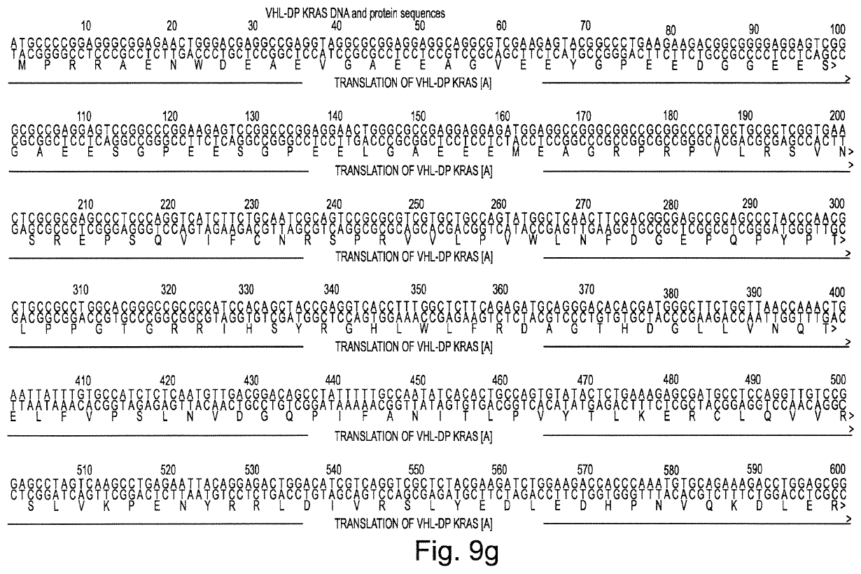

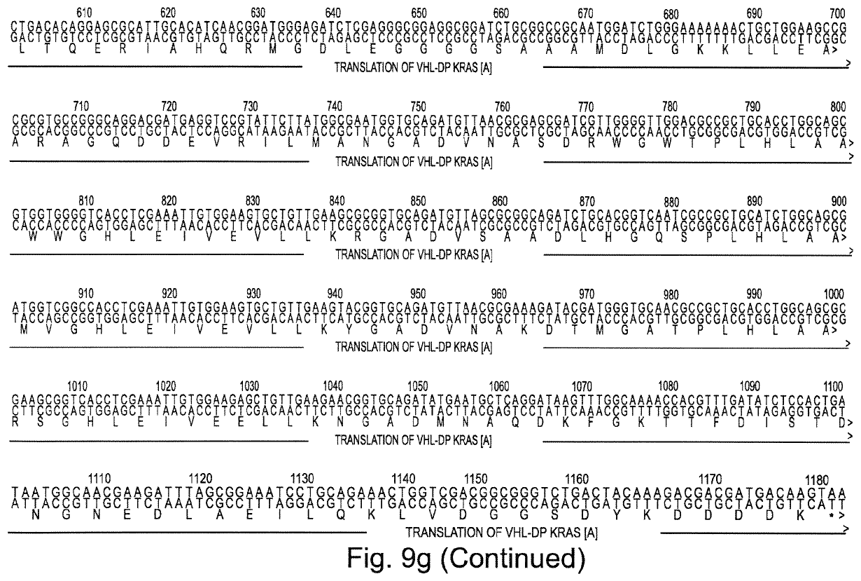

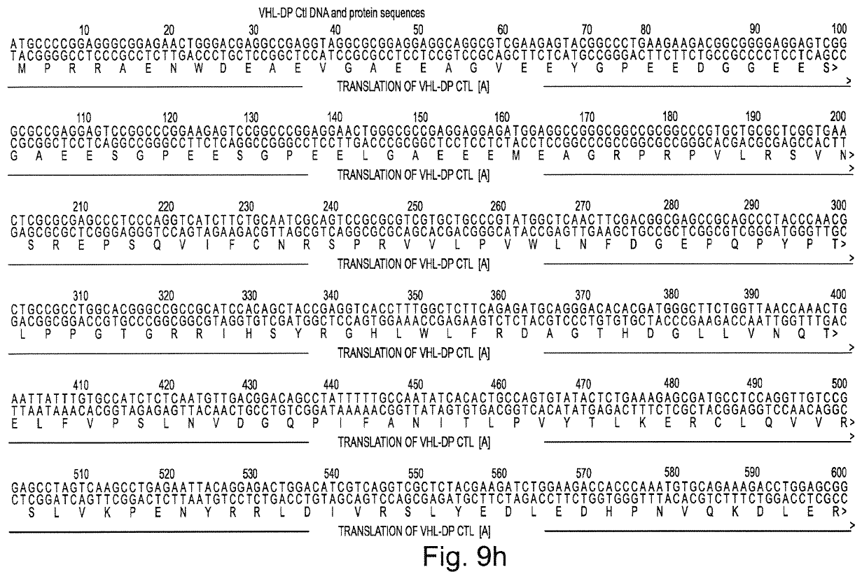

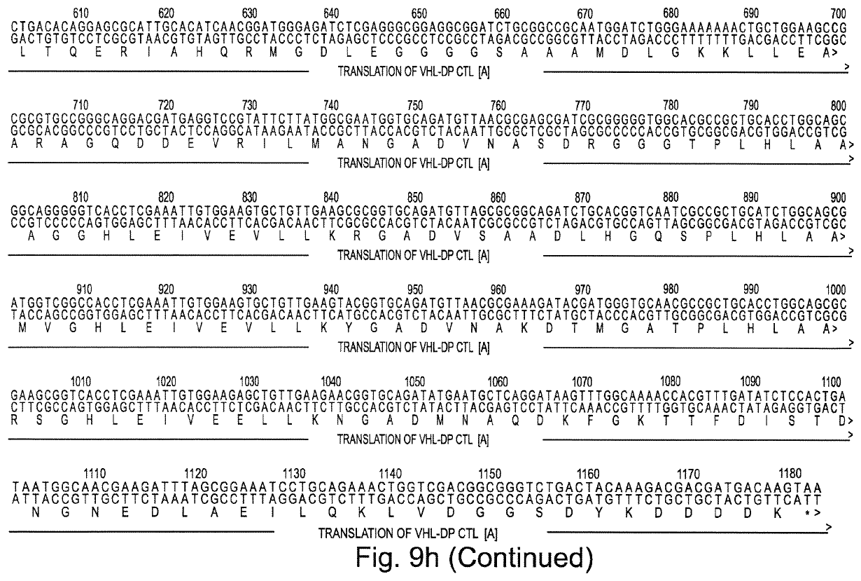

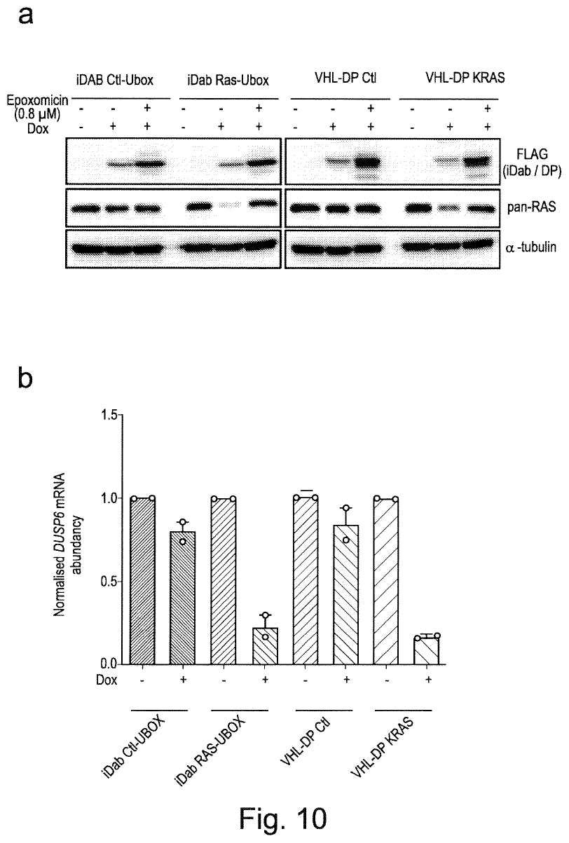

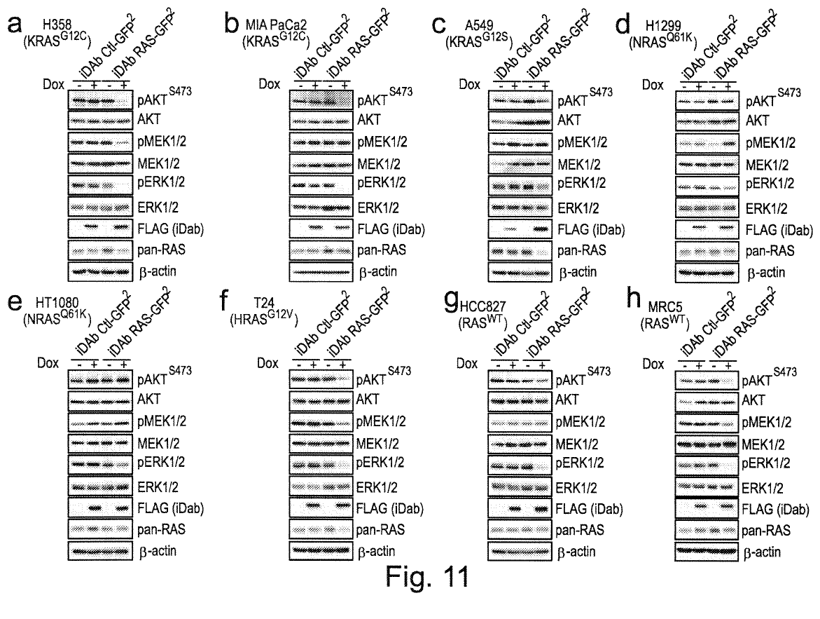

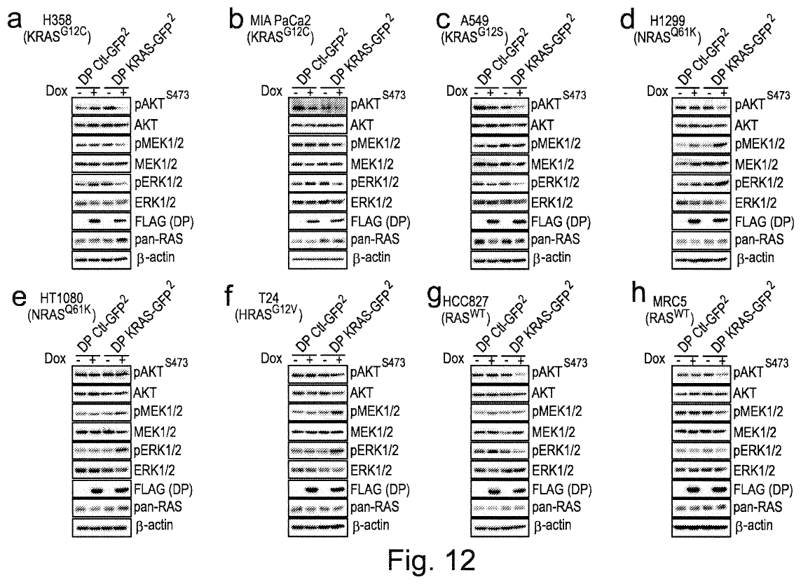

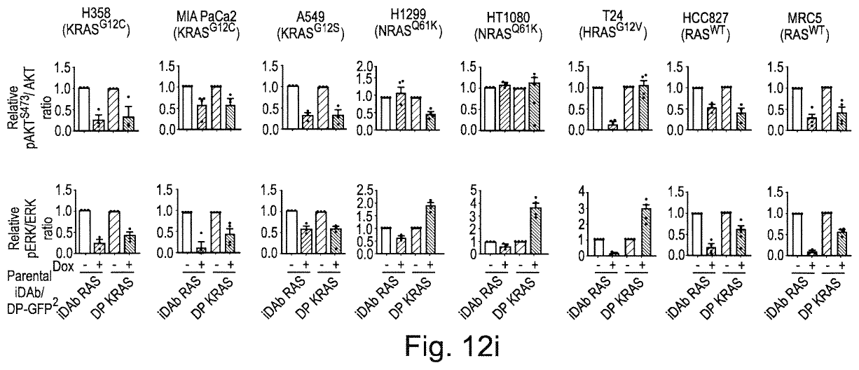

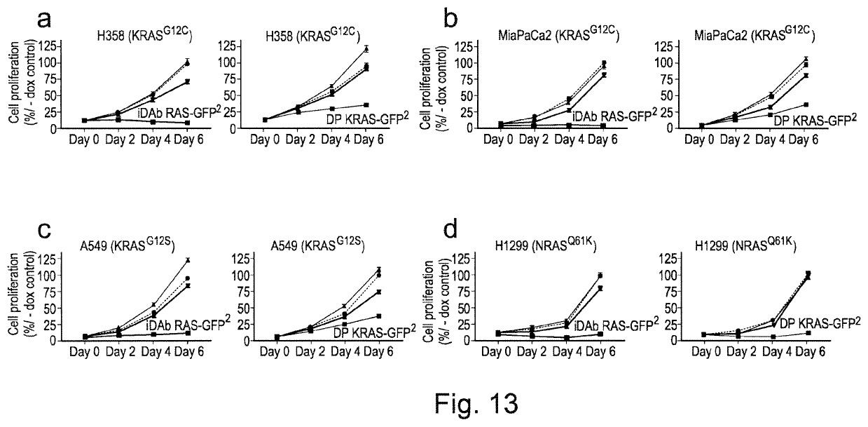

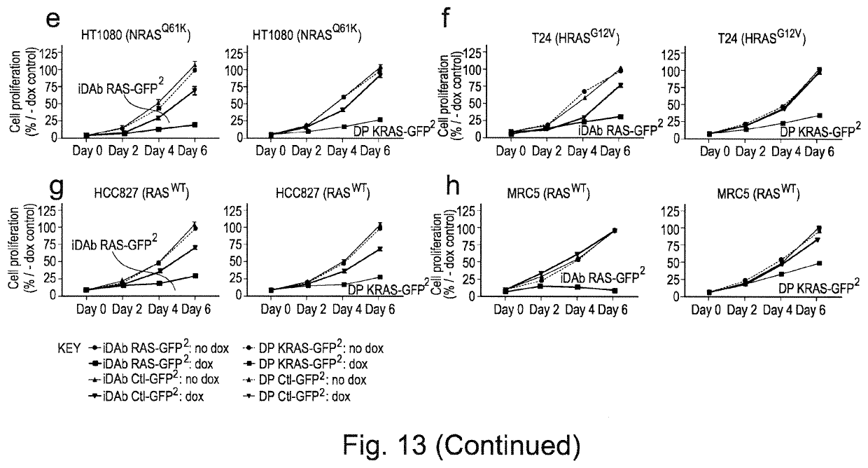

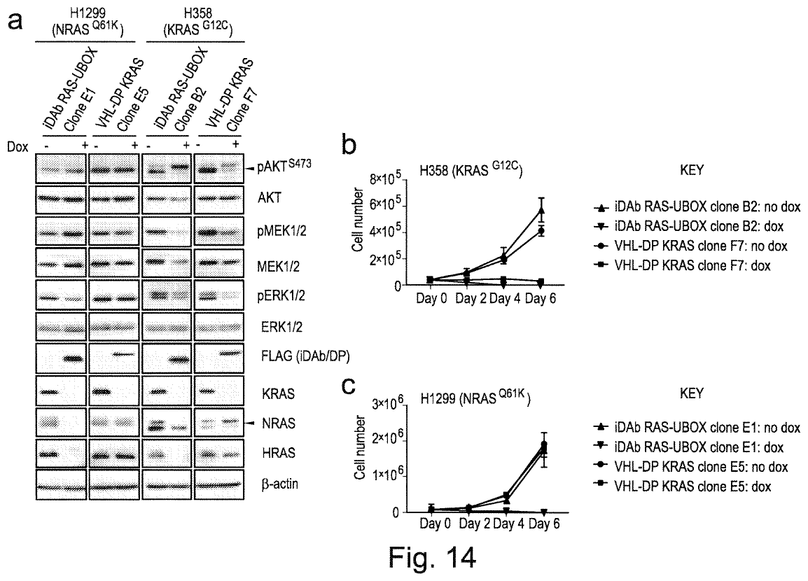

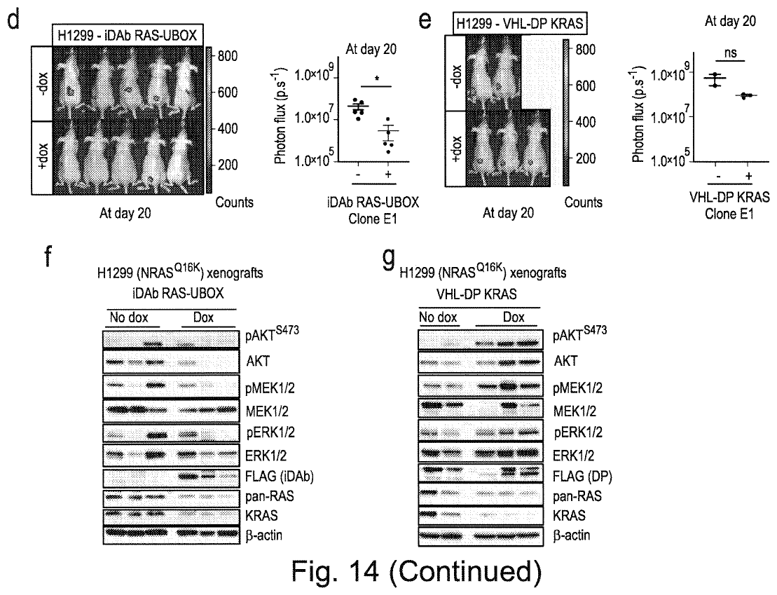

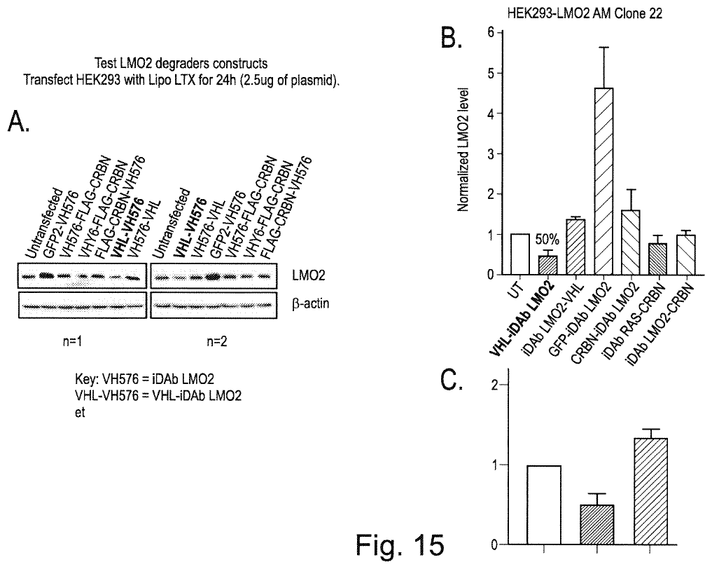

HCT116 cells (which express KRAS^{G13D}) were transiently transfected with these UBOX/single domain constructs and K/N/HRAS protein levels were monitored by Western blot. The iDAb RAS-UBOX and UBOX-iDAb RAS constructs both induced a decrease of KRAS, NRAS and HRAS protein levels, and the iDAb RAS-UBOX showing greater degradation (FIG. 1a, b). Conversely, we could detect little RAS turnover by the C-terminal VHL fusion with the iDAb RAS (i.e. iDAb RAS-VHL), although some degradation was observed with the N-terminal VHL fusion (i.e. VHL-iDAb RAS) (FIG. 1c, d). Similarly, UBOX fusions with the DP KRAS showed little effect on RAS protein levels (FIG. 1a, b). However, when we engineered a VHL fusion at the N-terminus of DP KRAS (VHL-DP KRAS), we observed a substantial decrease of KRAS protein in the transfected HCT116 cells (FIG. 1c, d). VHL-DP KRAS fusion diminished KRAS protein levels much more than DP KRAS-VHL fusion (FIG. 1d). A key observation is that, unlike iDAb RAS-UBOX, VHL-DP KRAS only depleted KRAS and not NRAS or HRAS (FIG. 1c, d) due to the KRAS-specific binding property of this DARPin. These degradation effects were proteasome dependent as epoxomicin treatment (a proteasome inhibitor) impeded RAS degradation for the iDAb-UBOX and the VHL-DP KRAS (FIG. 1e, f). Therefore, the functionalisation of iDAb RAS with UBOX domain (referred to here as “pan-RAS degrader”) and DP KRAS with VHL (referred to here as “KRAS degrader”) promotes RAS and KRAS degradation respectively (FIG. 1g, the sequences in Figure S1e-h).

### 3.2 KRAS Degrader Specifically Depletes KRAS in Human Cancer Cell Lines

We performed a comparison of the KRAS-specificity of the KRAS degrader with the pan-RAS degrader in a stably-transduced H358 (lung, KRAS^{G12C}) cancer cell line. The engineered proteins were expressed using a Tet-On inducible system (i.e. degrader expression is induced after doxycycline treatment) in cells transduced with lentiviral vectors. We first characterised the degrader properties in these H358 cells with an increasing dose of doxycycline induction, demonstrating a depletion of only KRAS with the KRAS degrader while the pan-RAS degrader knocked down of all three K/N/HRAS proteins, in a dose dependent manner (FIG. 2a, b). No effect was observed with the control degraders (FIG. 2a, b). These results confirmed the specificity of degradation observed in the transient transfection experiments shown in FIG. 1. We analysed the kinetics of RAS protein degradation following doxycycline induction of either the pan-RAS degrader or the KRAS degrader and the resultant effects of RAS-dependent downstream signalling. The synthesis of degrader proteins (detected using Western blot with anti-FLAG antibody) could be observed in as little as 2 hours after doxycycline treatment (FIG. 2c, d) and the loss of K, N and HRAS followed with similar profiles from 2 hours when the pan-RAS degrader was expressed (FIG. 2c). However, only the KRAS level was reduced when the KRAS degrader was expressed and the KRAS reduction was concomitant with the degrader expression (FIG. 2d). As expected, we also observed loss of phosphorylation of AKT, MEK and ERK in parallel with either degrader synthesis (FIG. 2e, f). RAS degradation was confirmed to be proteasome-dependent by treating cells with the proteasome inhibitor epoxomicin (Figure S2a). In addition, consistent with the potent inhibition of MAPK pathway activity, both pan-RAS and KRAS degraders reduced the expression of the MAPK pathway downstream transcript DUSP6 (Figure S2b). Our data demonstrate the KRAS-specific degrader causes rapid depletion of KRAS coupled to an inhibition of RAS downstream signalling and sustained degradation of KRAS over the doxycycline treatment period (as long as 72 hours). The pan-RAS degrader shows similar properties.

We also assessed the specificity of degradation of both degraders in several stably-transduced cancer cell lines in addition to H358, namely MIA PaCa2 (pancreas, KRAS^{G12C}) A549 (lung, KRAS^{G12S}), H1299 (lung, NRAS^{Q61K}), HT1080 (fibrosarcoma, NRAS^{Q61K}), T24 (bladder, HRAS^{G12V}), HCC827 (lung, RAS^{WT} but EGFR mutated) and an untransformed cell line: MRC5 (non-transformed lung fibroblast, RAS). The engineered proteins were again expressed using a Tet-On inducible system in cells transduced with lentiviral vectors. The degrader expression was detected in Western blots using anti-FLAG tag antibody on induction by doxycycline (FIG. 3) causing efficient knockdown of K, N and HRAS in cells expressing the pan-RAS (FIG. 3a) or degradation of KRAS only when the KRAS degrader was expressed (FIG. 3b). This occurred in all the cell lines tested.

### 3.3 KRAS Degrader Specifically Inhibits RAS Dependent Signalling of Mutant KRAS Cell Lines

The kinetics of RAS degradation in H358 cells was examined to determine the initiation and duration of effects on RAS signalling (FIG. 2). We further evaluated the consequences of pan-RAS or KRAS-only degradation on RAS downstream signalling pathways of the panel of cell lines using Western blots 72 hours after induction with doxycycline. The pan-RAS degrader induced loss of RAS proteins which resulted in the inhibition of RAS signalling (either PI3K and/or MAPK pathways). This was determined by reduction in phosphorylation of AKT, MEK and/or ERK, in all the cell lines, although not all proteins in the MAPK and PI3K pathways were similarly affected (FIGS. 4 & 5i). Loss of RAS signalling was also observed in two lines without RAS gene mutations (FIG. 4g, h) whereas the control iDAb Ctl-UBOX had no effect (FIG. 4a-h). Conversely, the KRAS degrader only inhibited AKT, ERK and MEK phosphorylation in those cells with mutant KRAS, namely H358, MIA PaCa2 and to a lesser extent in A549 (FIG. 5a-c, i). Indeed, it had no consequence on cells with mutation of NRAS (H1299 and HT1080) or HRAS (T24) or without mutant RAS (HCC827 and MRC5) (FIG. 5d-h). The VHL-DP Ctl had no effect (FIG. 5). These data are quantified in FIG. 5i. Expression of the pan-RAS iDAb fused to GFP² resulted in reduced RAS levels in H358, A549 and T24 (FIG. 11a-h) while the KRAS-specific DARPin fused to GFP² had no effect on the levels of RAS proteins (FIG. 12a-h). The iDAb RAS-GFP² had a similar inhibitory output on RAS downstream pathways than its degrader version (FIGS. 11a-h & 12i for quantification). The KRAS-specific DARPin fused to GFP² also decreased RAS signalling in mutant KRAS cells (H358, MIA PaCa2 and A549 in FIG. 12a-c, i), whereas it altered RAS signalling differently in the other cell lines (Figure S4d-i). Indeed, it augmented MEK and ERK phosphorylation, while it decreased or had no effect on pAKT levels in H1299, HT1080 and T24 cells, which have mutant NRAS or HRAS (FIG. 12d-f, i) and diminished pERK and pAKT in HCC827 and MRC5 cell lines lacking RAS mutation (FIG. 12g-i). The KRAS-specific DARPin interacts with KRAS-GTP and KRAS-GDP and may be a GAP inhibitor, therefore, the increase of pMEK/pERK signals might be attributed to its GAP inhibitory mechanism on KRAS^{WT}.

### 3.4 KRAS Degrader Specifically Inhibits the Proliferation of Mutant KRAS Cell Lines

Our data showed that the KRAS-specific DARPin K19 engineered into a degrader has focused specificity towards mutant KRAS cells compared to the parental DARPin. We assessed the effect of KRAS or pan-RAS degradation on the proliferation of our panel of cell lines with inducible macrodrugs by testing the proliferation in 2D-adherent and 3D spheroid assays. The pan-RAS degrader inhibited proliferation of all the cell lines, in both 2D-adherent or 3D spheroid assays (FIG. 6a-h) including MRC5 which is non-transformed and has wild type RAS. This was also observed with the parental iDAb RAS-GFP² in 2D-adherent proliferation assays (Figure S5a-h). KRAS degrader specifically reduced proliferation of cells with mutant KRAS expression in 2D-adherent and 3D spheroid assays (FIG. 6a-c) but did not modify the proliferation of cancer cell lines with mutant NRAS or HRAS (FIG. 6d-f) nor of the RAS^{WT} cells HCC827 and MRC5 (FIG. 6g, h). These data concur with the effect of KRAS degrader described on RAS signalling pathways in FIG. 5. On the contrary, the parental DARPin KRAS-GFP², like the pan-RAS iDAb-GFP² and the pan-RAS degrader, decreased the 2D-adherent proliferation of all the stable cell lines (FIG. 6 and Figure S5a-h), endorsing the benefit of engineering the DARPin KRAS into a KRAS-specific degrader.

The pan-RAS and the KRAS degraders both inhibited proliferation in H358 cells by inducing programmed cell death indicated by evidence of the apoptosis markers of cleaved PARP and cleaved caspase 3, starting from 16-24 hours after doxycycline addition, with the highest response at 72 hours (FIG. 7a). Consequently, we evaluated the induction of apoptosis markers after 72 hours of degrader expression in all the stable cell lines. The KRAS degrader and pan-RAS induced cleavage of caspase 3 and PARP in H358 and MIA PaCa2 cell lines but also in A549 cells upon expression of the pan-RAS degrader only (FIG. 7b). The other cell lines showed little cleaved PARP or caspase 3 compared to the negative controls (FIG. 7b, c). These results suggest that KRAS degrader blocked proliferation of cells expressing mutant KRAS by inducing apoptosis in KRAS dependent cells (i.e. H358 and MIA PaCa2), while it did not in KRAS independent cells (A549) in 2D-adherent culture method.

### 3.5 KRAS Degrader Induces Regression of Mutant KRAS Tumours In Vivo

We finally determined whether the degraders could be efficient in subcutaneous xenograft mouse models. From our previously established H358 and H1299 stable cell lines, we isolated unique clones of pan-RAS/KRAS degraders that would additionally express a Firefly Luciferase (FLuc) to detect the tumour in vivo. These individual clones were characterised in vitro by Western blot and growth curves. Induction of the expression of pan-RAS and KRAS degraders in H358 inhibited RAS downstream signalling pathways (FIG. 14a) and strongly impeded the cell growth (FIG. 14b). Only the pan-RAS degrader had an inhibitory effect on the RAS signalling pathways and the cell growth of H1299 cells (FIG. 14a, c) while the KRAS degrader had no effect, on either, in H1299 (FIG. 14a, c).

The growth of doxycycline-inducible cells in vivo was examined by subcutaneously injecting cells in nude mice to establish xenograft models. While the pan-RAS degrader significantly impeded H1299 tumours burden (FIG. 14d), the KRAS degrader caused almost no inhibition (FIG. 14e). However, expression of both pan-RAS and KRAS degraders in mutant KRAS H358 xenografts induced regression of tumours after only three days of doxycycline treatment with a substantial regression of all tumours after 20 days of treatment (FIG. 8a-d). In order to analyse RAS downstream signalling in H358 xenografts, two mice were treated with doxycycline for 48 hours before Western blot analysis of their tumour compared to the non-treated tumours because the doxycycline treated tumours regressed and could not be resected after 20 days of treatment. After 48 hours of doxycycline, we observed a large decrease in phosphorylation of MEK and ERK in H358 pan-RAS and KRAS degraders in parallel with degradation of their RAS target(s) (FIG. 8e, f. In contrast, in H1299 tumours, 20 days after treatment, we detected a decrease of phosphorylation of MEK and ERK kinases in tumours expressing the pan-RAS degrader (FIG. 14f and not the KRAS degrader (FIG. 14g).

In conclusion, our in vitro and in vivo data show that even though KRAS degrader depletes both endogenous KRAS^{WT} and KRAS^{MUT}, it only inhibits cancer cells expressing mutant KRAS.

### 4 Discussion

Effective cancer therapy based on developing reagents to intracellular targets that comprise families of proteins should ideally incorporate specific targeting of individual family members.

The RAS family is an important example in which three isoforms exist, each of which can undergo mutation in various tumour types. KRAS protein is the most often mutated isoform in human cancers, principally resulting from base changes causing single amino acid changes that are spread throughout the protein, but in mutational hotspots. Therefore, targeting mutant KRAS is challenging due to the number of different mutations and the high sequence identity between the three RAS isoforms (more than 80%). However, our recently described KRAS-specific DARPins showed the feasibility to specifically target both wild type and mutant KRAS by binding on the allosteric lobe of RAS. The addition of warheads to intracellular antibodies such as fusing procaspase to induce apoptosis or FBOX proteins to cause proteolysis provides a mechanism by which macromolecules could be converted to potent macrodrugs. While PROTAC small molecules have been described and are KRAS^{G12C} specific due to covalent interaction of compound to the protein, they did not degrade endogenous KRAS^{G12C} In addition, an affinity-directed protein missile system has shown degradation of KRAS and HRAS, but so far no isoform specific RAS degraders have been found.

In this study, we demonstrate a way forward to generalised KRAS inhibitors by engineering a KRAS-specific DARPin as a fusion protein with an E3 ligase to invoke proteasome targeting of KRAS and subsequent degradation. We have engineered our KRAS-specific DARPin with an E3 ligase warhead and compared this to an engineered pan-RAS binding iDAb. We found that RAS degradation could readily be achieved with both macrodrugs but that the specific E3 ligase and the terminal location of the E3 ligase on the macrodrug was important. Interestingly, the VHL E3 ligase was most efficient with the KRAS binding DARPin and the UBOX domain from CHIP E3 ligase with the pan-RAS iDAb. Both degraders allowed the efficient depletion of endogenous RAS proteins in multiple cell lines (i.e. from lung, pancreas, bladder, connective tissue) suggesting a broad applicability of this strategy. Furthermore, we showed the high specificity of the KRAS degrader that only depletes KRAS without affecting HRAS or NRAS protein level in all the cell lines tested. As previously described, we also observed that the degrader technology can modify the potency and/or the selectivity of the parental binder as shown here with the DP KRAS. In addition, the KRAS degrader only inhibited mutant KRAS cancer cells while the pan-RAS degrader showed no specificity for any RAS isoform mutant protein.

An important finding of our study is that KRAS^{WT} depletion by KRAS degrader did not lead to an inhibition of the proliferation of RAS' cells, especially untransformed cells. These data are supported by a previous study showing that expression of only one RAS in RASless mouse embryonic fibroblast did not impede their ability to proliferate while only the removal of the three RAS isoforms stopped the growth of these engineered fibroblast cells. This conclusion was confirmed in multiple cell lines with the pan-RAS degrader described here, strongly suggesting that mechanisms of compensation exist between the three RAS isoforms in RAS^{WT} cells in a way that loss of expression of one (or two) of the isoforms can be overcome via the expression of the other isoform(s). In addition, our data highlighted the efficacy of our KRAS degrader in vivo, with a rapid regression of mutant KRAS tumours. Therefore, KRAS-targeted degradation is an attractive therapeutic strategy for cancers with KRAS mutations, and not limited to any specific codon change.

Our study shows a proof of concept for the development of pan-KRAS specific degraders as therapeutics that could be implemented in any cancer with KRAS mutations. In the future, the design of combination therapies could be achieved to enhance the impact of KRAS degraders on these tumours, as previously done with KRAS^{G12C} inhibitors.

### Example 2—Chimaeric Proteins in Accordance with the Second Aspect of the Invention

LMO2 encodes a 18 kDa polypeptide that comprises two zinc-binding LIM domains. These domains are the interface for binding to class II basic helix-loop-helix (bHLH) transcription factors, such as TAL1/E2A and GATA. Furthermore, these two DNA-binding complexes are bridged by a scaffolding protein, LIM domain binding 1 (LDB1) that binds LMO2 on a different interface. This complex regulates the expression of genes important for the development and maintenance of T-ALL. LMO2 is overexpressed in more than 50% of T-ALL but also in subset of breast and prostate tumours but also in some diffuse large B cell lymphoma. General reviews of the role of LMO2 in normal and cancer cells will be known to those skilled in the art.

HEK293 cells stably expressing LMO2 (clones 18 and 22) were transfected with the indicated plasmid DNA constructs with Lipofectamine LTX. 2.5 μg of DNA were transfected per well of a 6-well plate for 24 hours. Cells were washed once with PBS and lysed in SDS-Tris buffer (STB: 1% SDS, 10 mM Tris-HCl pH 7.4) supplemented with protease inhibitors (Sigma) and phosphatase inhibitors (Thermo-Fisher). Cell lysates were sonicated with a Branson Sonifier.

The protein concentrations were determined by using the Pierce BCA protein assay kit (Thermo-Fisher). Equal amounts of protein (20 g) were resolved on 12.5% SDS-PAGE and subsequently transferred onto a PVDF membrane (GE Healthcare). The membrane was blocked with 10% non-fat milk (Sigma, Cat #70166) in TBS-0.1% Tween20 and incubated overnight with primary antibody at 4° C. (anti-FLAG (1/2000, Sigma, Cat #F3165), anti-LMO2 (1/1000, R&D, Cat #AF2726) and anti-β-actin (1/5000, Sigma, Cat #A1978)). After washing, the membrane was incubated with horse radish peroxidase-conjugated secondary antibody for 1 hour at 20° C. The membrane was washed with TBS-0.1% Tween and developed using Clarity Western ECL Substrate (Bio-Rad) and the ChemiDoc XRS+ imaging system (Bio-Rad).

The data show a substantial decrease of LMO2 protein level (50%) when VHL-VH576 is expressed in HEK293 cells stably expressing LMO2 compared to the untransfected condition and other E3 ligase-VH576 fusions. Note that GFP2-VH576 increases LMO2 protein level compared to the untransfected condition because it protects LMO2 from physiological degradation.

### SEQUENCE INFORMATION

| SEQ ID NO | Description of sequence |
|---|---|
| 1 | Amino acid sequence of human KRAS |
| 2 | Amino acid sequence of human HRAS |
| 3 | Amino acid sequence of human NRAS |
| 4 | Amino acid sequence of iDAb RAS |
| 5 | Amino acid sequence of UBOX-iDAb RAS |
| 6 | Amino acid sequence of DARPin K19 |
| 7 | DNA encoding DARPin K19 |
| 8 | Amino acid sequence of DARPin K13 |
| 9 | DNA encoding DARPin K13 |
| 10 | Amino acid sequence of P2-E2 |
| 11 | Amino acid sequence of P2-F3 |
| 12 | Amino acid sequence of human LMO2 |
| 13 | Amino acid sequence of VH576 |
| 14 | Amino acid sequence of VHL-VH576 |
| 15 | Amino acid sequence of Von Hippel-Lindau (VHL) E3 |
| | ligase |
| 16 | DNA encoding VHL E3 ligase |
| 17 | Amino acid sequence of UBOX |
| 18 | DNA encoding UBOX |
| 19 | Amino acid sequence of the linker sequence within |
| | VHL-DP-KRAS |
| 20 | Amino acid sequence of VHL-DP-KRAS |
| 21 | DNA sequence encoding VHL-DP-KRAS |
| 22 | Amino acid sequence of VHL-K13 |
| 23 | DNA sequence encoding VHL-K13 |
| 24 | Amino acid sequence of UBOX-DP KRAS |
| 25 | DNA sequence encoding UBOX-DP KRAS |
| 26 | Amino acid sequence of VHL-P2-E2 |
| 27 | Amino acid sequence of VHL-P2-F3 |
| 28 | Amino acid sequence of UBOX-P2-E2 |
| 29 | Amino acid sequence of UBOX-P2-F3 |
| 30 | Amino acid sequence of P2-E2-VHL |
| 31 | Amino acid sequence of P2-F3-VHL |
| 32 | Amino acid sequence of P2-E2-UBOX |
| 33 | Amino acid sequence of P2-F3-UBOX |
| 34 | Amino acid sequence of iDAb RAS-UBOX |
| 35 | Amino acid sequence of VHL-iDAb RAS |
| 36 | Primer |
| 37 | Primer |
| 38 | Primer |
| 39 | Primer |
| 40 | Optional sequence VDGGS |
| 41 | Optional FLAG tag sequence DYKDDDDK |

SEQ ID NO: 1
Amino acid sequence of WT human KRAS
MTEYKLVVVGAGGVGKSALTIQLIQNHFVDEYDPTIEDSYRKQVVIDGETCLLDILDTAGQEE
YSAMRDQYMRTGEGFLCVFAINNTKSFEDIHHYREQIKRVKDSEDVPMVLVGNKCDLPSRT
VDTKQAQDLARSYGIPFIETSAKTRQGVDDAFYTLVREIRKHKEKMSKDGKKKKKKSKTKCV
IM
SEQ ID NO: 2
Amino acid sequence of WT human HRAS
MTEYKLVVVGAGGVGKSALTIQLIQNHFVDEYDPTIEDSYRKQVVIDGETCLLDILDTAGQEE
YSAMRDQYMRTGEGFLCVFAINNTKSFEDIHQYREQIKRVKDSDDVPMVLVGNKCDLAART
VESRQAQDLARSYGIPYIETSAKTRQGVEDAFYTLVREIRQHKLRKLNPPDESGPGCMSCK
CVLS
SEQ ID NO: 3
Amino acid sequence of WT human NRAS
MTEYKLVVVGAGGVGKSALTIQLIQNHFVDEYDPTIEDSYRKQVVIDGETCLLDILDTAGQEE
YSAMRDQYMRTGEGFLCVFAINNSKSFADINLYREQIKRVKDSDDVPMVLVGNKCDLPTRT
VDTKQAHELAKSYGIPFIETSAKTRQGVEDAFYTLVREIRQYRMKKLNSSDDGTQGCMGLP
CVVM
SEQ ID NO: 4
Amino acid sequence of anti-pan RAS intracellular single domain antibody
iDAb RAS
MAEVQLLESGGGLVQPGGSLRLSCAASGFTFSTFSMNWVRQAPGKGLEWVSYISRTSKTI
YYADSVKGRFTISRDNSKNTLYLQMNSLRAEDTAVYYCARGRFFDYWGQGTLVTVSS
SEQ ID NO: 5
Amino acid sequence of UBOX-iDAb RAS
MRLNFGDDIPSALRIAKKKRWNSIEERRIHQESELHSYLSRLIAAERERELEECQRNHEGDE
DDSHVRAQQACIEAKHDKYMADMDELFSQVDEKRKKRDIPDYLCGKISFELMREPCITPSGI
TYDRKDIEEHLQRVGHFDPVTRSPLTQEQLIPNLAMKEVIDAFISENGWVEDYLEGGGGSA
AAMDYKDDDDKTSMAEVQLLESGGGLVQPGGSLRLSCAASGFTFSTFSMNWVRQAPGKG
LEWVSYISRTSKTIYYADSVKGRFTISRDNSKNTLYLQMNSLRAEDTAVYYCARGRFFDYW
GQGTLVTVSSKLNPPDESGPGCMSCKCVLS
SEQ ID NO: 6
Amino acid sequence of anti-KRAS DARPin K19 (“DP KRAS”)
MDLGKKLLEAARAGQDDEVRILMANGADVNASDRWGWTPLHLAAWWGHLEIVEVLLKRG
ADVSAADLHGQSPLHLAAMVGHLEIVEVLLKYGADVNAKDTMGATPLHLAARSGHLEIVEEL
LKNGADMNAQDKFGKTTFDISTDNGNEDLAEILQKL
SEQ ID NO: 7
DNA encoding DARPin K19
ATGGATCTGGGAAAAAAACTGCTGGAAGCCGCGCGTGCCGGGCAGGACGATGAGGTC
CGTATTCTTATGGCGAATGGTGCAGATGTTAACGCGAGCGATCGTTGGGGTTGGACGC
CGCTGCACCTGGCAGCGTGGTGGGGTCACCTCGAAATTGTGGAAGTGCTGTTGAAGC
GCGGTGCAGATGTTAGCGCGGCAGATCTGCACGGTCAATCGCCGCTGCATCTGGCAG
CGATGGTCGGCCACCTCGAAATTGTGGAAGTGCTGTTGAAGTACGGTGCAGATGTTAA
CGCGAAAGATACGATGGGTGCAACGCCGCTGCACCTGGCAGCGCGAAGCGGTCACCT
CGAAATTGTGGAAGAGCTGTTGAAGAACGGTGCAGATATGAATGCTCAGGATAAGTTTG
GCAAAACCACGTTTGATATCTCCACTGATAATGGCAACGAAGATTTAGCGGAAATCCTG
CAGAAACTG
SEQ ID NO: 8
Amino acid sequence of anti-KRAS DARPin K13
MDLGKKLLEAARAGQDDEVRILMANGADVNASDRWGWTPLHLAAWWGHLEIVEVLLKHG
ADVNAADLHGQTPLHLAAMVGHLEIVEVLLKYGADVNAKDTMGATPLHLAAQSGHLEIVEVL
LKNGADVNAQDKFGKTAFDISIDNGNEDLAEILQKL
SEQ ID NO: 9
DNA encoding K13
ATGGATCTGGGAAAAAAACTGCTGGAAGCCGCGCGTGCCGGACAGGACGATGAGGTC
CGTATTCTTATGGCGAACGGTGCAGATGTTAACGCGAGCGATCGCTGGGGTTGGACGC
CGCTGCATCTGGCAGCGTGGTGGGGTCACCTCGAAATTGTGGAAGTGCTGTTGAAGCA
CGGTGCAGATGTTAACGCGGCAGATCTGCACGGTCAAACGCCGCTGCATCTGGCAGC
GATGGTCGGTCACCTCGAAATTGTGGAAGTGCTGTTGAAGTACGGTGCAGATGTTAAC
GCGAAAGATACGATGGGTGCAACGCCGCTGCATCTGGCAGCGCAAAGCGGTCACCTC
GAAATTGTGGAAGTGCTGTTGAAGAACGGTGCAGATGTGAATGCTCAGGATAAGTTTG
GCAAAACCGCGTTTGATATCTCCATTGATAATGGCAACGAAGATTTAGCGGAAATCCTG
CAGAAACTG
SEQ ID NO: 10
Amino acid sequence of anti-KRAS scFv P2-E2
MAEVQLLESGGGSVQPGGSLRLSCAASGFTFSSYAMSWVRQAPGKGLEWVSAISGSGGS
TYYADSVKGRFTISRDNSKNTLYLQMNSLRAEDTAVYYCARDRDYGDYFDYWGQGTLVTV
SSLEGGGGGGGGSGGGASDIVMTQSPGTLSLSPGERATLSCRASQTVSTYLAWYQQKP
GQAPRLLIYGASSLATGIPDRFSGSGSGTDFTLTISRLEPEDAAVYYCQQYGSSVITFGQGT
KLEIKRAAASAHHHHHHKLDYKDHDGDYKDHDIDYKDDDDK
SEQ ID NO: 11
Amino acid sequence of anti-KRAS scFv P2-F3
MAEVQLVQSGAEVKKPGASVKVSCKASGYTFTGYYMHWVRQAPGQGLEWMGWINPNSG
GTNYAQKFQGRVTMTRDTSISTAYMELSRLRSDDTAVYYCARDRGSSWYVAFDYWGQGT
LVTVSSLEGGGGSGGGGSGGGASDIQMTQSPDSLAVSLGERATISCKSSQSVLYKYNNKN
YLAWYQQKPGQPPKLIIYWASTRESGVPDRFSGSGSGTDFTLTISSLQPEDVAVYYCQQYY
TTPYTFGQGTKVEIKRAAASAHHHHHHKLDYKDHDGDYKDHDIDYKDDDDK
SEQ ID NO: 12
Amino acid sequence of WT human LMO2
MSSAIERKSLDPSEEPVDEVLQIPPSLLTCGGCQQNIGDRYFLKAIDQYWHEDCLSCDLCG
CRLGEVGRRLYYKLGRKLCRRDYLRLFGQDGLCASCDKRIRAYEMTMRVKDKVYHLECFK
CAACQKHFCVGDRYLLINSDIVCEQDIYEWTKINGMI
SEQ ID NO: 13
Amino acid sequence of anti-LMO2 VH576
MAEVQLLESGGGLVQPGGSLRLSCAASGFSFSHSPMNWVRQAPGKGLEWVSYISYNSSSI
YYADSVKGRFTISRDNSKNTLYLQMNSLRAEDTAVYYCARGLTESLELTADWFDYWGQGT
LVTVSS
SEQ ID NO: 14
Amino acid sequence of VHL-VH576, an exemplary chimaeric protein of the
second aspect of the invention
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDLEGGGGSGGGGSGGGGSAARMAEVQLL
ESGGGLVQPGGSLRLSCAASGFSFSHSPMNWVRQAPGKGLEWVSYISYNSSSIYYADSVK
GRFTISRDNSKNTLYLQMNSLRAEDTAVYYCARGLTESLELTADWFDYWGQGTLVTVSSA
AAEQKLISEEDLNGAA
SEQ ID NO: 15
Amino acid sequence of VHL E3 domain
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGD
SEQ ID NO: 16
DNA encoding VHL E3 domain
ATGCCCCGGAGGGCGGAGAACTGGGACGAGGCCGAGGTAGGCGCGGAGGAGGCAG
GCGTCGAAGAGTACGGCCCTGAAGAAGACGGCGGGGAGGAGTCGGGCGCCGAGGAG
TCCGGCCCGGAAGAGTCCGGCCCGGAGGAACTGGGCGCCGAGGAGGAGATGGAGGC
CGGGCGGCCGCGGCCCGTGCTGCGCTCGGTGAACTCGCGCGAGCCCTCCCAGGTCA
TCTTCTGCAATCGCAGTCCGCGCGTCGTGCTGCCAGTATGGCTCAACTTCGACGGCGA
GCCGCAGCCCTACCCAACGCTGCCGCCTGGCACGGGCCGCCGCATCCACAGCTACCG
AGGTCACCTTTGGCTCTTCAGAGATGCAGGGACACACGATGGGCTTCTGGTTAACCAA
ACTGAATTATTTGTGCCATCTCTCAATGTTGACGGACAGCCTATTTTTGCCAATATCACA
CTGCCAGTGTATACTCTGAAAGAGCGATGCCTCCAGGTTGTCCGGAGCCTAGTCAAGC
CTGAGAATTACAGGAGACTGGACATCGTCAGGTCGCTCTACGAAGATCTGGAAGACCA
CCCAAATGTGCAGAAAGACCTGGAGCGGCTGACACAGGAGCGCATTGCACATCAACG
GATGGGAGAT
SEQ ID NO: 17
Amino acid sequence of UBOX domain of CHIP
MRLNFGDDIPSALRIAKKKRWNSIEERRIHQESELHSYLSRLIAAERERELEECQRNHEGDE
DDSHVRAQQACIEAKHDKYMADMDELFSQVDEKRKKRDIPDYLCGKISFELMREPCITPSGI
TYDRKDIEEHLQRVGHFDPVTRSPLTQEQLIPNLAMKEVIDAFISENGWVEDY
SEQ ID NO: 18
DNA sequence encoding UBOX domain of CHIP
ATGCGGCTGAACTTCGGGGACGACATCCCCAGCGCTCTTCGAATCGCGAAGAAGAAGC
GCTGGAACAGCATTGAGGAGCGGCGCATCCACCAGGAGAGCGAGCTGCACTCCTACC
TCTCCAGGCTCATTGCCGCGGAGCGTGAGAGGGAGCTGGAAGAGTGCCAGCGAAACC
ACGAGGGTGATGAGGACGACAGCCACGTCCGGGCCCAGCAGGCCTGCATTGAGGCCA
AGCACGACAAGTACATGGCGGACATGGACGAGCTTTTTTCTCAGGTGGATGAGAAGAG
GAAGAAGCGAGACATCCCCGACTACCTGTGTGGCAAGATCAGCTTTGAGCTGATGCGG
GAGCCGTGCATCACGCCCAGTGGCATCACCTACGACCGCAAGGACATCGAGGAGCAC
CTGCAGCGTGTGGGTCATTTTGACCCCGTGACCCGGAGCCCCCTGACCCAGGAACAG
CTCATCCCCAACTTGGCTATGAAGGAGGTTATTGACGCATTCATCTCTGAGAATGGCTG
GGTGGAGGACTAC
SEQ ID NO: 19
Amino acid sequence of exemplary linker
GGGGS
SEQ ID NO: 20
Amino acid sequence of VHL-DP KRAS (also referred to as VHL-K19), an
exemplary chimaeric protein of the first aspect of the invention
The KRAS-binding tryptophan repeats within the KRAS-specific endogenous
targeting portion are underlined.
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDLEGGGGSAAAMDLGKKLLEAARAGQDD
EVRILMANGADVNASDRWGWTPLHLAAWWGHLEIVEVLLKRGADVSAADLHGQSPLHLAA
MVGHLEIVEVLLKYGADVNAKDTMGATPLHLAARSGHLEIVEELLKNGADMNAQDKFGKTT
FDISTDNGNEDLAEILQKLVDGGSDYKDDDDK
SEQ ID NO: 21
DNA sequence encoding protein VHL-DP KRAS
ATGCCCCGGAGGGCGGAGAACTGGGACGAGGCCGAGGTAGGCGCGGAGGAGGCAG
GCGTCGAAGAGTACGGCCCTGAAGAAGACGGCGGGGAGGAGTCGGGCGCCGAGGAG
TCCGGCCCGGAAGAGTCCGGCCCGGAGGAACTGGGCGCCGAGGAGGAGATGGAGGC
CGGGCGGCCGCGGCCCGTGCTGCGCTCGGTGAACTCGCGCGAGCCCTCCCAGGTCA
TCTTCTGCAATCGCAGTCCGCGCGTCGTGCTGCCAGTATGGCTCAACTTCGACGGCGA
GCCGCAGCCCTACCCAACGCTGCCGCCTGGCACGGGCCGCCGCATCCACAGCTACCG
AGGTCACCTTTGGCTCTTCAGAGATGCAGGGACACACGATGGGCTTCTGGTTAACCAA
ACTGAATTATTTGTGCCATCTCTCAATGTTGACGGACAGCCTATTTTTGCCAATATCACA
CTGCCAGTGTATACTCTGAAAGAGCGATGCCTCCAGGTTGTCCGGAGCCTAGTCAAGC
CTGAGAATTACAGGAGACTGGACATCGTCAGGTCGCTCTACGAAGATCTGGAAGACCA
CCCAAATGTGCAGAAAGACCTGGAGCGGCTGACACAGGAGCGCATTGCACATCAACG
GATGGGAGATCTCGAGGGCGGAGGCGGATCTGCGGCCGCAATGGATCTGGGAAAAAA
ACTGCTGGAAGCCGCGCGTGCCGGGCAGGACGATGAGGTCCGTATTCTTATGGCGAA
TGGTGCAGATGTTAACGCGAGCGATCGTTGGGGTTGGACGCCGCTGCACCTGGCAGC
GTGGTGGGGTCACCTCGAAATTGTGGAAGTGCTGTTGAAGCGCGGTGCAGATGTTAGC
GCGGCAGATCTGCACGGTCAATCGCCGCTGCATCTGGCAGCGATGGTCGGCCACCTC
GAAATTGTGGAAGTGCTGTTGAAGTACGGTGCAGATGTTAACGCGAAAGATACGATGG
GTGCAACGCCGCTGCACCTGGCAGCGCGAAGCGGTCACCTCGAAATTGTGGAAGAGC
TGTTGAAGAACGGTGCAGATATGAATGCTCAGGATAAGTTTGGCAAAACCACGTTTGAT
ATCTCCACTGATAATGGCAACGAAGATTTAGCGGAAATCCTGCAGAAACTGGTCGACG
GCGGGTCTGACTACAAAGACGACGATGACAAGTAA
SEQ ID NO: 22
Amino acid sequence of VHL-K13, an exemplary chimaeric protein of the
first aspect of the invention
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDLEGGGGSAAAMDLGKKLLEAARAGQDD
EVRILMANGADVNASDRWGWTPLHLAAWWGHLEIVEVLLKHGADVNAADLHGQTPLHLAA
MVGHLEIVEVLLKYGADVNAKDTMGATPLHLAAQSGHLEIVEVLLKNGADVNAQDKFGKTA
FDISIDNGNEDLAEILQKLVDGGSDYKDDDDK*
SEQ ID NO: 23
DNA sequence encoding VHL-K13
ATGCCCCGGAGGGCGGAGAACTGGGACGAGGCCGAGGTAGGCGCGGAGGAGGCAG
GCGTCGAAGAGTACGGCCCTGAAGAAGACGGCGGGGAGGAGTCGGGCGCCGAGGAG
TCCGGCCCGGAAGAGTCCGGCCCGGAGGAACTGGGCGCCGAGGAGGAGATGGAGGC
CGGGCGGCCGCGGCCCGTGCTGCGCTCGGTGAACTCGCGCGAGCCCTCCCAGGTCA
TCTTCTGCAATCGCAGTCCGCGCGTCGTGCTGCCAGTATGGCTCAACTTCGACGGCGA
GCCGCAGCCCTACCCAACGCTGCCGCCTGGCACGGGCCGCCGCATCCACAGCTACCG
AGGTCACCTTTGGCTCTTCAGAGATGCAGGGACACACGATGGGCTTCTGGTTAACCAA
ACTGAATTATTTGTGCCATCTCTCAATGTTGACGGACAGCCTATTTTTGCCAATATCACA
CTGCCAGTGTATACTCTGAAAGAGCGATGCCTCCAGGTTGTCCGGAGCCTAGTCAAGC
CTGAGAATTACAGGAGACTGGACATCGTCAGGTCGCTCTACGAAGATCTGGAAGACCA
CCCAAATGTGCAGAAAGACCTGGAGCGGCTGACACAGGAGCGCATTGCACATCAACG
GATGGGAGATCTCGAGGGCGGAGGCGGATCTGCGGCCGCAATGGATCTGGGAAAAAA
ACTGCTGGAAGCCGCGCGTGCCGGACAGGACGATGAGGTCCGTATTCTTATGGCGAA
CGGTGCAGATGTTAACGCGAGCGATCGCTGGGGTTGGACGCCGCTGCATCTGGCAGC
GTGGTGGGGTCACCTCGAAATTGTGGAAGTGCTGTTGAAGCACGGTGCAGATGTTAAC
GCGGCAGATCTGCACGGTCAAACGCCGCTGCATCTGGCAGCGATGGTCGGTCACCTC
GAAATTGTGGAAGTGCTGTTGAAGTACGGTGCAGATGTTAACGCGAAAGATACGATGG
GTGCAACGCCGCTGCATCTGGCAGCGCAAAGCGGTCACCTCGAAATTGTGGAAGTGCT
GTTGAAGAACGGTGCAGATGTGAATGCTCAGGATAAGTTTGGCAAAACCGCGTTTGATA
TCTCCATTGATAATGGCAACGAAGATTTAGCGGAAATCCTGCAGAAACTGGTCGACGGC
GGGTCTGACTACAAAGACGACGATGACAAGTAA
SEQ ID NO: 24
Amino acid sequence of UBOX-DP-KRAS, an exemplary chimaeric protein of
the first aspect of the invention
MRLNFGDDIPSALRIAKKKRWNSIEERRIHQESELHSYLSRLIAAERERELEECQRNHEGDE
DDSHVRAQQACIEAKHDKYMADMDELFSQVDEKRKKRDIPDYLCGKISFELMREPCITPSGI
TYDRKDIEEHLQRVGHFDPVTRSPLTQEQLIPNLAMKEVIDAFISENGWVEDYLEGGGGSA
AAMDLGKKLLEAARAGQDDEVRILMANGADVNASDRWGWTPLHLAAWWGHLEIVEVLLKR
GADVSAADLHGQSPLHLAAMVGHLEIVEVLLKYGADVNAKDTMGATPLHLAARSGHLEIVE
ELLKNGADMNAQDKFGKTTFDISTDNGNEDLAEILQKLVDGGSDYKDDDDK
SEQ ID NO: 25
DNA sequence encoding UBOX-DP-KRAS
ATGCGGCTGAACTTCGGGGACGACATCCCCAGCGCTCTTCGAATCGCGAAGAAGAAGC
GCTGGAACAGCATTGAGGAGCGGCGCATCCACCAGGAGAGCGAGCTGCACTCCTACC
TCTCCAGGCTCATTGCCGCGGAGCGTGAGAGGGAGCTGGAAGAGTGCCAGCGAAACC
ACGAGGGTGATGAGGACGACAGCCACGTCCGGGCCCAGCAGGCCTGCATTGAGGCCA
AGCACGACAAGTACATGGCGGACATGGACGAGCTTTTTTCTCAGGTGGATGAGAAGAG
GAAGAAGCGAGACATCCCCGACTACCTGTGTGGCAAGATCAGCTTTGAGCTGATGCGG
GAGCCGTGCATCACGCCCAGTGGCATCACCTACGACCGCAAGGACATCGAGGAGCAC
CTGCAGCGTGTGGGTCATTTTGACCCCGTGACCCGGAGCCCCCTGACCCAGGAACAG
CTCATCCCCAACTTGGCTATGAAGGAGGTTATTGACGCATTCATCTCTGAGAATGGCTG
GGTGGAGGACTACCTCGAGGGCGGAGGCGGATCTGCGGCCGCAATGGATCTGGGAAA
AAAACTGCTGGAAGCCGCGCGTGCCGGGCAGGACGATGAGGTCCGTATTCTTATGGC
GAATGGTGCAGATGTTAACGCGAGCGATCGTTGGGGTTGGACGCCGCTGCACCTGGC
AGCGTGGTGGGGTCACCTCGAAATTGTGGAAGTGCTGTTGAAGCGCGGTGCAGATGTT
AGCGCGGCAGATCTGCACGGTCAATCGCCGCTGCATCTGGCAGCGATGGTCGGCCAC
CTCGAAATTGTGGAAGTGCTGTTGAAGTACGGTGCAGATGTTAACGCGAAAGATACGAT
GGGTGCAACGCCGCTGCACCTGGCAGCGCGAAGCGGTCACCTCGAAATTGTGGAAGA
GCTGTTGAAGAACGGTGCAGATATGAATGCTCAGGATAAGTTTGGCAAAACCACGTTTG
ATATCTCCACTGATAATGGCAACGAAGATTTAGCGGAAATCCTGCAGAAACTGGTCGAC
GGCGGGTCTGACTACAAAGACGACGATGACAAG
SEQ ID NO: 26
Amino acid sequence of VHL-P2-E2, an exemplary chimaeric protein of the
first aspect of the invention
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDGGGGGGGGSGGGGSGGGGSMAEVQL
LESGGGSVQPGGSLRLSCAASGFTFSSYAMSWVRQAPGKGLEWVSAISGSGGSTYYADS
VKGRFTISRDNSKNTLYLQMNSLRAEDTAVYYCARDRDYGDYFDYWGQGTLVTVSSLEGG
GGSGGGGSGGGASDIVMTQSPGTLSLSPGERATLSCRASQTVSTYLAWYQQKPGQAPRL
LIYGASSLATGIPDRFSGSGSGTDFTLTISRLEPEDAAVYYCQQYGSSVITFGQGTKLEIKRA
AASA
SEQ ID NO: 27
Amino acid sequence of VHL-P2-F3, an exemplary chimaeric protein of the
first aspect of the invention
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDGGGGSGGGGSGGGGSGGGGSMAEVQL
VQSGAEVKKPGASVKVSCKASGYTFTGYYMHWVRQAPGQGLEWMGWINPNSGGTNYAQ
KFQGRVTMTRDTSISTAYMELSRLRSDDTAVYYCARDRGSSWYVAFDYWGQGTLVTVSSL
EGGGGSGGGGSGGGASDIQMTQSPDSLAVSLGERATISCKSSQSVLYKYNNKNYLAWYQ
QKPGQPPKLIIYWASTRESGVPDRFSGSGSGTDFTLTISSLQPEDVAVYYCQQYYTTPYTF
GQGTKVEIKRAAASA
SEQ ID NO: 28
Amino acid sequence of UBOX-P2-E2, an exemplary chimaeric protein of the
first aspect of the invention
MRLNFGDDIPSALRIAKKKRWNSIEERRIHQESELHSYLSRLIAAERERELEECQRNHEGDE
DDSHVRAQQACIEAKHDKYMADMDELFSQVDEKRKKRDIPDYLCGKISFELMREPCITPSGI
TYDRKDIEEHLQRVGHFDPVTRSPLTQEQLIPNLAMKEVIDAFISENGWVEDYGGGGSGGG
GSGGGGSGGGGSMAEVQLLESGGGSVQPGGSLRLSCAASGFTFSSYAMSWVRQAPGK
GLEWVSAISGSGGSTYYADSVKGRFTISRDNSKNTLYLQMNSLRAEDTAVYYCARDRDYG
DYFDYWGQGTLVTVSSLEGGGGSGGGGSGGGASDIVMTQSPGTLSLSPGERATLSCRAS
QTVSTYLAWYQQKPGQAPRLLIYGASSLATGIPDRFSGSGSGTDFTLTISRLEPEDAAVYYC
QQYGSSVITFGQGTKLEIKRAAASA
SEQ ID NO: 29
Amino acid sequence of UBOX-P2-F3, an exemplary chimaeric protein of the
first aspect of the invention
MRLNFGDDIPSALRIAKKKRWNSIEERRIHQESELHSYLSRLIAAERERELEECQRNHEGDE
DDSHVRAQQACIEAKHDKYMADMDELFSQVDEKRKKRDIPDYLCGKISFELMREPCITPSGI
TYDRKDIEEHLQRVGHFDPVTRSPLTQEQLIPNLAMKEVIDAFISENGWVEDYGGGGSGGG
GSGGGGSGGGGSMAEVQLVQSGAEVKKPGASVKVSCKASGYTFTGYYMHWVRQAPGQ
GLEWMGWINPNSGGTNYAQKFQGRVTMTRDTSISTAYMELSRLRSDDTAVYYCARDRGS
SWYVAFDYWGQGTLVTVSSLEGGGGSGGGGSGGGASDIQMTQSPDSLAVSLGERATISC
KSSQSVLYKYNNKNYLAWYQQKPGQPPKLIIYWASTRESGVPDRFSGSGSGTDFTLTISSL
QPEDVAVYYCQQYYTTPYTFGQGTKVEIKRAAASA
SEQ ID NO: 30
Amino acid sequence of P2-E2-VHL, an exemplary chimaeric protein of the
first aspect of the invention
MAEVQLLESGGGSVQPGGSLRLSCAASGFTFSSYAMSWVRQAPGKGLEWVSAISGSGGS
TYYADSVKGRFTISRDNSKNTLYLQMNSLRAEDTAVYYCARDRDYGDYFDYWGQGTLVTV
SSLEGGGGSGGGGSGGGASDIVMTQSPGTLSLSPGERATLSCRASQTVSTYLAWYQQKP
GQAPRLLIYGASSLATGIPDRFSGSGSGTDFTLTISRLEPEDAAVYYCQQYGSSVITFGQGT
KLEIKRAAASAGGGGSGGGGSGGGGSGGGGSMPRRAENWDEAEVGAEEAGVEEYGPEE
DGGEESGAEESGPEESGPEELGAEEEMEAGRPRPVLRSVNSREPSQVIFCNRSPRVVLPV
WLNFDGEPQPYPTLPPGTGRRIHSYRGHLWLFRDAGTHDGLLVNQTELFVPSLNVDGQPIF
ANITLPVYTLKERCLQVVRSLVKPENYRRLDIVRSLYEDLEDHPNVQKDLERLTQERIAHQR
MGD
SEQ ID NO: 31
Amino acid sequence of P2-F3-VHL, an exemplary chimaeric protein of the
first aspect of the invention
MAEVQLVQSGAEVKKPGASVKVSCKASGYTFTGYYMHWVRQAPGQGLEWMGWINPNSG
GTNYAQKFQGRVTMTRDTSISTAYMELSRLRSDDTAVYYCARDRGSSWYVAFDYWGQGT
LVTVSSLEGGGGSGGGGSGGGASDIQMTQSPDSLAVSLGERATISCKSSQSVLYKYNNKN
YLAWYQQKPGQPPKLIIYWASTRESGVPDRFSGSGSGTDFTLTISSLQPEDVAVYYCQQYY
TTPYTFGQGTKVEIKRAAASAGGGGSGGGGSGGGGSGGGGSMPRRAENWDEAEVGAEE
AGVEEYGPEEDGGEESGAEESGPEESGPEELGAEEEMEAGRPRPVLRSVNSREPSQVIFC
NRSPRVVLPVWLNFDGEPQPYPTLPPGTGRRIHSYRGHLWLFRDAGTHDGLLVNQTELFV
PSLNVDGQPIFANITLPVYTLKERCLQVVRSLVKPENYRRLDIVRSLYEDLEDHPNVQKDLE
RLTQERIAHQRMGD
SEQ ID NO: 32
Amino acid sequence of P2-E2-UBOX, an exemplary chimaeric protein of the
first aspect of the invention
MAEVQLLESGGGSVQPGGSLRLSCAASGFTFSSYAMSWVRQAPGKGLEWVSAISGSGGS
TYYADSVKGRFTISRDNSKNTLYLQMNSLRAEDTAVYYCARDRDYGDYFDYWGQGTLVTV
SSLEGGGGSGGGGSGGGASDIVMTQSPGTLSLSPGERATLSCRASQTVSTYLAWYQQKP
GQAPRLLIYGASSLATGIPDRFSGSGSGTDFTLTISRLEPEDAAVYYCQQYGSSVITFGQGT
KLEIKRAAASAGGGGSGGGGSGGGGSGGGGSRLNFGDDIPSALRIAKKKRWNSIEERRIH
QESELHSYLSRLIAAERERELEECQRNHEGDEDDSHVRAQQACIEAKHDKYMADMDELFS
QVDEKRKKRDIPDYLCGKISFELMREPCITPSGITYDRKDIEEHLQRVGHFDPVTRSPLTQE
QLIPNLAMKEVIDAFISENGWVEDY
SEQ ID NO: 33
Amino acid sequence of P2-F3-UBOX, an exemplary chimaeric protein of the
first aspect of the invention
MAEVQLVQSGAEVKKPGASVKVSCKASGYTFTGYYMHWVRQAPGQGLEWMGWINPNSG
GTNYAQKFQGRVTMTRDTSISTAYMELSRLRSDDTAVYYCARDRGSSWYVAFDYWGQGT
LVTVSSLEGGGGSGGGGSGGGASDIQMTQSPDSLAVSLGERATISCKSSQSVLYKYNNKN
YLAWYQQKPGQPPKLIIYWASTRESGVPDRFSGSGSGTDFTLTISSLQPEDVAVYYCQQYY
TTPYTFGQGTKVEIKRAAASAGGGGSGGGGSGGGGSGGGGSRLNFGDDIPSALRIAKKKR
WNSIEERRIHQESELHSYLSRLIAAERERELEECQRNHEGDEDDSHVRAQQACIEAKHDKY
MADMDELFSQVDEKRKKRDIPDYLCGKISFELMREPCITPSGITYDRKDIEEHLQRVGHFDP
VTRSPLTQEQLIPNLAMKEVIDAFISENGWVEDY
SEQ ID NO: 34
Amino acid sequence of iDAb RAS-UBOX, an exemplary chimaeric protein of
the first aspect of the invention
MLCCMRRTKQVEKNDEDQKIVDMDYKDDDDRPMAEVQLLESGGGLVQPGGSLRLSCAAS
GFTFSTFSMNWVRQAPGKGLEWVSYISRTSKTIYYADSVKGRFTISRDNSKNTLYLQMNSL
RAEDTAVYYCARGRFFDYWGQGTLVTVSSLEGGGGSAAARLNFGDDIPSALRIAKKKRWN
SIEERRIHQESELHSYLSRLIAAERERELEECQRNHEGDEDDSHVRAQQACIEAKHDKYMA
DMDELFSQVDEKRKKRDIPDYLCGKISFELMREPCITPSGITYDRKDIEEHLQRVGHFDPVT
RSPLTQEQLIPNLAMKEVIDAFISENGWVEDY
SEQ ID NO: 35
Amino acid sequence of VHL-IDAb RAS, an exemplary chimaeric protein of
the first aspect of the invention
MPRRAENWDEAEVGAEEAGVEEYGPEEDGGEESGAEESGPEESGPEELGAEEEMEAGR
PRPVLRSVNSREPSQVIFCNRSPRVVLPVWLNFDGEPQPYPTLPPGTGRRIHSYRGHLWLF
RDAGTHDGLLVNQTELFVPSLNVDGQPIFANITLPVYTLKERCLQVVRSLVKPENYRRLDIV
RSLYEDLEDHPNVQKDLERLTQERIAHQRMGDLEGGGGSAAAMDYKDDDDKTSMAEVQL
LESGGGLVQPGGSLRLSCAASGFTFSTFSMNWVRQAPGKGLEWVSYISRTSKTIYYADSV
KGRFTISRDNSKNTLYLQMNSLRAEDTAVYYCARGRFFDYWGQGTLVTVSSKLNPPDESG
PGCMSCKCVLS
SEQ ID NO: 36
DNA sequence of primer DUSP6For
CTCGGATCACTGGAGCCAAAAC
SEQ ID NO: 37
DNA sequence of primer DUSP6Rev
GTCACAGTGACTGAGCGGCTAA
SEQ ID NO: 38
DNA sequence of primer GAPDHFor
GTCTCCTCTGACTTCAACAGCG
SEQ ID NO: 39
DNA sequence of primer GAPDHRev
ACCACCCTGTTGCTGTAGCCAA
SEQ ID NO: 40
Amino acid sequence of optional sequence within certain exemplary proteins
of the invention
VDGGS
SEQ ID NO: 41
Amino acid sequence of optional FLAG tag sequence in exemplary proteins
of the invention
DYKDDDDK

The following paragraphs are not claims, but are intended to assist in the understanding of the present invention, and identify subject matter for which protection may be sought in connection with the present disclosure.

1. A chimaeric protein comprising a ubiquitin ligase domain and a RAS-specific endogenous targeting portion.

2. A chimaeric protein according to paragraph 1, wherein the ubiquitin ligase domain is selected from the group consisting of: a VHL E3 ligase domain, or a fragment or variant thereof having ubiquitin ligase activity; and a UBOX domain of CHIP, or a fragment or variant thereof having ubiquitin ligase activity.

3. A chimaeric protein according to paragraph 2, comprising a VHL E3 ligase domain, or a fragment or variant thereof having ubiquitin ligase activity.

4. A chimaeric protein according to any preceding paragraph, wherein the ubiquitin ligase domain comprises the amino acid sequence set out in SEQ ID NO: 15.

5. A chimaeric protein according to any preceding paragraph, wherein the RAS-specific endogenous targeting portion is selected from the group consisting of: a RAS-specific DARPin; and a RAS-specific intracellular antibody.

6. A chimaeric protein according to any preceding paragraph, wherein the endogenous targeting portion is a KRAS-specific endogenous targeting portion selected from the group consisting of: a KRAS-specific DARPin; and a KRAS-specific intracellular antibody.

7. A chimaeric protein according to any preceding paragraph, wherein the KRAS-specific endogenous targeting portion comprises a KRAS-specific DARPin.

8. A chimaeric protein according to any preceding paragraph, wherein the KRAS-specific endogenous targeting portion comprises the amino acid sequence set out in SEQ ID NO: 6 or

8; a KRAS-binding variant of SEQ ID NO:6 or 8; or a KRAS-binding fragment of SEQ ID NO: 6 or 8 or their variants.

9. A chimaeric protein according to paragraph 8, wherein the KRAS-specific portion consists of the amino acid sequence set out in SEQ ID NO: 6 or 8; or a KRAS-binding fragment thereof.

10. A chimaeric protein according to any of paragraphs 6 to 9, wherein the KRAS-specific endogenous targeting portion is capable of binding to both mutant KRAS and wild-type KRAS.

11. A chimaeric protein according to any preceding paragraph, sharing at least 85% identity with the amino acid sequence of SEQ ID NO: 20.

12. A chimaeric protein according to paragraph 11, comprising the amino acid sequence of SEQ ID NO: 20.

13. A chimaeric protein according to paragraph 11, consisting of the amino acid sequence of SEQ ID NO: 20.

14. A chimaeric protein according to any of paragraphs 1 to 5, wherein the endogenous targeting portion is a pan RAS-specific intracellular antibody that shares at least 85% identity with the amino acid sequence of SEQ ID NO: 4.

15. A chimaeric protein according to paragraph 14, sharing at least 85% identity with the amino acid sequence of SEQ ID NO: 5.

16. A chimaeric protein according to any preceding paragraph, wherein the ubiquitin ligase domain consists of a single domain and/or the RAS-specific endogenous targeting portion consists of a single domain.

17. A nucleic acid molecule comprising a nucleic acid sequence encoding a chimaeric protein, comprising a ubiquitin ligase domain and a RAS-specific endogenous targeting portion, as defined in any of paragraphs 1 to 16.

18. A pharmaceutical composition comprising a chimaeric protein comprising a ubiquitin ligase domain and a RAS-specific endogenous targeting portion of any of paragraphs 1 to 16, and/or a nucleic acid molecule according to paragraph 17, and a pharmaceutically acceptable carrier.

19. A chimaeric protein according to any of paragraphs 1 to 16 for use as a medicament.

20. A chimaeric protein for use according to paragraph 19 in the prevention and/or treatment of a RAS-associated disorder selected from the group consisting of: a RAS-associated cancer and a RASopathy.

21. A chimaeric protein for use according to paragraph 20 in the prevention and/or treatment of a RAS-associated cancer selected from the group consisting of: RAS-associated lung cancer; RAS-associated pancreatic cancer; RAS-associated colorectal cancer; adrenocortical carcinoma; bladder urothelial carcinoma; breast invasive carcinoma; cervical squamous cell carcinoma or endocervical adenocarcinoma; cholangiocarcinoma; colon adenocarcinoma; lymphoid neoplasm diffuse large B-cell lymphoma; oesophageal carcinoma; glioblastoma multiforme; head and neck squamous cell carcinoma; kidney chromophobe; kidney renal clear cell carcinoma; kidney renal papillary cell carcinoma; acute myeloid leukaemia; brain lower grade glioma; liver hepatocellular carcinoma; lung adenocarcinoma; lung squamous cell carcinoma; ovarian serous cystadenocarcinoma; pancreatic adenocarcinoma; pheochromocytoma or paraganglioma; prostate adenocarcinoma; rectum adenocarcinoma; sarcoma; skin cutaneous melanoma; stomach adenocarcinoma; testicular germ cell tumours; thyroid carcinoma; thymoma; uterine corpus endometrial carcinoma; uterine carcinosarcoma; and uveal melanoma.

22. A chimaeric protein for use according to paragraph 20 in the prevention and/or treatment of a RASopathies selected from the group consisting of: capillary malformation-av malformation syndrome; autoimmune lymphoproliferative syndrome; cardiofaciocutaneous syndrome; hereditary gingival fibromatosis type 1; neurofibromatosis type 1; Noonan syndrome; Costello syndrome; and Legius syndrome.

23. A chimaeric protein comprising a ubiquitin ligase domain and an LMO2-specific endogenous targeting portion.

24. A chimaeric protein according to paragraph 23, sharing at least 85% identity with the amino acid sequence of SEQ ID NO: 14.

25. A chimaeric protein according to paragraph 24, comprising the amino acid sequence of SEQ ID NO: 14.

1. A chimaeric protein comprising a ubiquitin ligase domain and a RAS-specific endogenous targeting portion.

2. The chimaeric protein according to claim 1, wherein the ubiquitin ligase domain is selected from the group consisting of: a VHL E3 ligase domain, or a fragment or variant thereof having ubiquitin ligase activity; and a UBOX domain of CHIP, or a fragment or variant thereof having ubiquitin ligase activity.

3. (canceled)

4. The chimaeric protein according to claim 1, wherein the ubiquitin ligase domain shares at least 85% identity with SEQ ID NO: 15 or wherein the ubiquitin ligase domain comprises the amino acid sequence set out in SEQ ID NO: 15.

5-6. (canceled)

7. The chimaeric protein according to claim 1, wherein the RAS-specific endogenous targeting portion is selected from the group consisting of: a pan RAS-specific endogenous targeting portion; a KRAS-specific endogenous targeting portion; an NRAS-specific endogenous targeting portion; and an HRAS-specific endogenous targeting portion, wherein, if the endogenous targeting portion is a KRAS-specific endogenous targeting portion, the endogenous targeting portion is a KRAS-specific endogenous targeting portion selected from the group consisting of: a KRAS-specific DARPin; and a KRAS-specific intracellular antibody, and optionally wherein the KRAS-specific endogenous targeting portion is capable of binding to both mutant KRAS and wild-type KRAS.

8. The chimaeric protein according to claim 1, wherein the RAS-specific endogenous targeting portion is selected from the group consisting of: a RAS-specific DARPin and a RAS-specific intracellular antibody.

9-10. (canceled)

11. The chimaeric protein according to claim 7, wherein the KRAS-specific endogenous targeting portion comprises the amino acid sequence set out in SEQ ID NO: 6 or 8; a KRAS-binding variant of SEQ ID NO:6 or 8; a KRAS-binding fragment of SEQ ID NO: 6 or 8 or their variants: a KRAS-specific endogenous targeting portion sharing at least 85% identity with the amino acid sequence of SEQ ID NO: 6 or 8: or a KRAS-binding fragment thereof.

12-15. (canceled)

16. The chimaeric protein according to claim 1, wherein the endogenous targeting portion is a pan RAS-specific endogenous targeting portion selected from the group consisting of: a pan RAS-specific intracellular antibody and a pan RAS-specific DARPin.

17. The chimaeric protein according to claim 16, wherein the pan RAS-specific endogenous targeting portion is a pan RAS-specific intracellular antibody; a pan RAS-specific endogenous targeting portion sharing at least 85% identity with the amino acid sequence of SEQ ID NO: 4: or a pan RAS-specific portion comprising the amino acid sequence set out in SEQ ID NO: 4.

18-20. (canceled)

21. The chimaeric protein according to claim 1, wherein the endogenous targeting portion is an HRAS-specific endogenous targeting portion selected from the group consisting of: an HRAS-specific intracellular antibody and an HRAS-specific DARPin.

22. (canceled)

23. The chimaeric protein according to claim 1, wherein the ubiquitin ligase domain consists of a single domain, the RAS-specific endogenous targeting portion consists of a single domain, or bother.

24-25. (canceled)

26. The chimaeric protein according to claim 1, sharing at least 85% identity with the amino acid sequence of SEQ ID NO: 20 or comprising the amino acid sequence of SEQ ID NO: 20.

27-28. (canceled)

29. The chimaeric protein according to claim 16, sharing at least 85% identity with the amino acid sequence of SEQ ID NO: 5.

30. A nucleic acid molecule comprising a nucleic acid sequence encoding a chimaeric protein as defined in claim 1.

31. (canceled)

32. The nucleic acid molecule according to claim 30, wherein the nucleic acid molecule is provided in the form of a vector comprising the nucleic acid molecule, or wherein the nucleic acid molecule is provided in the form of a lentivirus vector comprising the nucleic acid molecule.

33. (canceled)

34. A pharmaceutical composition comprising a chimaeric protein comprising a ubiquitin ligase domain and a RAS-specific endogenous targeting portion and/or a nucleic acid molecule comprising a nucleic acid sequence encoding a chimaeric protein comprising a ubiquitin ligase domain and a RAS-specific endogenous targeting portion, and a pharmaceutically acceptable carrier.

35. (canceled)

36. A method of preventing or treating a RAS-associated disorder, the method comprising providing a therapeutically effective amount of a chimaeric protein according to claim 1 to a subject in need thereof, wherein the chimaeric protein is provided by administration of the protein to the subject, or wherein the chimaeric protein is provided by administration of a nucleic acid molecule comprising a nucleic acid sequence encoding a chimaeric protein comprising a ubiquitin ligase domain and a RAS-specific endogenous targeting portion—as defined in claim 1 to the subject in need thereof.

37-38. (canceled)

39. The method according to claim 36, wherein the protein or nucleic acid molecule is provided by administration of a pharmaceutical composition comprising a chimaeric protein comprising a ubiquitin ligase domain and a RAS-specific endogenous targeting portion and/or a nucleic acid molecule comprising a nucleic acid sequence encoding a chimaeric protein comprising a ubiquitin ligase domain and a RAS-specific endogenous targeting portion, and a pharmaceutically acceptable carrier to the subject.

40. The method according to claim 36, wherein the RAS-associated disorder is selected from the group consisting of: a RAS-associated cancer; RAS-associated lung cancer; RAS-associated pancreatic cancer; RAS-associated colorectal cancer; adrenocortical carcinoma; bladder urothelial carcinoma; breast invasive carcinoma; cervical squamous cell carcinoma or endocervical adenocarcinoma; cholangiocarcinoma; colon adenocarcinoma; lymphoid neoplasm diffuse large B-cell lymphoma; oesophageal carcinoma; glioblastoma multiforme; head and neck squamous cell carcinoma; kidney chromophobe; kidney renal clear cell carcinoma; kidney renal papillary cell carcinoma; acute myeloid leukaemia; brain lower grade glioma; liver hepatocellular carcinoma; lung adenocarcinoma; lung squamous cell carcinoma; ovarian serous cystadenocarcinoma; pancreatic adenocarcinoma; pheochromocytoma or paraganglioma; prostate adenocarcinoma; rectum adenocarcinoma; sarcoma; skin cutaneous melanoma; stomach adenocarcinoma; testicular germ cell tumours; thyroid carcinoma; thymoma; uterine corpus endometrial carcinoma; uterine carcinosarcoma; and uveal melanom; a RASopathy capillary malformation-av malformation syndrome; autoimmune lymphoproliferative syndrome; cardiofaciocutaneous syndrome; hereditary gingival fibromatosis type 1; neurofibromatosis type 1; Noonan syndrome; Costello syndrome; and Legius syndrome.

41-57. (canceled)

58. A chimaeric protein comprising a ubiquitin ligase domain and an LMO2-specific endogenous targeting portion.

59. The chimaeric protein according to claim 58, wherein the ubiquitin ligase domain is selected from the group consisting of: a VHL E3 ligase domain, or a fragment or variant thereof having ubiquitin ligase activity; and a UBOX domain of CHIP, or a fragment or variant thereof having ubiquitin ligase activity, wherein the LMO2-specific endogenous targeting portion is a an LMO2-specific intracellular antibody, an LMO2-specific endogenous targeting portion sharing at least 85% identity with the amino acid sequence of SEQ ID NO: 13, or an LMO2-specific endogenous targeting portion comprising the amino acid sequence set out in SEQ ID NO: 13, and wherein the pan RAS-specific endogenous portion consists of the amino acid sequence set out in SEQ ID NO: 1 or an LMO2-binding fragment thereof, or wherein the chimaeric protein shares at least 85% identity with the amino acid sequence of SEQ ID NO: 14, comprises the amino acid sequence set out in SEQ ID NO: 14, or consists of the amino acid sequence set out in SEQ ID NO: 14.

60-67. (canceled)


SEQUENCE LISTING (41 sequences)

SEQ ID NO: 1 - protein, 188 aa, Homo sapiens [sequence omitted]

SEQ ID NO: 2 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 4 - protein, 117 aa, Artificial Sequence
  Anti-pan RAS intracellular single domain antibody iDAb RAS
MAEVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSS

SEQ ID NO: 5 - protein, 335 aa, Artificial Sequence
  Chimaeric protein UBOX-iDAb RAS
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYLEGGGGSAAAMDYKDDDDKTSMAEVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSSKLNPPDESGPGCMSCKCVLS

SEQ ID NO: 6 - protein, 157 aa, Artificial Sequence
  Anti-KRAS DARPin K19
MDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 7 - DNA, 471 bp, Artificial Sequence
  DNA encoding DARPin K19
atggatctgggaaaaaaactgctggaagccgcgcgtgccgggcaggacgatgaggtccgtattcttatggcgaatggtgcagatgttaacgcgagcgatcgttggggttggacgccgctgcacctggcagcgtggtggggtcacctcgaaattgtggaagtgctgttgaagcgcggtgcagatgttagcgcggcagatctgcacggtcaatcgccgctgcatctggcagcgatggtcggccacctcgaaattgtggaagtgctgttgaagtacggtgcagatgttaacgcgaaagatacgatgggtgcaacgccgctgcacctggcagcgcgaagcggtcacctcgaaattgtggaagagctgttgaagaacggtgcagatatgaatgctcaggataagtttggcaaaaccacgtttgatatctccactgataatggcaacgaagatttagcggaaatcctgcagaaactg

SEQ ID NO: 8 - protein, 157 aa, Artificial Sequence
  Amino acid sequence of anti-KRAS DARPin K13
MDLGKKLLEAARAGQDDEVRILMANGADVNASDRWGWTPLHLAAWWGHLEIVEVLLKHGADVNAADLHGQTPLHLAAMVGHLEIVEVLLKYGADVNAKDTMGATPLHLAAQSGHLEIVEVLLKNGADVNAQDKFGKTAFDISIDNGNEDLAEILQKL

SEQ ID NO: 9 - DNA, 471 bp, Artificial Sequence
  DNA encoding K13
atggatctgggaaaaaaactgctggaagccgcgcgtgccggacaggacgatgaggtccgtattcttatggcgaacggtgcagatgttaacgcgagcgatcgctggggttggacgccgctgcatctggcagcgtggtggggtcacctcgaaattgtggaagtgctgttgaagcacggtgcagatgttaacgcggcagatctgcacggtcaaacgccgctgcatctggcagcgatggtcggtcacctcgaaattgtggaagtgctgttgaagtacggtgcagatgttaacgcgaaagatacgatgggtgcaacgccgctgcatctggcagcgcaaagcggtcacctcgaaattgtggaagtgctgttgaagaacggtgcagatgtgaatgctcaggataagtttggcaaaaccgcgtttgatatctccattgataatggcaacgaagatttagcggaaatcctgcagaaactg

SEQ ID NO: 10 - protein, 281 aa, Artificial Sequence
  anti-KRAS scFv P2-E2
MAEVQLLESGGGSVQPGGSLRLSCAASGFTFSSYAMSWVRQAPGKGLEWVSAISGSGGSTYYADSVKGRFTISRDNSKNTLYLQMNSLRAEDTAVYYCARDRDYGDYFDYWGQGTLVTVSSLEGGGGSGGGGSGGGASDIVMTQSPGTLSLSPGERATLSCRASQTVSTYLAWYQQKPGQAPRLLIYGASSLATGIPDRFSGSGSGTDFTLTISRLEPEDAAVYYCQQYGSSVITFGQGTKLEIKRAAASAHHHHHHKLDYKDHDGDYKDHDIDYKDDDDK

SEQ ID NO: 11 - protein, 289 aa, Artificial Sequence
  anti-KRAS scFv P2-F3
MAEVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSSWYVAFDYWGQGTLVTVSSLEGGGGSGGGGSGGGASDIQMTQSPDSLAVSLGERATISCKSSQSVLYKYNNKNYLAWYQQKPGQPPKLIIYWASTRESGVPDRFSGSGSGTDFTLTISSLQPEDVAVYYCQQYYTTPYTFGQGTKVEIKRAAASAHHHHHHKLDYKDHDGDYKDHDIDYKDDDDK

SEQ ID NO: 12 - protein, 158 aa, Homo sapiens
MSSAIERKSLDPSEEPVDEVLQIPPSLLTCGGCQQNIGDRYFLKAIDQYWHEDCLSCDLCGCRLGEVGRRLYYKLGRKLCRRDYLRLFGQDGLCASCDKRIRAYEMTMRVKDKVYHLECFKCAACQKHFCVGDRYLLINSDIVCEQDIYEWTKINGMI

SEQ ID NO: 13 - protein, 126 aa, Artificial Sequence
  anti-LMO2 VH576
MAEVQLLESGGGLVQPGGSLRLSCAASGFSFSHSPMNWVRQAPGKGLEWVSYISYNSSSIYYADSVKGRFTISRDNSKNTLYLQMNSLRAEDTAVYYCARGLTESLELTADWFDYWGQGTLVTVSS

SEQ ID NO: 14 - protein, 376 aa, Artificial Sequence
  Amino acid sequence of VHL-VH576, an exemplary chimaeric protein of the second aspect of the invention
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSGGGGSGGGGSAARMAEVQLLESGGGLVQPGGSLRLSCAASGFSFSHSPMNWVRQAPGKGLEWVSYISYNSSSIYYADSVKGRFTISRDNSKNTLYLQMNSLRAEDTAVYYCARGLTESLELTADWFDYWGQGTLVTVSSAAAEQKLISEEDLNGAA

SEQ ID NO: 15 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 16 - DNA, 639 bp, Homo sapiens
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgccagtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagat

SEQ ID NO: 17 - protein, 177 aa, Homo sapiens
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 18 - DNA, 531 bp, Homo sapiens
atgcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactac

SEQ ID NO: 19 - protein, 5 aa, Artificial Sequence
  Amino acid sequence of the linker sequence within VHL-DP-KRAS
GGGGS

SEQ ID NO: 20 - protein, 393 aa, Artificial Sequence
  Amino acid sequence of VHL-DP KRAS (also referred to as VHL-K19), an exemplary chimaeric protein of the first aspect of the invention
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSAAAMDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLVDGGSDYKDDDDK

SEQ ID NO: 21 - DNA, 1182 bp, Artificial Sequence
  DNA sequence encoding protein VHL-DP KRAS
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgccagtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagatctcgagggcggaggcggatctgcggccgcaatggatctgggaaaaaaactgctggaagccgcgcgtgccgggcaggacgatgaggtccgtattcttatggcgaatggtgcagatgttaacgcgagcgatcgttggggttggacgccgctgcacctggcagcgtggtggggtcacctcgaaattgtggaagtgctgttgaagcgcggtgcagatgttagcgcggcagatctgcacggtcaatcgccgctgcatctggcagcgatggtcggccacctcgaaattgtggaagtgctgttgaagtacggtgcagatgttaacgcgaaagatacgatgggtgcaacgccgctgcacctggcagcgcgaagcggtcacctcgaaattgtggaagagctgttgaagaacggtgcagatatgaatgctcaggataagtttggcaaaaccacgtttgatatctccactgataatggcaacgaagatttagcggaaatcctgcagaaactggtcgacggcgggtctgactacaaagacgacgatgacaagtaa

SEQ ID NO: 22 - protein, 393 aa, Artificial Sequence
  Amino acid sequence of VHL-K13, an exemplary chimaeric protein of the first aspect of the invention
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSAAAMDLGKKLLEAARAGQDDEVRILMANGADVNASDRWGWTPLHLAAWWGHLEIVEVLLKHGADVNAADLHGQTPLHLAAMVGHLEIVEVLLKYGADVNAKDTMGATPLHLAAQSGHLEIVEVLLKNGADVNAQDKFGKTAFDISIDNGNEDLAEILQKLVDGGSDYKDDDDK

SEQ ID NO: 23 - DNA, 1182 bp, Artificial Sequence
  DNA sequence encoding VHL-K13
atgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgccagtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagatctcgagggcggaggcggatctgcggccgcaatggatctgggaaaaaaactgctggaagccgcgcgtgccggacaggacgatgaggtccgtattcttatggcgaacggtgcagatgttaacgcgagcgatcgctggggttggacgccgctgcatctggcagcgtggtggggtcacctcgaaattgtggaagtgctgttgaagcacggtgcagatgttaacgcggcagatctgcacggtcaaacgccgctgcatctggcagcgatggtcggtcacctcgaaattgtggaagtgctgttgaagtacggtgcagatgttaacgcgaaagatacgatgggtgcaacgccgctgcatctggcagcgcaaagcggtcacctcgaaattgtggaagtgctgttgaagaacggtgcagatgtgaatgctcaggataagtttggcaaaaccgcgtttgatatctccattgataatggcaacgaagatttagcggaaatcctgcagaaactggtcgacggcgggtctgactacaaagacgacgatgacaagtaa

SEQ ID NO: 24 - protein, 357 aa, Artificial Sequence
  Amino acid sequence of UBOX-DP-KRAS, an exemplary chimaeric protein of the first aspect of the invention
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYLEGGGGSAAAMDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLVDGGSDYKDDDDK

SEQ ID NO: 25 - DNA, 1071 bp, Artificial Sequence
  DNA sequence encoding UBOX-DP-KRAS
atgcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactacctcgagggcggaggcggatctgcggccgcaatggatctgggaaaaaaactgctggaagccgcgcgtgccgggcaggacgatgaggtccgtattcttatggcgaatggtgcagatgttaacgcgagcgatcgttggggttggacgccgctgcacctggcagcgtggtggggtcacctcgaaattgtggaagtgctgttgaagcgcggtgcagatgttagcgcggcagatctgcacggtcaatcgccgctgcatctggcagcgatggtcggccacctcgaaattgtggaagtgctgttgaagtacggtgcagatgttaacgcgaaagatacgatgggtgcaacgccgctgcacctggcagcgcgaagcggtcacctcgaaattgtggaagagctgttgaagaacggtgcagatatgaatgctcaggataagtttggcaaaaccacgtttgatatctccactgataatggcaacgaagatttagcggaaatcctgcagaaactggtcgacggcgggtctgactacaaagacgacgatgacaag

SEQ ID NO: 26 - protein, 484 aa, Artificial Sequence
  Amino acid sequence of VHL-P2-E2, an exemplary chimaeric protein of the first aspect of the invention
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGSGGGGSGGGGSGGGGSMAEVQLLESGGGSVQPGGSLRLSCAASGFTFSSYAMSWVRQAPGKGLEWVSAISGSGGSTYYADSVKGRFTISRDNSKNTLYLQMNSLRAEDTAVYYCARDRDYGDYFDYWGQGTLVTVSSLEGGGGSGGGGSGGGASDIVMTQSPGTLSLSPGERATLSCRASQTVSTYLAWYQQKPGQAPRLLIYGASSLATGIPDRFSGSGSGTDFTLTISRLEPEDAAVYYCQQYGSSVITFGQGTKLEIKRAAASA

SEQ ID NO: 27 - protein, 492 aa, Artificial Sequence
  Amino acid sequence of VHL-P2-F3, an exemplary chimaeric protein of the first aspect of the invention
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGSGGGGSGGGGSGGGGSMAEVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSSWYVAFDYWGQGTLVTVSSLEGGGGSGGGGSGGGASDIQMTQSPDSLAVSLGERATISCKSSQSVLYKYNNKNYLAWYQQKPGQPPKLIIYWASTRESGVPDRFSGSGSGTDFTLTISSLQPEDVAVYYCQQYYTTPYTFGQGTKVEIKRAAASA

SEQ ID NO: 28 - protein, 448 aa, Artificial Sequence
  Amino acid sequence of UBOX-P2-E2, an exemplary chimaeric protein of the first aspect of the invention
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGGGGSGGGGSGGGGSGGGGSMAEVQLLESGGGSVQPGGSLRLSCAASGFTFSSYAMSWVRQAPGKGLEWVSAISGSGGSTYYADSVKGRFTISRDNSKNTLYLQMNSLRAEDTAVYYCARDRDYGDYFDYWGQGTLVTVSSLEGGGGSGGGGSGGGASDIVMTQSPGTLSLSPGERATLSCRASQTVSTYLAWYQQKPGQAPRLLIYGASSLATGIPDRFSGSGSGTDFTLTISRLEPEDAAVYYCQQYGSSVITFGQGTKLEIKRAAASA

SEQ ID NO: 29 - protein, 456 aa, Artificial Sequence
  Amino acid sequence of UBOX-P2-F3, an exemplary chimaeric protein of the first aspect of the invention
MRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYGGGGSGGGGSGGGGSGGGGSMAEVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSSWYVAFDYWGQGTLVTVSSLEGGGGSGGGGSGGGASDIQMTQSPDSLAVSLGERATISCKSSQSVLYKYNNKNYLAWYQQKPGQPPKLIIYWASTRESGVPDRFSGSGSGTDFTLTISSLQPEDVAVYYCQQYYTTPYTFGQGTKVEIKRAAASA

SEQ ID NO: 30 - protein, 484 aa, Artificial Sequence
  Amino acid sequence of P2-E2-VHL, an exemplary chimaeric protein of the first aspect of the invention
MAEVQLLESGGGSVQPGGSLRLSCAASGFTFSSYAMSWVRQAPGKGLEWVSAISGSGGSTYYADSVKGRFTISRDNSKNTLYLQMNSLRAEDTAVYYCARDRDYGDYFDYWGQGTLVTVSSLEGGGGSGGGGSGGGASDIVMTQSPGTLSLSPGERATLSCRASQTVSTYLAWYQQKPGQAPRLLIYGASSLATGIPDRFSGSGSGTDFTLTISRLEPEDAAVYYCQQYGSSVITFGQGTKLEIKRAAASAGGGGSGGGGSGGGGSGGGGSMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 31 - protein, 492 aa, Artificial Sequence
  Amino acid sequence of P2-F3-VHL, an exemplary chimaeric protein of the first aspect of the invention
MAEVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSSWYVAFDYWGQGTLVTVSSLEGGGGSGGGGSGGGASDIQMTQSPDSLAVSLGERATISCKSSQSVLYKYNNKNYLAWYQQKPGQPPKLIIYWASTRESGVPDRFSGSGSGTDFTLTISSLQPEDVAVYYCQQYYTTPYTFGQGTKVEIKRAAASAGGGGSGGGGSGGGGSGGGGSMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 32 - protein, 447 aa, Artificial Sequence
  Amino acid sequence of P2-E2-UBOX, an exemplary chimaeric protein of the first aspect of the invention
MAEVQLLESGGGSVQPGGSLRLSCAASGFTFSSYAMSWVRQAPGKGLEWVSAISGSGGSTYYADSVKGRFTISRDNSKNTLYLQMNSLRAEDTAVYYCARDRDYGDYFDYWGQGTLVTVSSLEGGGGSGGGGSGGGASDIVMTQSPGTLSLSPGERATLSCRASQTVSTYLAWYQQKPGQAPRLLIYGASSLATGIPDRFSGSGSGTDFTLTISRLEPEDAAVYYCQQYGSSVITFGQGTKLEIKRAAASAGGGGSGGGGSGGGGSGGGGSRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 33 - protein, 455 aa, Artificial Sequence
  Amino acid sequence of P2-F3-UBOX, an exemplary chimaeric protein of the first aspect of the invention
MAEVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDRGSSWYVAFDYWGQGTLVTVSSLEGGGGSGGGGSGGGASDIQMTQSPDSLAVSLGERATISCKSSQSVLYKYNNKNYLAWYQQKPGQPPKLIIYWASTRESGVPDRFSGSGSGTDFTLTISSLQPEDVAVYYCQQYYTTPYTFGQGTKVEIKRAAASAGGGGSGGGGSGGGGSGGGGSRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 34 - protein, 335 aa, Artificial Sequence
  Amino acid sequence of iDAb RAS-UBOX, an exemplary chimaeric protein of the first aspect of the invention
MLCCMRRTKQVEKNDEDQKIVDMDYKDDDDRPMAEVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSSLEGGGGSAAARLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 35 - protein, 371 aa, Artificial Sequence
  Amino acid sequence of VHL-iDAb RAS, an exemplary chimaeric protein of the first aspect of the invention
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDLEGGGGSAAAMDYKDDDDKTSMAEVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSSKLNPPDESGPGCMSCKCVLS

SEQ ID NO: 36 - DNA, 22 bp, Artificial Sequence
  DNA sequence of primer DUSP6For
ctcggatcactggagccaaaac

SEQ ID NO: 37 - DNA, 22 bp, Artificial Sequence
  DNA sequence of primer DUSP6Rev
gtcacagtgactgagcggctaa

SEQ ID NO: 38 - DNA, 22 bp, Artificial Sequence
  DNA sequence of primer GAPDHFor
gtctcctctgacttcaacagcg

SEQ ID NO: 39 - DNA, 22 bp, Artificial Sequence
  DNA sequence of primer GAPDHRev
accaccctgttgctgtagccaa

SEQ ID NO: 40 - protein, 5 aa, Artificial Sequence
  Amino acid sequence of optional sequence within certain exemplary proteins of the invention
VDGGS

SEQ ID NO: 41 - protein, 8 aa, Artificial Sequence
  Amino acid sequence of optional FLAG tag sequence in exemplary proteins of the invention
DYKDDDDK